(19) United States
(12) Patent Application Publication
(10) Pub. No.: US 2023/0323487 A1
(43) Pub. Date: Oct. 12, 2023

(54) PORTABLE DETECTION OF SARS-COV-2 USING UNIMOLECULAR APTASENSORS

(71) Applicant: ARIZONA BOARD OF REGENTS ON BEHALF OF ARIZONA STATE UNIVERSITY, Scottsdale, AZ (US)

(72) Inventors: Alexander GREEN, Chestnut Hill, MA (US); Zhaoqing YAN, Tempe, AZ (US); Anli TANG, Gilbert, AZ (US)

(21) Appl. No.: 18/043,012

(22) PCT Filed: Aug. 26, 2021
(86) PCT No.: PCT/US2021/047675

§ 371 Date: Feb. 24, 2023

Related U.S. Application Data
(60) Provisional application No. 63/070,543, filed on Aug. 26, 2020.

(51) Int. Cl. C12Q 1/70; C12Q 1/6825; C12N 15/115; C12Q 1/6818

(52) U.S. Cl. CPC C12Q 1/701; C12Q 1/6825; C12N 2310/16; C12Q 1/6818; C12N 15/115

(57) ABSTRACT

Provided herein are methods and compositions for rapid, highly sensitive detection of SARS-CoV-2, the causative agent of the COVID-19 pandemic or other target nucleic acids. The methods are low-cost and can be implemented in a portable format that does not require elaborate biosafety precautions or sophisticated laboratory equipment.

### CROSS-REFERENCE TO RELATED APPLICATIONS

This application claims priority to U.S. Provisional Patent Application No. 63/070,543, filed Aug. 26, 2020, the contents of which are incorporated by reference in their entireties.

### STATEMENT REGARDING FEDERALLY FUNDED RESEARCH OR DEVELOPMENT

This invention was made with government support under GM126892 and R21 AI136571 awarded by the National Institutes of Health and 2029532 awarded by the National Science Foundation. The government has certain rights in the invention.

### SEQUENCE LISTING

A Sequence Listing accompanies this application.

### BACKGROUND

Low-cost and easy-to-use tests to detect SARS-CoV-2, the causative agent of the COVID-19 pandemic, are essential tools to contain the spread of the virus and ensure that patients receive timely treatment. Such tests can be implemented at the point of care or in the home to enable distributed testing and more rapid results. Conventional PCR-based testing, however, is limited to centralized labs with sophisticated equipment. The increasing demand for PCR-based testing reagents further suggests that diagnostic assays that utilize reagents outside the PCR pipeline could be valuable tools to increase testing capacity.

Survey of conventional diagnostics currently approved for use in the United States reveals that they require multiple days to return results, they require expensive equipment, or they lack sensitivity and specificity. Moreover, tests require trained personnel to run them, making in-home use challenging. These requirements substantially increase both the cost and time required to return assay results. Accordingly, there remains a need in the art for rapid, inexpensive, and highly sensitive diagnostic tests for SARS-CoV-2, the causative agent of the COVID-19 pandemic, that require neither sophisticated laboratory equipment nor biosafety level 3 containment.

### SUMMARY

In a first aspect, the present invention provides aptasensors for detecting SARS-CoV-2. The aptasensors comprise: (a) a target-binding sequence that is complementary to a SARS-CoV-2 target nucleic acid or to the complement thereof; and (b) an aptamer. In the absence of the SARS-CoV-2 target nucleic acid, the aptasensor forms a stem-loop structure in which a first portion of the target-binding sequence forms a single-stranded toehold and a second portion of the target-binding sequence base-pairs with a portion of the aptamer to form a stem, such that the aptamer cannot fold into its active form. However, binding of the target-binding sequence to the SARS-CoV-2 target nucleic acid disrupts the stem-loop structure, allowing the aptamer to fold into its active form and bind to its cognate ligand.

In a second aspect, the present invention provides methods of detecting SARS-CoV-2 in a sample. The methods comprise: (a) amplifying the SARS-CoV-2 target nucleic acid in the sample; (b) contacting the amplified nucleic acid with an aptasensor disclosed herein and the cognate ligand of its aptamer; and (c) detecting any signal produced by the aptamer binding to its cognate ligand. In these methods, detection of the signal indicates that SARS-CoV-2 is present in the sample.

In a third aspect, the present invention provides kits for detecting SARS-CoV-2 comprising the aptasensors disclosed herein.

### BRIEF DESCRIPTION OF THE DRAWINGS

The patent or patent application file contains at least one drawing in color. Copies of this patent or patent application publication with color drawings will be provided by the Office upon request and payment of the necessary fee.

FIG. 1 is a schematic illustration of a method for detecting SARS-CoV-2. In this method, viral RNA is extracted from patient samples and is amplified using an isothermal amplification method. The amplified nucleic acids are then detected using a SARS-CoV-2-specific aptasensor, which produces a strong fluorescence signal if SARS-CoV-2 RNA is present in the patient sample.

FIG. 2 shows schematic illustrations of aptasensors used for SARS-CoV-2 detection. (A) The aptasensors disclosed herein are designed to target the SARS-CoV-2 genes Orflb, RdRP, E, and N. (B) In aptasensors based on the Broccoli aptamer, a 5′ hairpin sequence is used to prevent formation of the Broccoli aptamer structure at the 3′ end of the RNA. A toehold-mediated interaction is used to initiate binding with the SARS-CoV-2 target RNA to unwind the hairpin stem. Unwinding of the hairpin stem enables formation of the Broccoli aptamer. The aptamer then binds to the fluorogen DFHBI-1T, which emits green fluorescence. One to three mismatches may be present in the stem of the Broccoli aptasensor depending on the particular sensor. (C) In aptasensors based on the Corn aptamer, a 5′ hairpin sequence prevents formation of the Corn aptamer at the 3′ end of the transcript. Toehold-mediated binding to the target SARS-CoV-2 induces downstream formation of the Corn aptamer. The assembled aptamer structure binds to the fluorogen DFHO, which emits yellow fluorescence. The circled C and G in the Corn aptasensor represent conserved bases that are necessary for strong aptamer fluorescence. “*” indicates that a domain is a reverse complement.

FIG. 3 shows validation data from Broccoli aptasensors targeting the sense orientation of the RdRP gene of SARS-CoV-2. (A) ON/OFF ratios of eight different aptasensors determined in the presence or absence of the cognate target RNA after 1 or 2 hour reactions. (B) Photograph of fluorescence from the sensors measured in triplicate in the presence or absence of the cognate target RNA. (C,D) Time-course measurements of fluorescence from two high-performance aptasensors with and without the SARS-CoV-2 target RNA.

FIG. 4 shows validation data from Broccoli aptasensors targeting the antisense orientation of the E gene of SARS-CoV-2. (A) ON/OFF ratios of eight different aptasensors determined in the presence or absence of the cognate target RNA after 1 or 2 hour reactions. (B) Photograph of fluorescence from the sensors measured in triplicate in the presence or absence of the cognate target RNA. (C,D) Time-course measurements of fluorescence from two high-performance aptasensors with and without the SARS-CoV-2 target RNA.

FIG. 5 shows the ON/OFF ratios after 1 and 2 hour reactions of Broccoli aptasensors targeting different SARS-CoV-2 regions: (A) sense orientation of Orflb, (B) sense orientation of the N gene, (C) sense orientation of the E gene, and (D) sense orientation of the N gene.

FIG. 6 shows ON/OFF ratios after 1 and 2 hours reactions of Broccoli aptasensors targeting different SARS-CoV-2 regions: (A) sense orientation of the N gene, (B) antisense orientation of the Orflb, (C) antisense orientation of the N gene, and (D) antisense orientation of the RdRP gene.

FIG. 7 shows validation data from Corn aptasensors targeting the sense orientation of the Orflb region of SARS-CoV-2. (A) ON/OFF ratios of six different aptasensors determined in the presence or absence of the cognate target RNA after 1 or 2 hour reactions. (B) Photograph of fluorescence from the sensors measured in triplicate in the presence or absence of the cognate target RNA. (C,D) Time-course measurements of fluorescence from two high-performance aptasensors with and without the SARS-CoV-2 target RNA.

FIG. 8 shows validation data from Corn aptasensors targeting the sense orientation of the RdRP gene of SARS-CoV-2. (A) ON/OFF ratios of six different aptasensors determined in the presence or absence of the cognate target RNA after 1 or 2 hour reactions. (B) Photograph of fluorescence from the sensors measured in triplicate in the presence or absence of the cognate target RNA. (C, D) Time-course measurements of fluorescence from two high-performance aptasensors with and without the SARS-CoV-2 target RNA.

FIG. 9 shows ON/OFF ratios after 1 and 2 hour reactions of Corn aptasensors targeting different SARS-CoV-2 regions: (A) sense orientation of the E gene, (B) sense orientation of the N gene, and (C) sense orientation of the N gene in a different subregion.

FIG. 10 shows data from experiments in which a Broccoli aptasensor targeting the Orflb region of SARS-CoV-2 was used to detect amplicons generated using NASBA isothermal amplification of RNA obtained from cultured virions. (A) Screening of 10 different NASBA primer pairs from reactions with 185 copies of SARS-CoV-2 genomic RNA. Primer performance was assessed based on strength of aptasensor response. (B) Aptasensor response following NASBA with different template concentrations. A detection limit of 28 copies in the amplification reaction was obtained. (C) Photograph of fluorescence from aptasensor reactions following NASBA of different SARS-CoV-2 RNA template concentrations. Reactions were measured in triplicate. (D) Aptasensor response at the measured assay detection limit of 3.8 copies/μL of SARS-CoV-2 RNA (6.28 aM) in the amplification reaction.

FIG. 11 shows data from experiments in which a Broccoli aptasensor targeting the RdRP gene of SARS-CoV-2 was used to detect amplicons generated using NASBA isothermal amplification of RNA obtained from cultured virions. (A) Screening of 10 different NASBA primer pairs from reactions with 185 copies of SARS-CoV-2 genomic RNA. Primer performance was assessed based on strength of aptasensor response. (B) Aptasensor response following NASBA with different template concentrations. A detection limit of 28 copies in the amplification reaction was obtained. (C) Photograph of fluorescence from the aptasensor reactions following NASBA of different SARS-CoV-2 RNA template concentrations. Reactions were performed in triplicate.

FIG. 12 shows performance data from a library of eight Broccoli aptasensors designed to target an amplicon produced from an RT-RPA reaction amplifying the N gene of SARS-CoV-2. (A) ON/OFF ratios of the aptasensor library with and without the target RNA. (B-D) Time-course measurements obtained from a plate reader of the top-performing aptasensors with and without the target RNA at 37° C. The RNA target produced from the RT-RPA amplicon contains the antisense sequence of a region of the SARS-CoV-2 N gene.

FIG. 13 shows validation data from Broccoli aptasensors targeting the antisense orientation of the human RNase P mRNA, which can serve as a positive control for proper sample handling in viral assays. (A) ON/OFF ratios of the best aptasensor determined in the presence or absence of the cognate target RNA after 1 or 2 hour reactions. (B) Photograph of fluorescence from the sensors measured in triplicate in the presence or absence of the cognate target RNA. (C) Time-course measurements of fluorescence from the aptasensor with and without the SARS-CoV-2 target RNA.

FIG. 14 shows validation data from Corn aptasensors targeting the antisense orientation of the human RNase P mRNA, which can serve as a positive control for proper sample handling in viral assays. (A) ON/OFF ratios of the best aptasensor determined in the presence or absence of the cognate target RNA after 1 or 2 hour reactions. (B) Photograph of fluorescence from the sensors measured in triplicate in the presence or absence of the cognate target RNA. (C) Time-course measurements of fluorescence from the aptasensor with and without the SARS-CoV-2 target RNA.

FIG. 15 shows ON/OFF fluorescence data from Broccoli aptasensors targeting the loop region of DNA molecules representative of the expected products of RT-LAMP reactions amplifying a SARS-CoV-2 target nucleic acid.

FIG. 16 shows ON/OFF fluorescence data from Broccoli aptasensors targeting the loop region of DNA molecules representative of the expected products of RT-LAMP reactions amplifying the control nucleic acids human actin beta mRNA and 18S rRNA.

FIG. 17 shows ON/OFF fluorescence data from Corn aptasensors targeting the loop region of DNA molecules representative of the expected products of RT-LAMP reactions amplifying the control nucleic acids human actin beta mRNA and 18S rRNA.

FIG. 18 illustrates simple, inexpensive, and widely available components suitable for readout of aptasensor reactions by eye or using a smartphone camera. (A) A flashlight, blue light filter, and orange filter can be configured for detection of fluorescence from the Broccoli aptasensors. (B) Photograph of active Broccoli aptamers displaying green fluorescence emission with the components shown in panel A. (C) A blue light flashlight and an orange filter can be configured for detection of fluorescence from the Broccoli aptasensors. (D) Photograph of active Broccoli aptamers displaying green fluorescence with the components shown in panel C.

FIG. 19 shows a schematic of the improved aptasensor design. The aptasensor has a sensing hairpin with a stem ranging from 12 to 21 base pairs (bp) in length that does not have any bulges. The c domain is usually 6 bp long and the b domain is 6 to 18 bp long. The “inner clamp” forms a strong stem-loop specifically designed for each aptasensor that encourages formation of the active aptamer. The “outer clamp” is a stem loop structure that can form when the aptamer is in its active form to stabilize the aptamer structure via formation of a stem between the b* sequence and a complementary b′ sequence positioned 3′ to the aptamer sequence.

FIG. 20 shows representative ON/OFF data from the improved aptasensor design for a series of Red Broccoli aptasensors using the dye OBI targeting a DNA target from the SARS-CoV-2 N gene.

FIG. 21 shows representative ON/OFF data from the improved aptasensor design for a series of Orange Broccoli aptasensors targeting a DNA target from the SARS-CoV-2 N gene.

FIG. 22 shows representative ON/OFF data from the improved aptasensor design for a series of Corn aptasensors targeting a DNA target from the SARS-CoV-2 N gene and the 18s rRNA sample control gene.

FIG. 23 shows the aptasensor system designed for detection of RT-LAMP or LAMP DNA products. The aptasensor binds to exposed loop domains in the RT-LAMP or LAMP DNA amplicons to produce the output fluorescence signal.

FIG. 24 shows the scheme for parallel detection of two different RT-LAMP DNA loop domains in the same reaction. One aptasensor targets the a loop domain while the second aptasensor targets the b loop domain, enabling increases in signal and/or reaction speed.

FIG. 25 shows the fluorescence output from different combinations of aptasensors targeting either the F-loop, the B-loop, or the F-loop and B-loop of an RT-LAMP amplicon from SARS-CoV-2.

FIG. 26 shows the fluorescence signal from Broccoli aptasensors targeting SARS-CoV-2 after amplification with RT-LAMP.

FIG. 27 shows the fluorescence signal from a pair of Broccoli aptasensors targeting the ACTB sample control mRNA after amplification with RT-LAMP. The ACTB mRNA was supplied to the RT-LAMP reaction at a concentration of 0.2 aM.

FIG. 28 shows the fluorescence signal from Red Broccoli aptasensors targeting SARS-CoV-2 after amplification with RT-LAMP. The reaction volume was 30 μL.

FIG. 29 shows the fluorescence signal from Red Broccoli aptasensors targeting the ACTB sample control mRNA after amplification with RT-LAMP. The reaction volume was 30 μL.

FIG. 30 shows the photographs of the fluorescence emission from Orange Broccoli aptasensors targeting SARS-CoV-2 (left) and the ACTB sample control mRNA (right) after amplification with RT-LAMP. The reaction volume was 30 μL.

FIG. 31 shows the fluorescence signal from Corn aptasensors targeting the ACTB control mRNA after amplification with RT-LAMP. The reaction volume was 30 μL. The Human ACTB mRNA was supplied to the RT-LAMP reaction at a concentration of 0.2 aM.

FIG. 32 shows the approach for a two-channel RT-LAMP/aptasensor assay where one aptasensor targets a viral RNA and the other targets a control mRNA (ACTB) in the same reaction.

FIG. 33 shows the green (Broccoli channel) and orange (Corn channel) aptamer fluorescence signals in reactions containing RT-LAMP products. Independent fluorescence signals from Broccoli/DFHBI-1T and Corn/DFHO enable simultaneous detection of SARS-CoV-2 and ACTB RNA.

FIG. 34 shows a schematic of the multiplexable one-pot RT-LAMP/aptasensor assay.

FIG. 35 shows a one-pot RT-LAMP/aptasensor assay for detection of the SARS-CoV-2 N gene down to 10 copies in a 20-μL reaction volume.

FIG. 36 shows fluorescence data from a two-channel, one-pot RT-LAMP/aptasensor assay that enables simultaneous detection of SARS-CoV-2 RNA and an ACTB mRNA control.

FIG. 37 shows a validation of the aptasensor assay against clinical saliva samples. The assay identified 29 out of 30 positive samples correctly and 30 out of 30 negative samples correctly, which corresponds to a sensitivity of 96.67%, a specificity of 100%, and an accuracy of 98.33%.

FIG. 38 shows a validation of aptasensor assay using a rapid 98° C. RNA extraction from clinical saliva samples. The simplified assay identified 9 out 10 positive samples correctly and 9 out of 10 negative samples correctly.

FIG. 39 shows the results of a high-throughput SARS-CoV-2 assay in 384-well plates. The aptasensor assay was prepared in 384-well plates using stable master mix formulations. The rapid response of the Broccoli aptasensors against a variety of SARS-CoV-2 variants enabled positive calls to be made within 25 minutes of incubation in a plate reader. 176 out of 176 positive samples and 192 out of 192 negative samples were correctly identified. 16 wells (right-most column of plate) were used for controls. The assays did not activate in the presence of other human coronaviruses, MERS, and influenza A.

### DETAILED DESCRIPTION

The present invention provides compositions and methods for rapid, highly sensitive detection of SARS-CoV-2, the causative agent of the COVID-19 pandemic. In the methods, a SARS-CoV-2 target nucleic acid is amplified and is then bound by a sequence-specific aptasensor for detection. These methods offer several advantages. For example, the use of an aptasensor for detection confirms that the amplified nucleic acid comprises the target sequence, reducing the risk of false positive results. The aptasensors described herein produce a strong fluorescence signal that can be detected by eye or using inexpensive and readily available equipment, such as a smartphone camera. Consequently, the methods of the present invention do not need to be performed at a centralized lab. Further, the inventors have demonstrated that these methods can detect SARS-CoV-2 in samples containing as few as 2 copies of viral RNA. Aptasensors

In a first aspect, the present invention provides aptasensors for detecting SARS-CoV-2. The aptasensors comprise: (a) a target-binding sequence that is complementary to a SARS-CoV-2 target nucleic acid or to the complement thereof; and (b) an aptamer. In the absence of the SARS-CoV-2 target nucleic acid, the aptasensor forms a stem-loop structure in which a first portion of the target-binding sequence forms a single-stranded toehold and a second portion of the target-binding sequence base-pairs with a portion of the aptamer to form a stem, such that the aptamer cannot fold into its active form. However, binding of the target-binding sequence to the SARS-CoV-2 target nucleic acid disrupts the stem-loop structure, allowing the aptamer to fold into its active form and bind to its cognate ligand.

As used herein, the term “aptasensor” refers to a single-stranded oligonucleotide that functions as a molecular sensor. The aptasensors of the present invention form an inhibitory stem-loop structure that is disrupted when the aptasensor binds to a target nucleic acid, allowing the aptasensor to produce a detectable signal. The aptasensors used with the present invention may comprise single-stranded RNA or single-stranded DNA.

The aptasensors of the present invention comprise an inhibitory stem-loop. Within this stem-loop, the stem is typically about 10-25 nucleotides in length. In some embodiments, the stem is 12-21 nucleotides in length. In some embodiments, the stem is about 20 nucleotides in length. In some embodiments, the stem comprises bulges, i.e., non-base paired nucleotides within the stem. For example, the aptasensors described in Example 1 comprise a stem with two bulges that are four and eight bases from the top base pair of the stem. In other embodiments (exemplified by the aptasensors described in Example 2), the stem does not comprise bulges. The loop of the inhibitory stem-loop structure may be about 6-10 nucleotides in length and is typically about 8 nucleotides in length. However, the length of the loop may be decreased to make the hairpin stronger or be increased to make the hairpin weaker due to entropic effects.

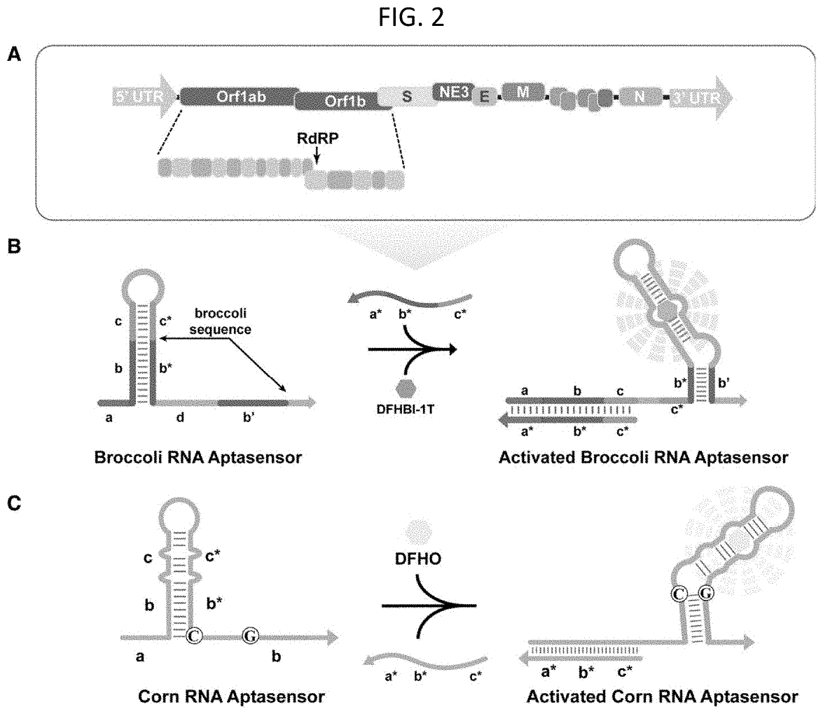

The aptasensors of the present invention comprise two functional components: a target-binding sequence and an aptamer. The “target-binding sequence” is an oligonucleotide that is complementary to a SARS-CoV-2 target nucleic acid or to the complement thereof. Within the unactivated aptasensor structure, a first portion of the target-binding sequence exists as a toehold (i.e., a single-stranded overhang), while a second portion forms a stem by base-pairing with a complementary portion of the aptamer. Binding of the toehold to a target nucleic acid thermodynamically drives the remaining stem-forming portion of the target-binding sequence to bind to the target nucleic acid, disrupting the stem-loop structure of the aptasensor. The toehold portion of the target-binding sequence should be at least 4 nucleotides in length. In some embodiments, the toehold is 8-30 nucleotides in length. In certain embodiments, the toehold is 15 nucleotides in length. The portion of the target-binding sequence that forms a stem by base-pairing with a complementary portion of the aptamer (i.e., the b domain in FIG. 2B) is typically 6-12 base nucleotides in length.

The “aptamer” portion of the aptasensor is an oligonucleotide that is capable of binding to a specific cognate ligand when it is in its active form. An aptamer is in its “active form” when it has folded into the proper three-dimensional structure for binding to its cognate ligand.

As is schematically depicted in FIG. 2, binding of an aptasensor to the SARS-CoV-2 target nucleic acid initiates a conformational change in the aptasensor that results in the generation of a detectable signal. In the absence of the target nucleic acid, the aptasensors form a stem-loop structure in which a portion of the aptamer is base-paired with a complementary sequence in the target-binding sequence. This structure sequesters that portion of the aptamer, preventing the aptamer from assuming its active form. However, binding of the target-binding sequence to the SARS-CoV-2 target nucleic acid disrupts this base-pairing, freeing the aptamer from the inhibitory stem-loop. The aptamer then folds into its active form, such that it is available to bind to its cognate ligand.

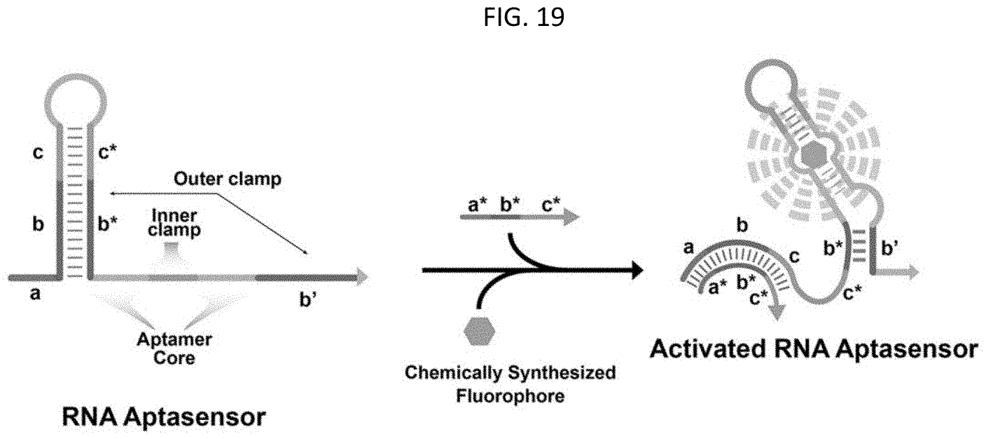

In some embodiments, the aptamer comprises an inner clamp within the aptamer core, as depicted in FIG. 19. As used herein, the term “aptamer core” refers to a middle portion of the aptamer that lies between a first portion of the aptamer that forms part of the stem in the stem-loop structure (labeled as “b*” in FIG. 19) and the portion of the aptamer that base pairs with this first portion (labeled as “b’” in FIG. 19) to form the active aptamer structure. The “inner clamp” is a sequence that forms a strong stem-loop to encourage formation of the active aptamer structure. The stem of the inner clamp stem-loop may be about 6-14 nucleotides in length and the loop of the inner clamp stem-loop structure may be about 4-10 nucleotides in length. In particular embodiments, the stem is 8 nucleotides in length and the loop is 4 nucleotides in length. As shown in FIG. 19 these aptamers may also contain an “outer clamp”. The outer clamp helps stabilize the aptamer in its active form by forming a stem at the base of the aptamer between the b* section of the aptamer and a complementary b′ section as shown in FIG. 19. This stem is generally the same length as the b domain. The b′ sequence is located 3′ to the aptamer sequence such that the stem formed by b*-b′ when the aptamer is in its active form is at the base of the aptamer.

The aptamers of the present invention serve as reporters in that they produce a detectable signal upon binding to their cognate ligand. A “detectable signal” is a signal that can be detected over any background noise. Suitable detectable signals include, without limitation, fluorescence signals, luminescence signals, colorimetric signals, wavelength absorbance, and radioactive signals.

In some embodiments, the detectable signal is a colorimetric signal. A “colorimetric signal” is a signal that produces a color change. One example of a system that generates a colorimetric signal is 2,2′-azino-bis(3-ethylbenzothiazoline-6-sulfonic acid) diammonium salt (ABTS) system, wherein ABTS interacts with a DNA catalyst to generate a colored byproduct. Advantageously, a colorimetric signal may be visible by eye, such that no special equipment is required to visualize it. However, in some cases, it may be desirable to quantify the colorimetric signal using a device such as a spectrophotometer.

In some embodiments, the detectable signal is a fluorescence signal. “Fluorescence” is the emission of light by a substance that has absorbed light or another form of electromagnetic radiation. Any aptamer that produces a fluorescence signal upon binding to its cognate ligand may be used with the present invention. In Example 1, the inventors utilize the aptamers Broccoli and Corn in their aptasensors. Thus, in some embodiments, the aptamer is Broccoli or Corn. The binding of these aptamers activates the fluorescence of their cognate ligands (see FIG. 2). Broccoli is a 49-nucleotide RNA aptamer that binds to the cognate ligand 3,5-difluoro-4-hydroxybenzylidene imidazolinone (DFHBI), which is a fluorophore derived from GFP, or to a derivative thereof (e.g., DFHBI-1T). See Song et al., J. Am. Chem. Soc. 2014, 136:1198. Corn is an RNA aptamer that binds to the cognate ligand 3,5-difluoro-4-hydroxybenzylidene-imidazolinone-2-oxime (DFHO), which is a fluorophore derived from DsRed. See Song et al., Nat Chem Biol. 2017, 13(11): 1187-1194. In Example 2, the inventors further utilize the aptamers Red Broccoli and Orange Broccoli, which are derivatives of the Broccoli aptamer that produce red-shifted fluorescence (Song et al., Nature Chemical Biology 2017, 13, 1187). Thus, in some embodiments, the aptamer is Red Broccoli or Orange Broccoli. Other suitable aptamers that produce a fluorescence signal include, without limitation, Spinach and Spinach2 (Strack et al., Nature Methods 2013, 10:1219-1224), Carrot and Radish (Paige et al., Science 2011, 333:642-646), RT aptamer (Sato et al., Angew. Chem. Int. Ed. 2014, 54:1855-1858), hemin-binding G-quadruplex DNA and RNA aptamers, and malachite green binding aptamer (Babendure et al., J. Am. Chem. Soc. 2003).

The SARS-CoV-2 target nucleic acid that is bound by the aptasensors may comprise any portion of the SARS-CoV-2 genome. The SARS-CoV-2 genome is comprised of single-stranded positive-sense RNA. Suitable target sequences include those found in any of the major genes (i.e., the S, E, M, and N genes), in any of the 13-15 open reading frames, or in any non-coding region of the SARS-CoV-2 genome. Ideally, the target nucleic acid comprises a sequence that is specific to SARS-CoV-2, meaning that it is not present in the genome of other organisms. In the Examples, the inventors designed aptasensors that detect the SARS-CoV-2 genes Orflb, RdRp, spike, E, and N. The sequences of their aptasensors are provided in Tables 1-7, 10, 12, and 15-18 as SEQ ID NOs:1-118, 121-136, and 151-248. Thus, in some embodiments, the SARS-CoV-2 target nucleic acid is a portion of a SARS-CoV-2 gene selected from the group consisting of: Orflb, RdRp, spike, E, and N. In some embodiments, the aptasensor comprises a sequence selected from SEQ ID NOs:1-118, 121-136, and 151-248. However, the aptasensors provided herein (i.e., SEQ ID NOs:1-118, 121-136, and 151-248) can tolerate mutations, particularly in the toehold domain. The aptasensors may also comprise mutations in the b and c stem-forming domains (see FIG. 2B), which need not be fully complementary to the target nucleic acid. Thus, in other embodiments, the aptasensor comprises a sequence with at least 80%, at least 85%, at least 90%, at least 95%, at least 97%, at least 98%, or at least 99% identity to a sequence selected from SEQ ID NOs: 1-118, 121-136, and 151-248.

“Percentage of sequence identity” is determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity. Protein and nucleic acid sequence identities are evaluated using the Basic Local Alignment Search Tool (“BLAST”), which is well known in the art (Karlin and Altschul, 1990, Proc. Natl. Acad. Sci. USA 87: 2267-2268; Altschul et al., 1997, Nucl. Acids Res. 25: 3389-3402). The BLAST programs identify homologous sequences by identifying similar segments, which are referred to herein as “high-scoring segment pairs,” between a query amino or nucleic acid sequence and a test sequence which is preferably obtained from a protein or nucleic acid sequence database. Preferably, the statistical significance of a high-scoring segment pair is evaluated using the statistical significance formula (Karlin and Altschul, 1990), the disclosure of which is incorporated by reference in its entirety. The BLAST programs can be used with the default parameters or with modified parameters provided by the user.

### Methods

In a second aspect, the present invention provides methods of detecting SARS-CoV-2 in a sample. The methods comprise: (a) amplifying the SARS-CoV-2 target nucleic acid in the sample; (b) contacting the amplified nucleic acid with an aptasensor disclosed herein and the cognate ligand of its aptamer; and (c) detecting any signal produced by the aptamer binding to its cognate ligand. In these methods, detection of the signal indicates that SARS-CoV-2 is present in the sample.

Any sample can be tested for the presence of SARS-CoV-2 using the methods described herein. In some embodiments, the sample is obtained from a subject, e.g., a human or animal subject. In such cases, the sample may comprise saliva, a nasopharyngeal swab, blood, serum, or sputum. Other suitable samples include, without limitation, food samples, drinking water, environmental samples, agricultural products, plastic and packaging materials, paper, clothing fibers, and metal surfaces. In certain embodiments, the methods are used in food safety and biosecurity applications, such as screening food products and materials used in food processing or packaging for the presence of the virus. In some embodiments, the sample is heat inactivated (e.g., at 65° C.) or frozen (e.g., at −80° C.) prior to testing.

In the first step of the present methods, the SARS-CoV-2 target nucleic acid is amplified. Amplification may be performed using any known nucleic acid amplification method. In some embodiments, the amplification step is performed using a PCR-based method. Suitable PCR-based methods include, without limitation, standard PCR, quantitative PCR (qPCR), PCR-restriction fragment length polymorphism (PCR-RFLP), asymmetrical PCR, transcript mediated amplification (TMA), self-sustained sequence replication (3SR), and ligase chain reaction (LCA). In preferred embodiments, the amplification step is performed using an isothermal amplification method. Suitable isothermal amplification methods include, without limitation, nucleic acid sequence-based amplification (NASBA), loop-mediated isothermal amplification (LAMP), reverse transcription loop-mediated isothermal amplification (RT-LAMP), strand displacement amplification (SDA), recombinase polymerase amplification (RPA), reverse transcription recombinase polymerase amplification (RT-RPA), helicase-dependent amplification (HDA), reverse transcription helicase-dependent amplification (RT-HDA), nicking enzyme amplification reaction (NEAR), signal mediated amplification of RNA technology (SMART), rolling circle amplification (RCA), isothermal multiple displacement amplification (IMDA), single primer isothermal amplification (SPIA), and polymerase spiral reaction (PSR). In some embodiments, the amplification method involves performing reverse transcription and transcription in a single reaction. In the Examples, the inventors provide aptasensors that can be used to detect amplicons generated using the isothermal amplification methods NASBA, RT-RPA, and RT-LAMP. Thus, in some embodiments, the amplification method is selected from NASBA, RT-RPA, or RT-LAMP.

To allow for detection using the aptasensors disclosed herein, the amplification method must produce a single-stranded product. Some amplification methods, such as NASBA and LAMP, produce single-stranded regions that are suitable for binding. However, methods that produce a double-stranded DNA (dsDNA) product must be adapted, e.g., by supplying a higher concentration of one of the primers (akin to asymmetric PCR) or by adding a T7 promoter that facilitates transcription of dsDNA products into ssRNA.

At a minimum, the amplicons detected using the aptasensors disclosed herein should be at about 10 nucleotides in length, excluding primer binding sites. For example, an amplicon that is 12 nucleotides in length can hybridize with an aptasensor comprising a 4 nucleotide toehold, a 6 nucleotide b domain, and 2 nucleotide c domain, assuming that the melting temperature is at least room temperature.

In the second step of the present methods, the amplified nucleic acid is contacted with an aptasensor disclosed herein and the cognate ligand of its aptamer. The cognate ligand used with the present invention may be any ligand that generates a detectable signal upon binding to the aptamer portion of the aptasensor. Suitable cognate ligands include, without limitation, -Difluoro-4-Hydroxybenzylidene)-2-Methyl-1-(2,2,2-Trifluoroethyl)-1H-Imidazol-5(4 H)-One (DFHBI-1T), 3,5-difluoro-4-hydroxybenzylidene-imidazolinone-2-oxime (DFHO), (Z)-3-((1H-benzo[d]imadazol-4-yl)methyl)-5-(3,5-difluoro-4-hydroxybenzylidene)-2-methyl-3,5-dihydro-4H-imidazol-4-one] (BI), and 3,5-difluoro-4-hydroxybenzylidene-imidazolinone-2-oxime-1-benzoimidazole (OBI).

In the final step of the present methods, any signal produced by the aptamer binding to its cognate ligand is detected. The detection method used in this step may be quantitative (i.e., measure the amount of the SARS-CoV-2 target nucleic acid present in the sample) or qualitative (i.e., simply determine whether the SARS-CoV-2 target nucleic acid is present in the sample at a detectable level). In embodiments in which the detectable signal is a colorimetric signal, detection may be performed by eye or using a spectrophotometer. In embodiments in which the detectable signal is a fluorescent signal, detection may be performed using a fluorescence instrument, such as a fluorometer, fluorospectrometer, or fluorescence spectrometer.

Alternatively, a fluorescent signal may be detected using a simple electronic reader comprising readily available components, as is described in Example 1 in the section titled “Detection equipment”. For example, the electronic reader may measure a fluorescence signal produced from a reaction that is placed into the reader between a light source (i.e., that supplies light of an appropriate wavelength to excite a fluorophore cognate ligand) and electronic sensors (i.e., that detect any emission produced by an excited fluorophore cognate ligand). In some cases, the light source is a light emitting diode (LED) light source. In some cases, the electronic reader may be configured to measure the output of a freeze-dried, paper-based reaction. In other cases, it may be configured to measure the output of a liquid reaction. In some cases, the output is read using onboard electronics that provide low-noise measurements of signal changes.

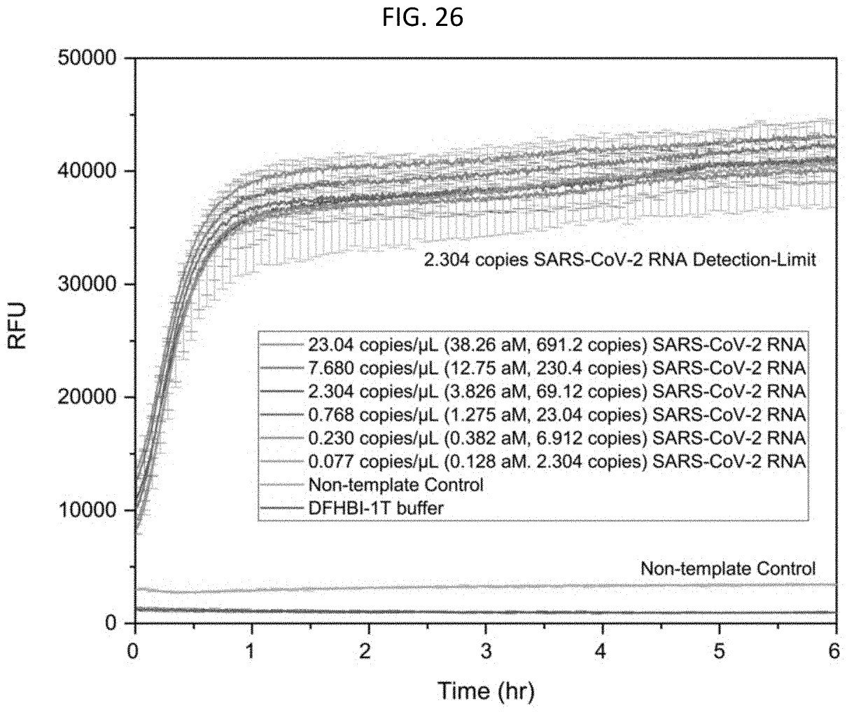

In the Examples, the inventors demonstrate that the methods of the present invention are highly sensitive. For instance, the inventors achieved a limit of detection of 2 copies of SARS-CoV-2 per 30-μL reaction or 0.13 aM using RT-LAMP primers amplifying the spike gene of SARS-CoV-2 and a single Broccoli aptasensor (see FIG. 26). Thus, in some embodiments, the SARS-CoV-2 target nucleic acid is detectable at a concentration as low as 0.13 aM.

Additionally, the inventors demonstrate that their aptasensors rapidly produce a detectable signal upon binding to a SARS-CoV-2 target nucleic acid. Thus, in some embodiments, the signal, if present, is detectable in less than 1 hour. In some embodiments, the signal, if present, is detectable in less than 50 minutes, less than 40 minutes, less than 30 minutes, less than 20 minutes, less than 15 minutes, or less than 10 minutes.

To verify that the sample has been correctly processed for use in these methods and reduce false negative results, it may be advantageous to use a positive control. Thus, in some embodiments, the methods further comprise amplifying a control nucleic acid in the sample and detecting the amplified control nucleic acid. The “control nucleic acid” may be any nucleic acid that is expected to be present in all of the samples tested. For example, when a sample is from a human patient, a human gene product can be used as a control nucleic acid. Following amplification, the control nucleic acid can be detected using any means of nucleic acid detection known in the art. Suitable methods for detecting nucleic acids include, without limitation, ethidium bromide staining, quantitative PCR, fluorometer detection, sequencing, and the like.

In some embodiments, the control nucleic acid is detected using an aptasensor. In the Examples, the inventors tested aptasensors that detect the control nucleic acids human RNase P mRNA, beta actin (ACTB) mRNA, and 18S rRNA. The sequences of these “control aptasensors” are provided in Tables 11, 13, 14, 20, 22, and 23 as SEQ ID NOs: 119-120, 137-150, and 249-256. Thus, in some embodiments, the control nucleic acid is selected from the group consisting of: human RNase P mRNA, beta actin (ACTB) mRNA, and 18S rRNA. In some embodiments, the control nucleic acid is detected using an aptasensor comprising a sequence selected from SEQ ID NOs: 119-120, 137-150, and 249-256.

In some embodiments, the methods involve the detection of two or more different nucleic acids (e.g., one or more SARS-CoV-2 target nucleic acids and, optionally, one or more control nucleic acids). This can be accomplished using a two-channel assay that utilizes two or more different aptamer-ligand pairs with different spectral properties, as described in Example 2.

The RNA genome of SARS-CoV-2 may not be accessible in an unprocessed sample. Thus, in the embodiments, the methods further comprise isolating, purifying, or extracting RNA prior to step (a). Suitable extraction methods for isolating viral RNA from saliva samples include, without limitation, protease K treatment, Triton X-100 processing, and use of ARCIS reagents. In some embodiments, the extraction is performed using a commercially available kit (e.g., PureLink RNA extraction kit). In preferred embodiments, any virus present in the sample is heat inactivated prior to step (a). Heat inactivation serves the dual-purpose of extracting the viral genome from virions and killing the virus, making this method safer to perform outside of a biosafety level 3 laboratory. Heat inactivation is performed by heating the sample to a temperature sufficient to kill the virus and to release its genomic RNA. For example, heat inactivation may be performed by subjecting the sample to a high temperature for at least about 3 minutes, about 5 minutes, about 10 minutes, about 20 minutes, or about 30 minutes. The temperature used for heat inactivation is preferably between about 60° C. and 150° C., and more preferably between about 60° C. and 100° C. In some embodiments, the inactivating step comprises heating the sample to about 65° C. for about 30 minutes. In other embodiments, the inactivating step comprises heating the sample to about 98° C. for about 5 minutes.

In some embodiments, the methods are adapted for high-throughput and/or rapid detection. For example, the method may utilize a high-throughput format, such as a multi-well plate (e.g., a 6-, 12-, 24-, 48-, 96-, 384-, or 1536-well plate). For convenience, the multi-well plate may be pre-aliquoted with a master mix for the amplification reaction (e.g., RT-LAMP enzyme, buffer, and primers) or for the aptasensor readout reaction (e.g., aptasensor RNA, cognate ligand, and buffer), as described in Example 2. The method may also utilize a device configured for rapid detection in a clinical setting or in the field. Such devices may comprise, for example, a preserved paper test article or test tubes comprising the aptasensor. In some embodiments, the aptasensor is freeze-dried (e.g., on a paper test article or in a test tube) to render it stable at room temperature. Kits

In a third aspect, the present invention provides kits for detecting SARS-CoV-2 comprising the aptasensors disclosed herein. Optionally, the kits can further include instructions and/or additional reagents for performing the SARS-CoV-2 detection methods described herein.

In some embodiments, the kits further comprise primers that can be used to specifically amplify the SARS-CoV-2 target nucleic acid. The primers in the kit may be suitable for use with any amplification method. In some embodiments, the primers in the kit are designed for use in an isothermal amplification method, such as NASBA, LAMP, RT-LAMP, RPA, RT-RPA, HDA, or RT-HDA.

In some embodiments, the kits further comprise reagents that allow for the detection of a control nucleic acid, such as primers that specifically amplify the control nucleic acid and/or an aptasensor that binds to the control nucleic acid.

The present disclosure is not limited to the specific details of construction, arrangement of components, or method steps set forth herein. The compositions and methods disclosed herein are capable of being made, practiced, used, carried out and/or formed in various ways that will be apparent to one of skill in the art in light of the disclosure that follows. The phraseology and terminology used herein is for the purpose of description only and should not be regarded as limiting to the scope of the claims. Ordinal indicators, such as first, second, and third, as used in the description and the claims to refer to various structures or method steps, are not meant to be construed to indicate any specific structures or steps, or any particular order or configuration to such structures or steps. All methods described herein can be performed in any suitable order unless otherwise indicated herein or otherwise clearly contradicted by context. The use of any and all examples, or exemplary language (e.g., “such as”) provided herein, is intended merely to facilitate the disclosure and does not imply any limitation on the scope of the disclosure unless otherwise claimed. No language in the specification, and no structures shown in the drawings, should be construed as indicating that any non-claimed element is essential to the practice of the disclosed subject matter. The use herein of the terms “including,” “comprising,” or “having,” and variations thereof, is meant to encompass the elements listed thereafter and equivalents thereof, as well as additional elements. Embodiments recited as “including,” “comprising,” or “having” certain elements are also contemplated as “consisting essentially of” and “consisting of” those certain elements.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure. Use of the word “about” to describe a particular recited amount or range of amounts is meant to indicate that values very near to the recited amount are included in that amount, such as values that could or naturally would be accounted for due to manufacturing tolerances, instrument and human error in forming measurements, and the like. All percentages referring to amounts are by weight unless indicated otherwise.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

The following examples are meant only to be illustrative and are not meant as limitations on the scope of the invention or of the appended claims.

EXAMPLES

Example 1

In the following Example, the inventors describe their rapid, low-cost, highly sensitive method for detection of SARS-CoV-2. As illustrated in FIG. 2, their method uses aptasensors that bind to specific SARS-CoV-2 gene sequences or the complement thereof.

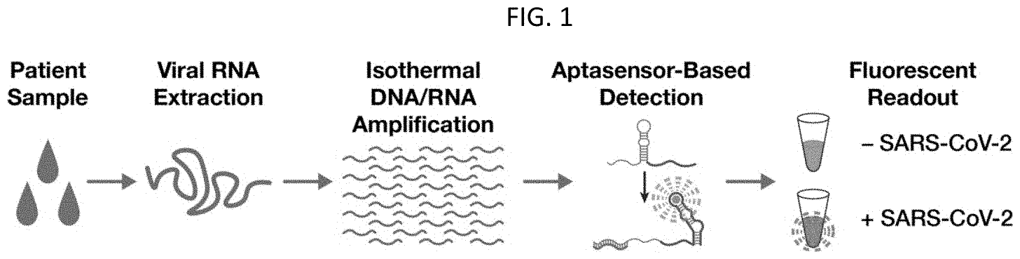

A general schematic depicting this method is shown in FIG. 1. A patient sample, obtained from saliva, nasopharyngeal swab, blood, or another matrix, is first heated at 65° C. for 30 minutes. This treatment not only inactivates the virus to enable safe handling, but it also releases genomic RNA from the SARS-CoV-2 virions. The released RNA is then amplified using an isothermal amplification method. The sequences of the amplified nucleic acids are then verified using toehold-based aptasensors that produce a strong fluorescence signal when they bind to the target SARS-CoV-2 sequence. Specifically, the inventors provide aptasensors that have been designed to detect amplicons generated by nucleic acid sequence-based amplification (NASBA), recombinase polymerase amplification (RPA), and loop-mediated isothermal amplification (LAMP)). Because isothermal amplification methods can produce non-specific amplification products, use of these aptasensors also reduces the potential for false positive results. Fluorescence signals from the aptasensors can be read out using a plate reader, smart phone camera, or by eye.

### Aptasensor Design

Libraries of aptasensors targeting multiple regions of the SARS-CoV-2 genome were designed for use in this method. The main target regions for these sensors are shown in FIG. 2A and include regions (typically about 35 nucleotides in length) within the SARS-CoV-2 genes Orflb, RdRP, E, and N. Aptasensors were designed to target either the gene region itself (i.e., the sense sequence) or the complement of the gene region (i.e., the antisense sequence), as either the sense or antisense gene sequence can be generated depending on the amplification method used. Two different aptamers, i.e., Broccoli and Corn, were used in the aptasensors.

FIG. 2B shows the operating mechanism of the aptasensors comprising the Broccoli aptamer. In these sensors, the Broccoli aptamer sequence is placed at the 3′ end of the transcript. However, aptamer formation is unable to occur as a result of a strong hairpin secondary structure programmed in the 5′ portion of the transcript. Importantly, this hairpin structure encloses the main stem of the aptamer, comprised of a domain b and its reverse complement b*. The hairpin is further extended by a stem domain c/c* that is used to stabilize the hairpin structure and prevent spontaneous aptamer formation. When the target SARS-CoV-2 RNA is present, a toehold domain a of the aptamer binds to the target RNA with the sequence c*-b*-a*. Binding through the toehold enables the target RNA to unwind the stem of the aptasensor and expose the downstream sequences c* and b*. The newly released b* domain can in turn bind to the b domain at the 3′ end of the aptasensor enabling formation of the Broccoli aptamer structure. The Broccoli aptamer is then free to bind to the fluorogen DFHBI-1T ((Z)-4-(3,5-difluoro-4-hydroxybenzylidene)-2-methyl-1-(2,2,2-trifluoroethyl)-1H-imidazol-5(4 H)-one). Binding to Broccoli activates the fluorescence of DFHBI-1T, providing a strong green emission signal under blue light illumination. Notably, the sequence of the b domain that defines the stem of the Broccoli output aptamer is largely sequence independent, which enables the aptasensor to detect virtually any target RNA sequence.

FIG. 2C shows the operating mechanism of the aptasensors comprising the Corn aptamer. In the Corn aptasensors, a hairpin in the 5′ region of the sensor is used to prevent formation of the Corn aptamer at the 3′ end of the transcript. The b domain defines the critical base stem of the Corn aptamer and in this case requires G or C base at the locations indicated in FIG. 2C. Upon binding to the target RNA, the toehold-mediated strand-displacement reaction releases domains c* and b* to promote formation of the active Corn aptamer. The assembled aptamer can then bind to the fluorogen DFHO (4-(3,5-difluoro-4-hydroxybenzylidene)-1-methyl-5-oxo-4,5-dihydro-1H-imidazole-2-carbaldehyde oxime), which then produces a strong yellow fluorescence emission under green or blue light illumination.

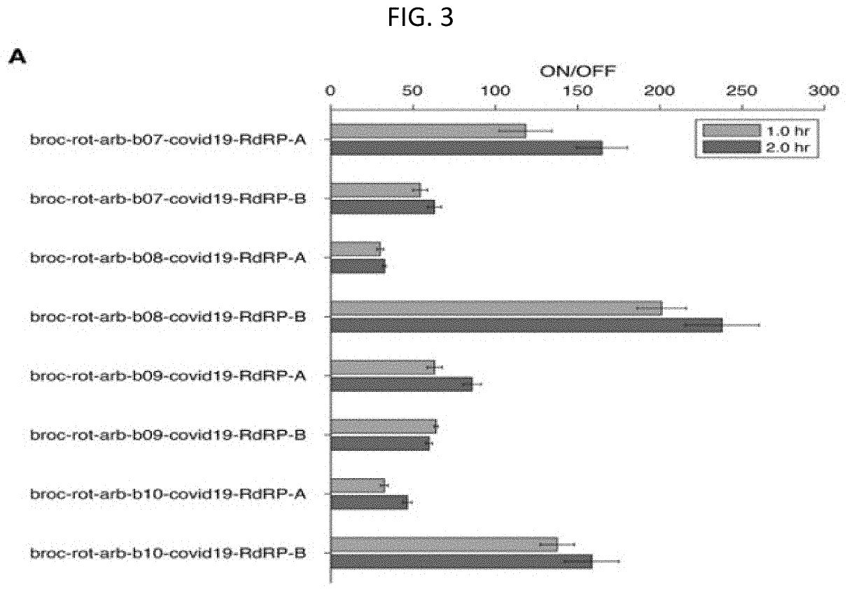

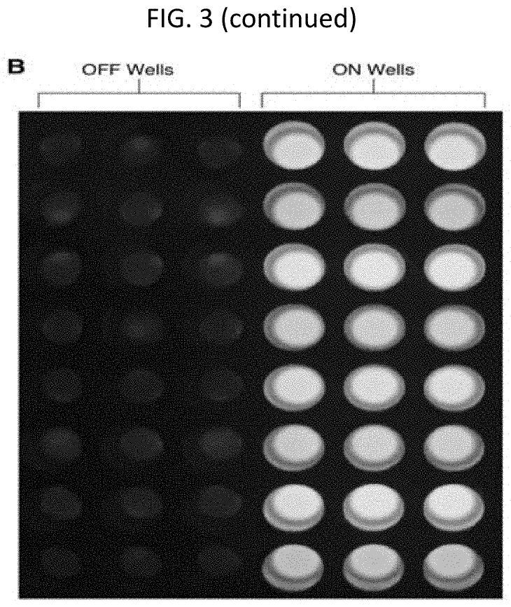

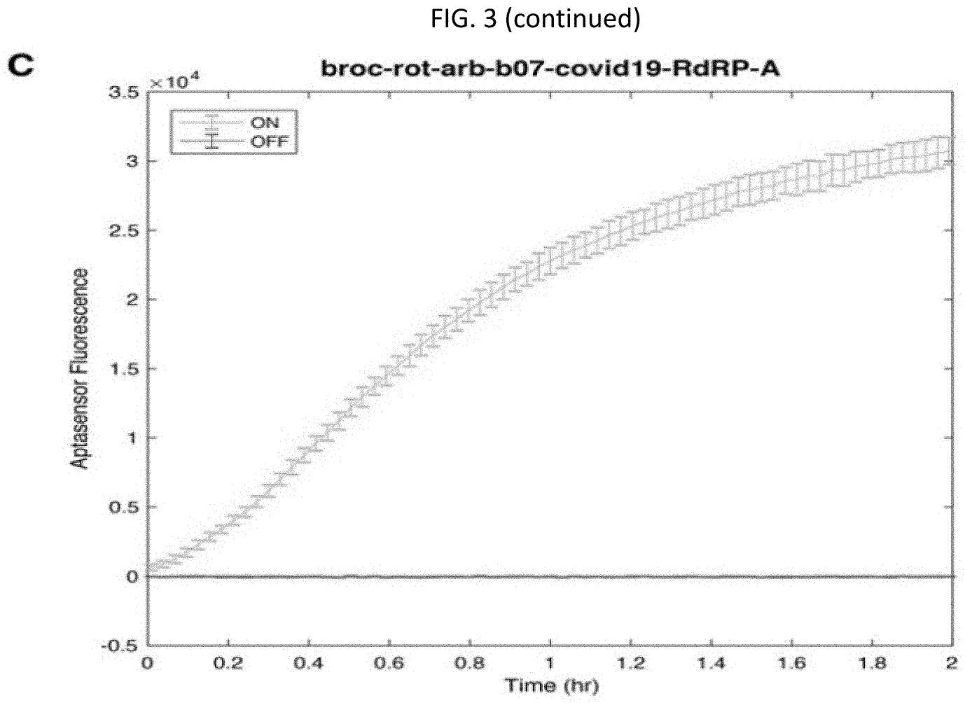

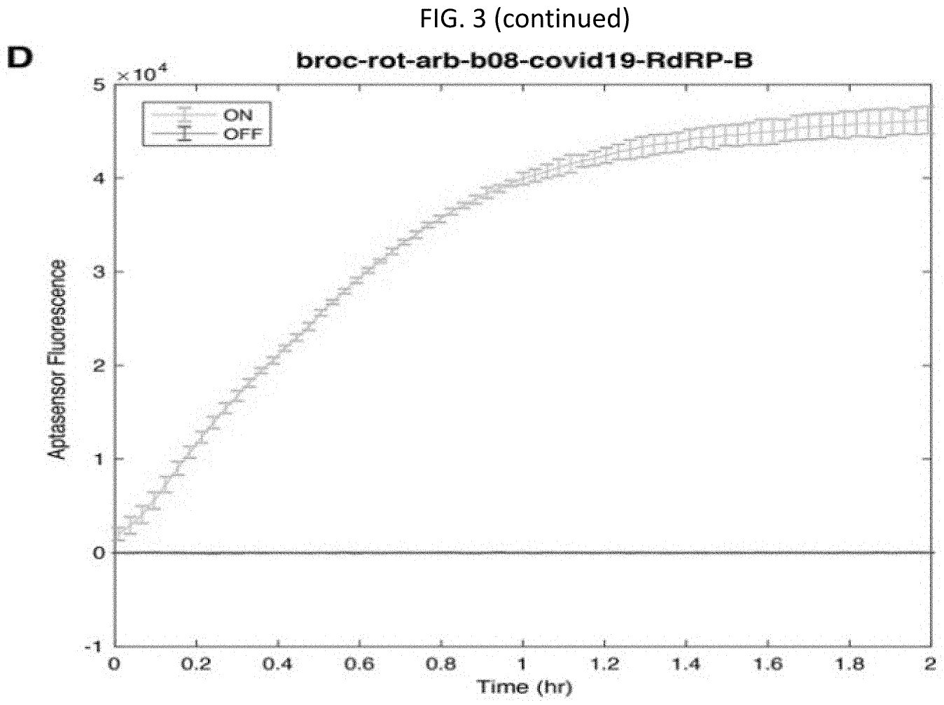

Aptasensors based on the Broccoli and Corn aptamers were designed computationally using a custom algorithm. The resulting sensor transcripts were then screened for function by challenging them with synthetic versions of the SARS-CoV-2 genomic targets after in vitro transcription. FIG. 3 shows the performance of a library of eight Broccoli aptasensors targeting the sense orientation of the SARS-CoV-2 RdRP gene (SEQ ID NOs:1-8; see Table 1). Three out of the eight aptasensors provide a large ≥100-fold increase in fluorescence upon detection of the cognate target compared to reactions without the target RNA (FIG. 3A). To observe the fluorescence of the sensors, the reactions were illuminated in a microplate using a blue-light transilluminator system equipped with an orange optical filter. A photograph of the aptasensors after the reactions is shown in FIG. 3B. The sensors were tested in triplicate with and without the target RNA. Clearly visible is the strong green fluorescence produced by the aptasensors containing the SARS-CoV-2 target RNA. Time-course measurements of the sensors are provided in FIG. 3C, 3D to show the activation speed of the sensors in 37° C. reactions. These data show that statistically significant fluorescence signals can be detected at the start of the reaction in a plate reader and strong activation is observed within 15 minutes.

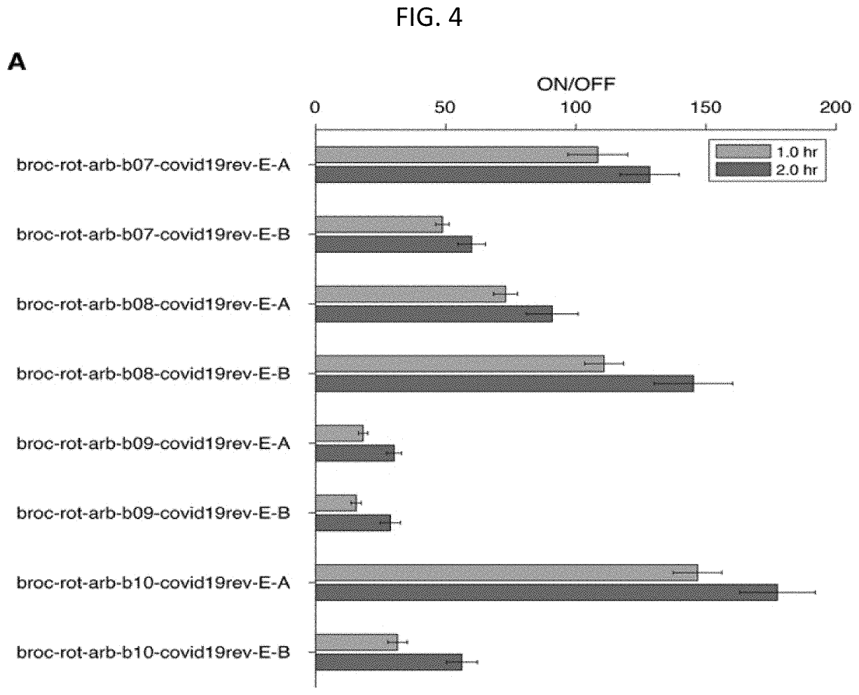

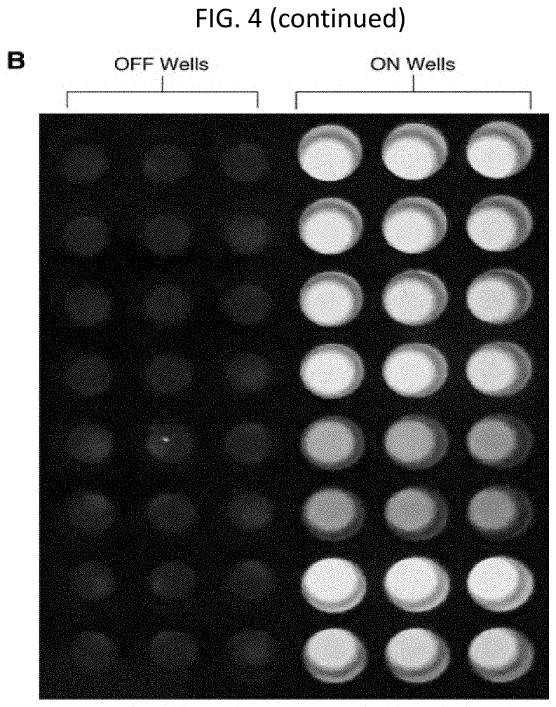

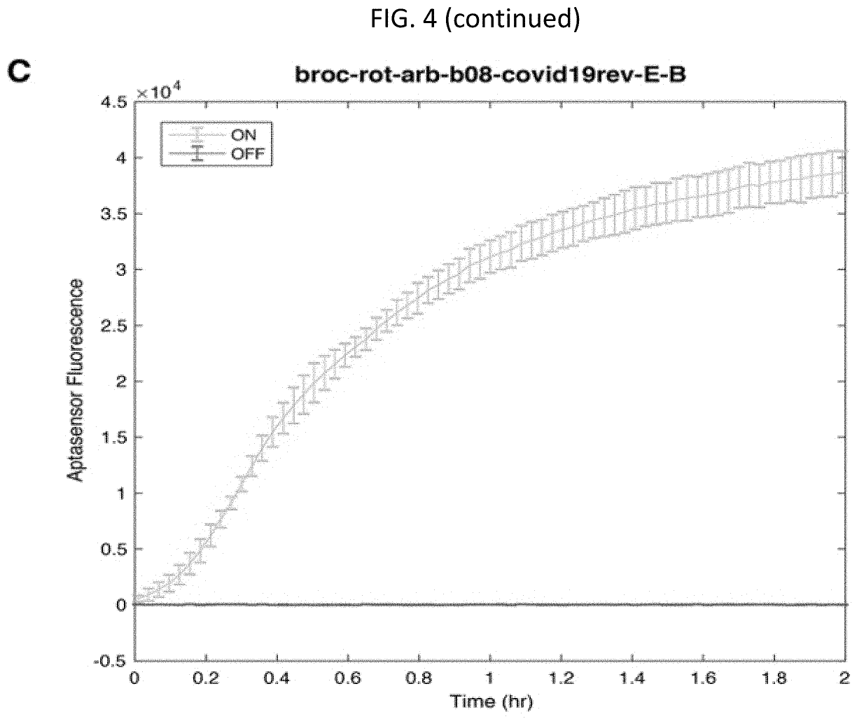

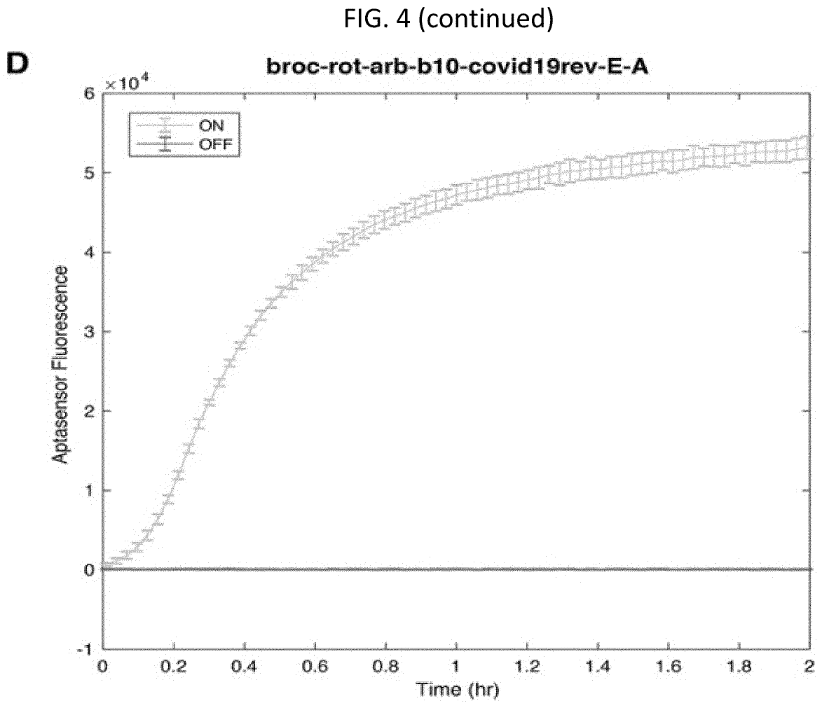

FIG. 4 shows the performance evaluation for a library of eight Broccoli aptasensors targeting the sense orientation of the SARS-CoV-2 E gene (SEQ ID NOs:2-16; see Table 2). Three out of the eight devices tested provide at least a 100-fold ON/OFF ratio (FIG. 3A) and provide strong visible green fluorescence under illumination (FIG. 3B). Like the RdRP aptasensors, these systems activate very quickly with strong fluorescence occurring within 15 minutes (FIG. 4C, 4D).

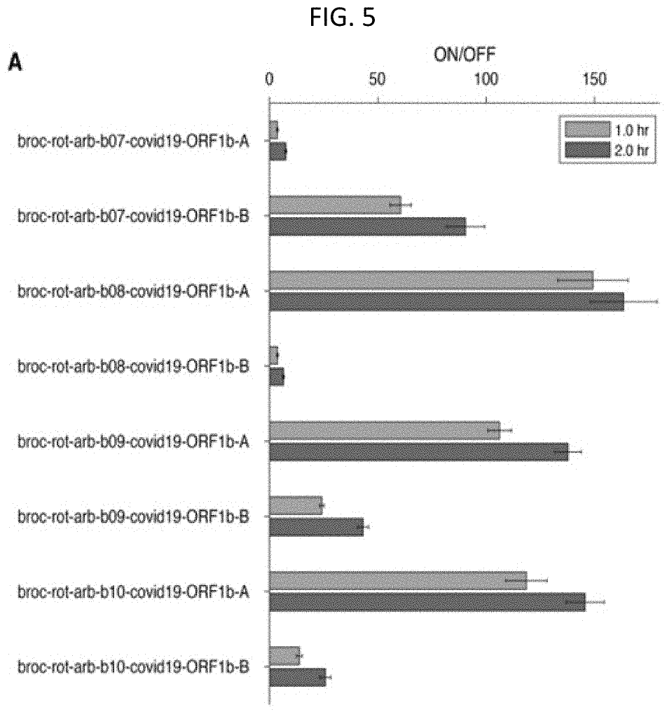

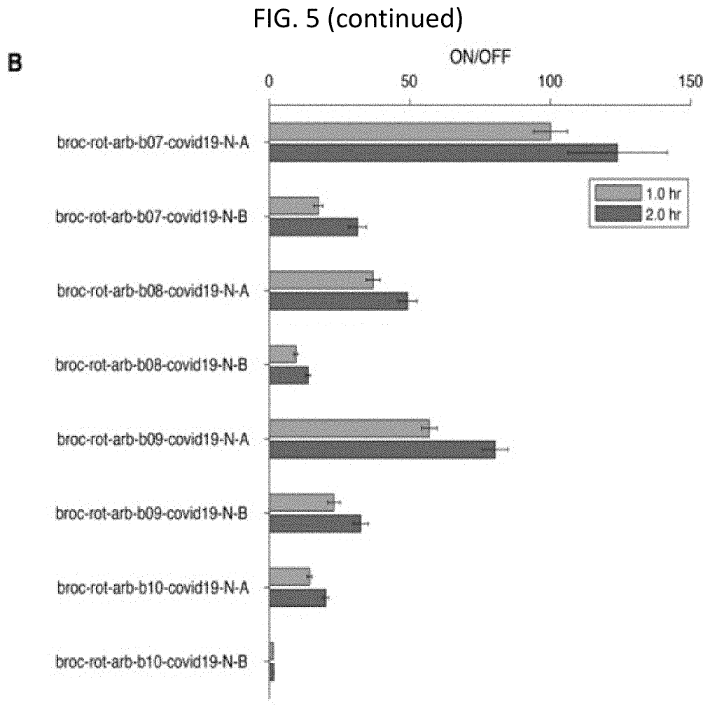

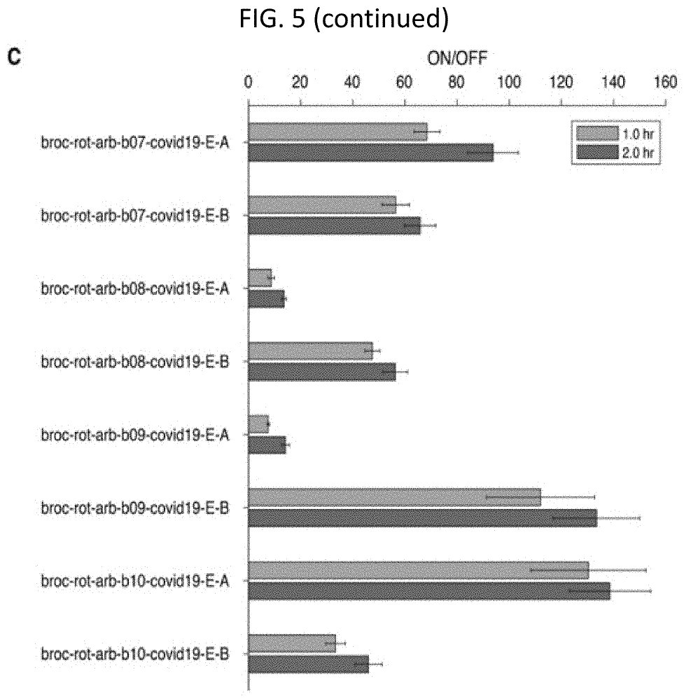

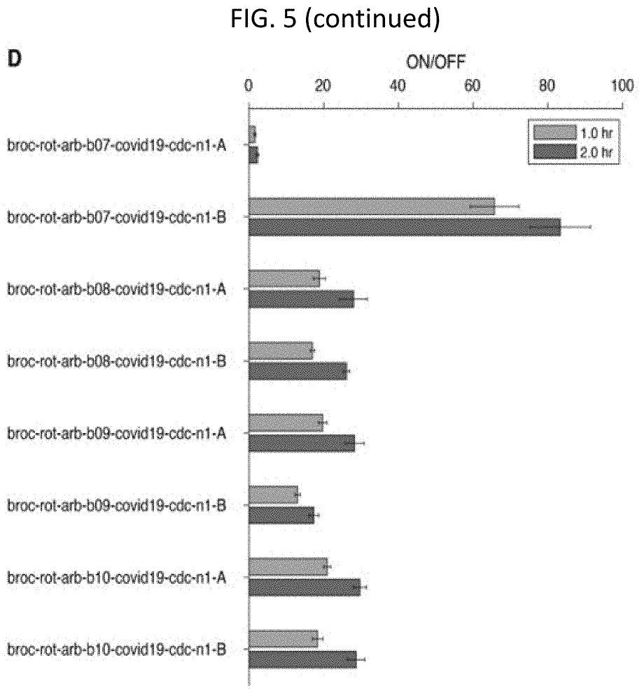

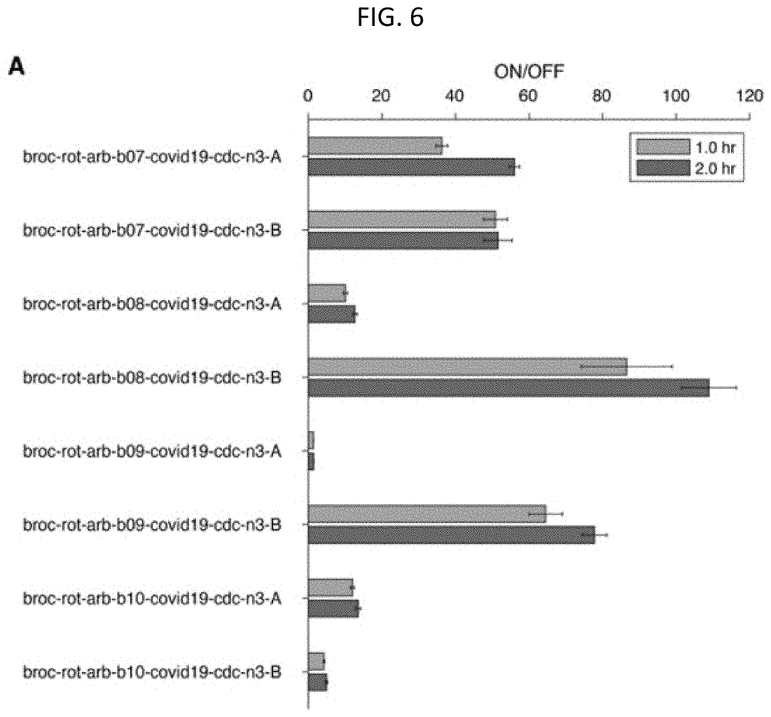

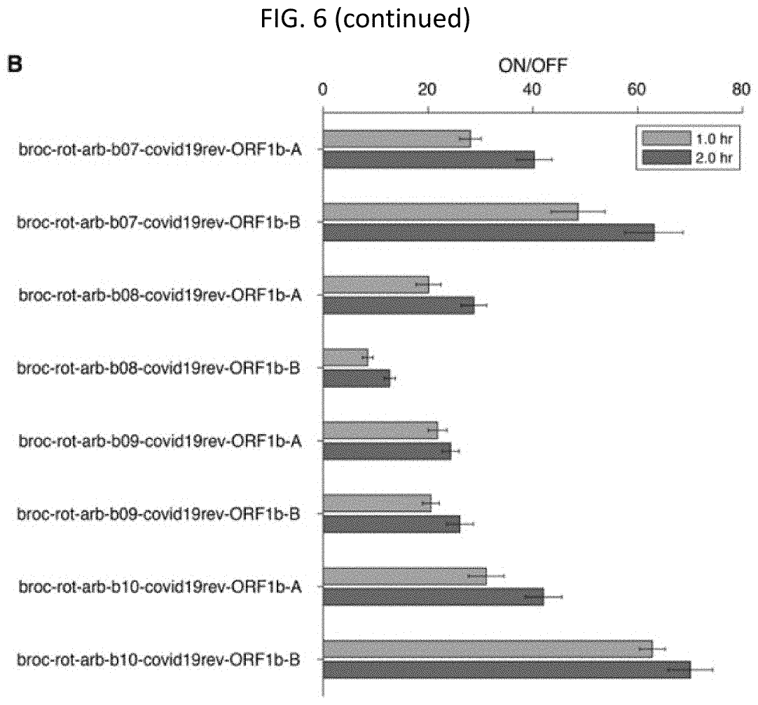

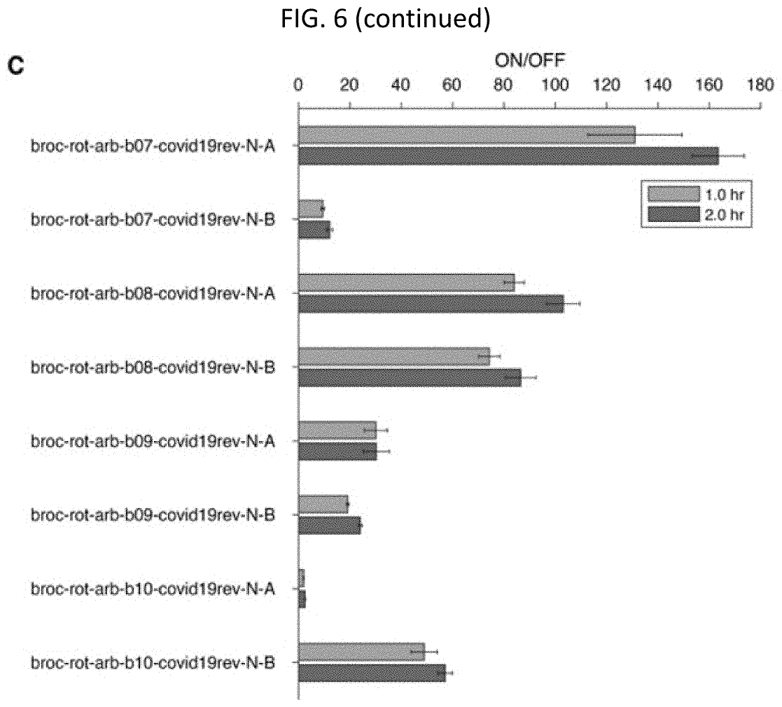

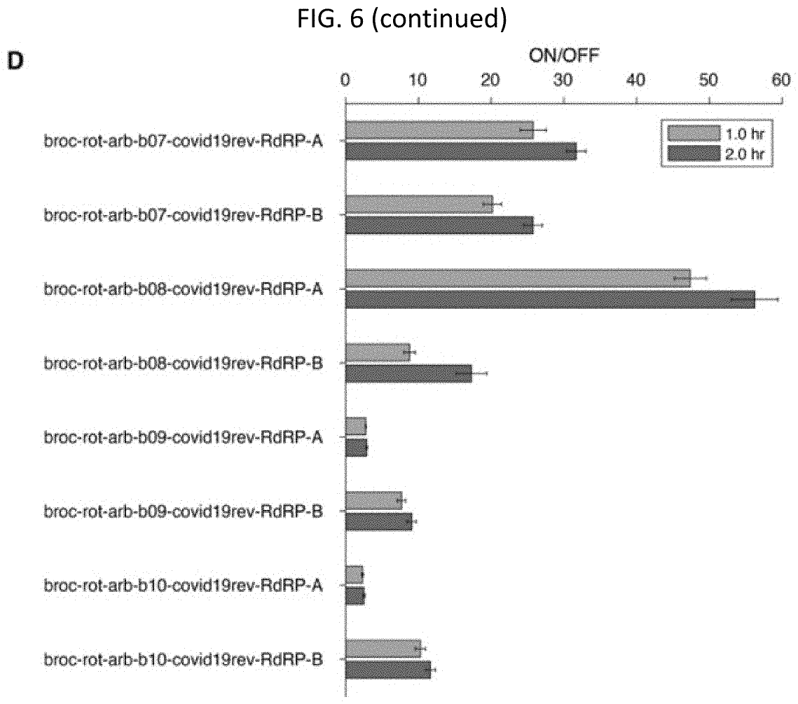

Tests were also conducted with multiple other libraries of Broccoli aptasensors targeting different regions of the SARS-CoV-2 genome, including: (A) the sense orientation of Orflb, (B) the sense orientation of the N gene, (C) the sense orientation of the E gene, and (D) the sense orientation of the N gene. ON/OFF ratios for libraries of aptasensors targeting Orflb, the N gene, and E gene are shown in FIG. 5 (SEQ ID NOs:17-48; see Table 3). Data from aptasensors targeting the N gene, Orflb, and RdRP are provided in FIG. 6 (SEQ ID NOs:49-80; see Table 4).

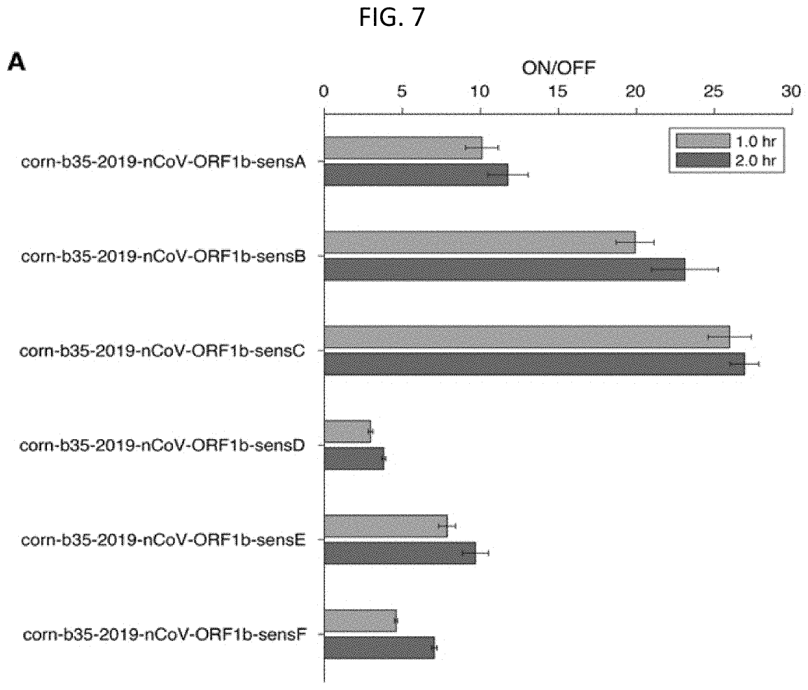

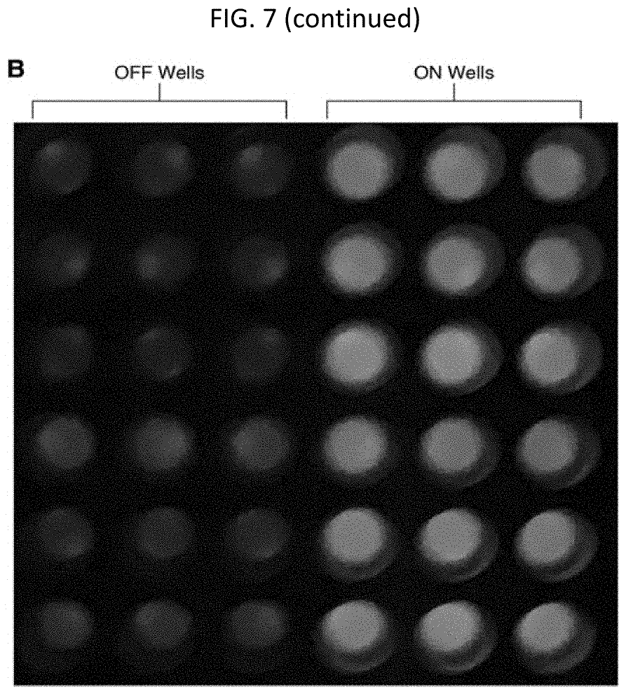

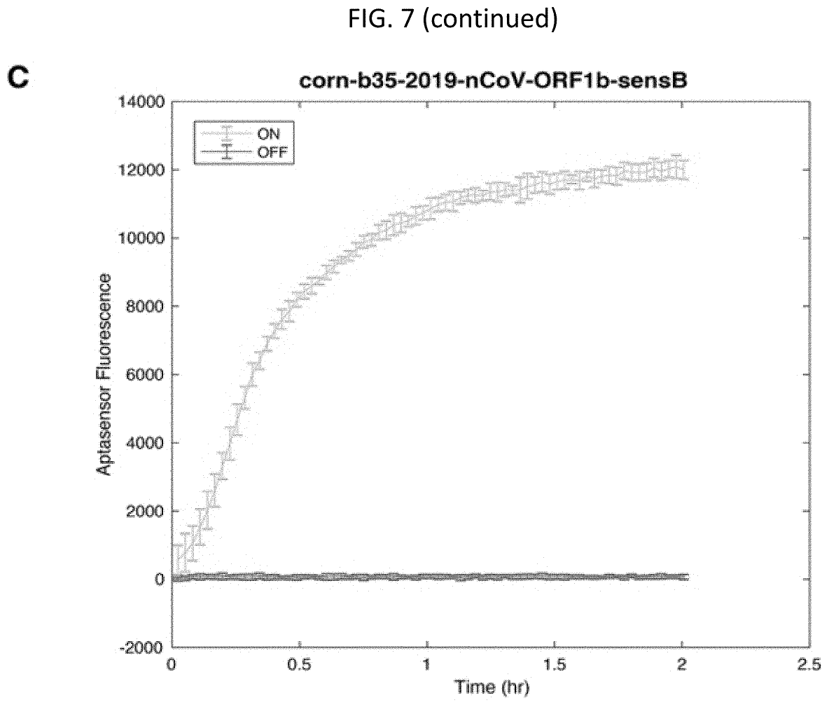

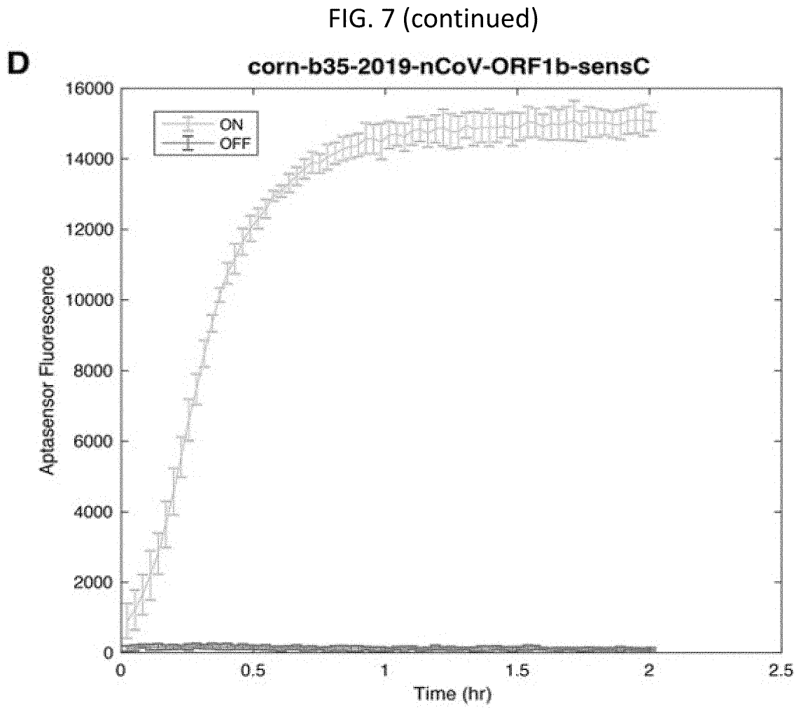

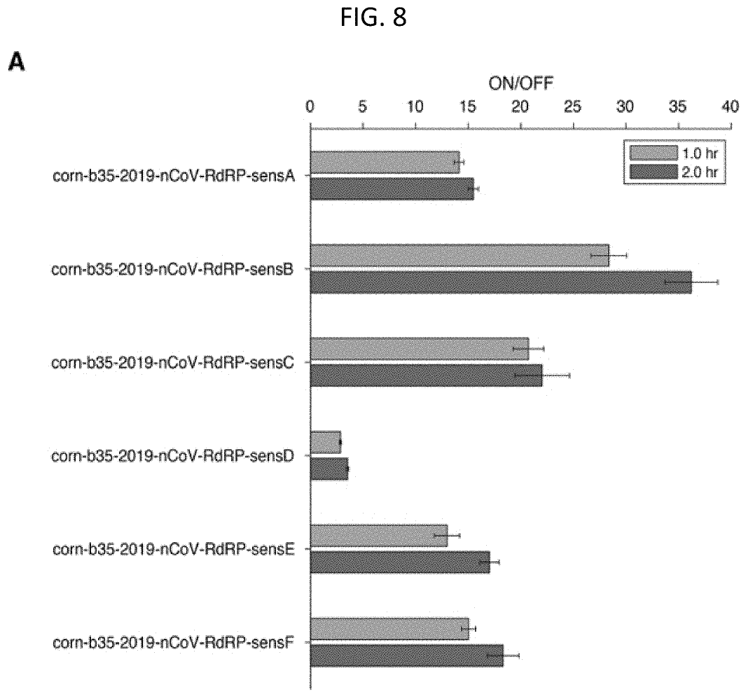

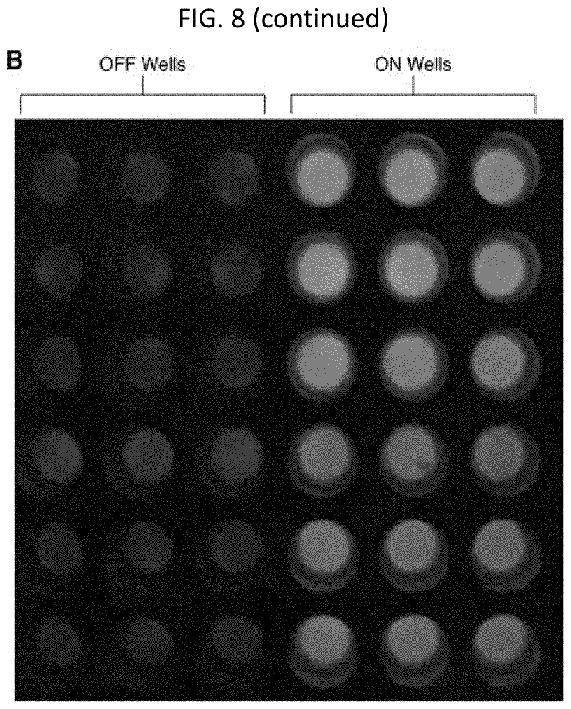

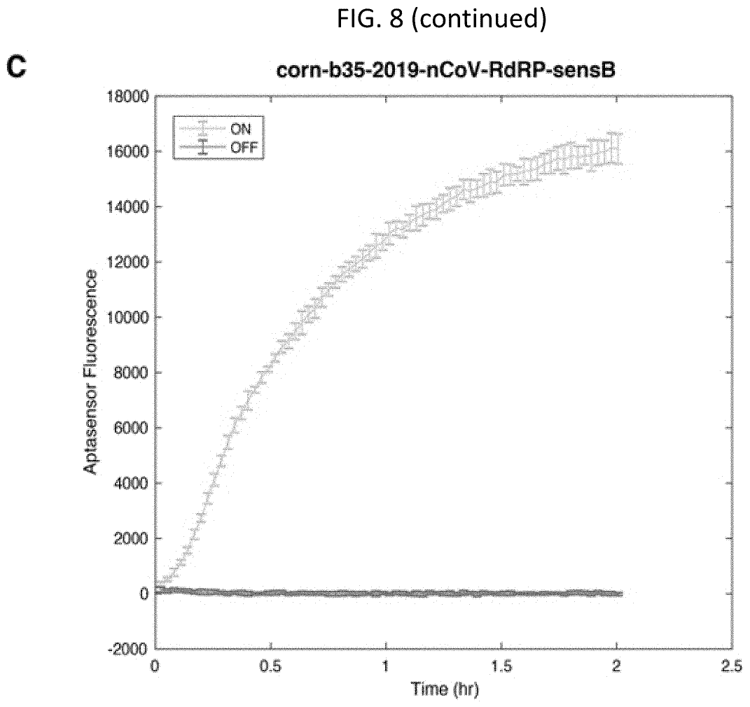

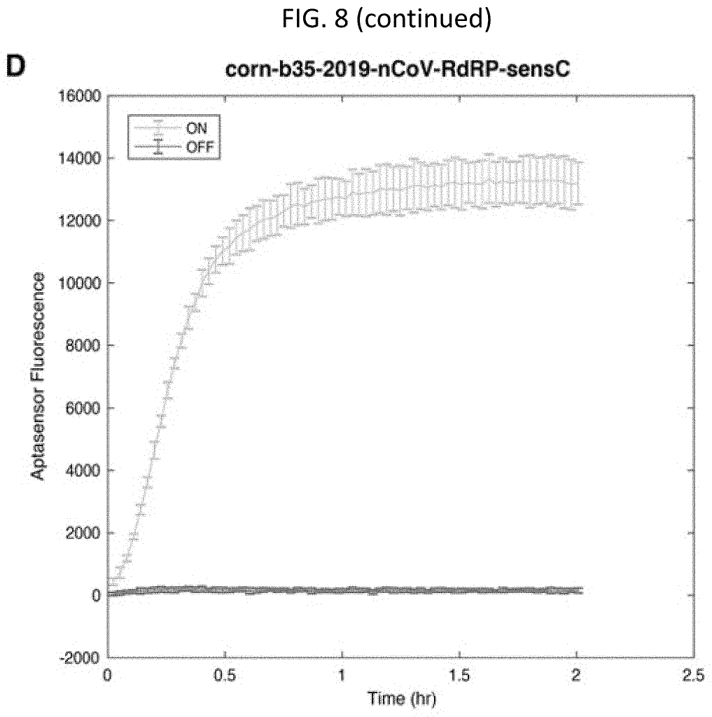

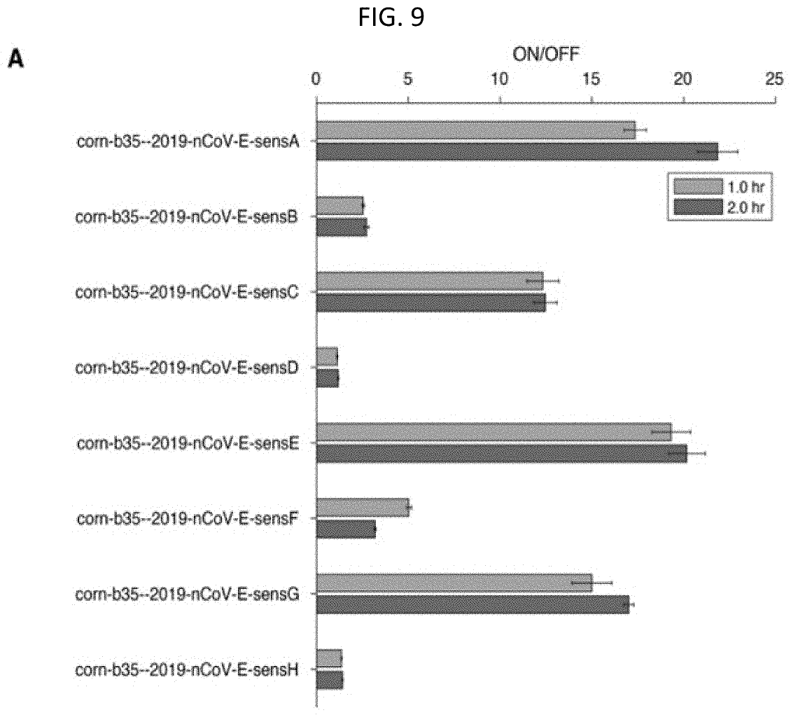

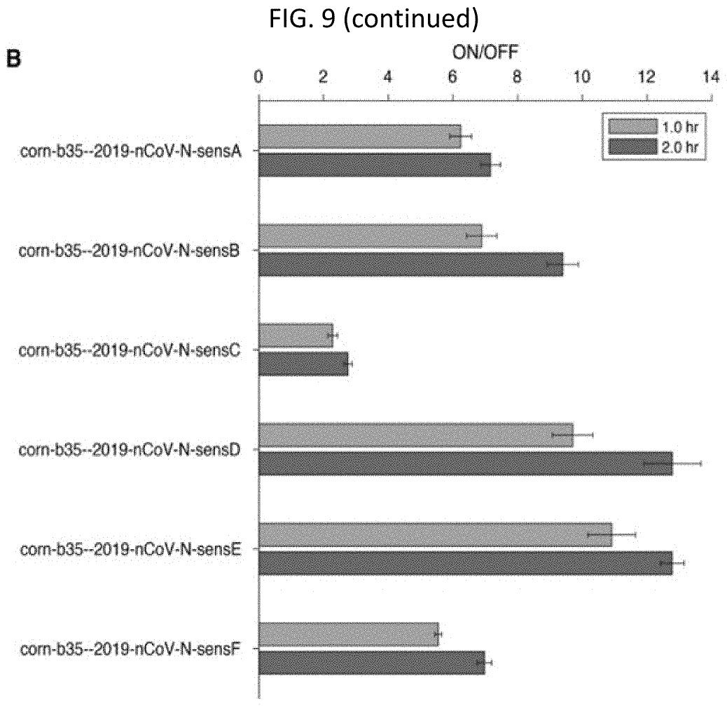

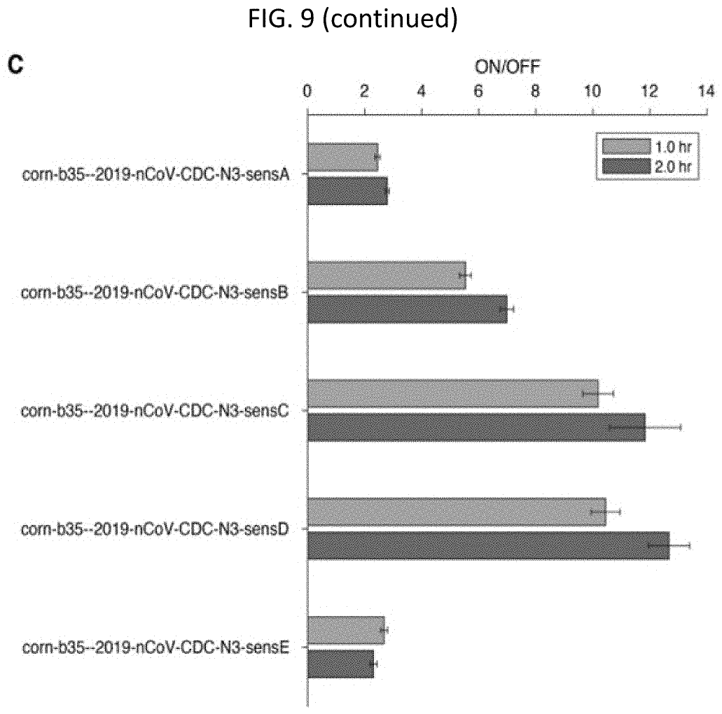

Results from aptasensors based on the Corn aptamer are shown in FIGS. 7 to 9. In general, these Corn aptasensors display lower ON/OFF ratios compared to the Broccoli-based systems as they emit weaker fluorescence output. A set of six aptasensors targeting the sense orientation of the Orflb region of SARS-CoV-2 provided ON/OFF ratios up to ˜26-fold in the presence of synthetic target RNAs (FIG. 7A) (SEQ ID NOs:81-86; see Table 5). These aptasensors also provided clearly visible yellow fluorescence in triplicate reactions (FIG. 7B) using the same blue-light transilluminator system used for visualizing the Broccoli aptasensors. The top-performing Corn aptasensors for Orflb also activated very rapidly providing a strong fluorescence signal within 15 minutes of the start of the reaction (FIG. 7C, 7D). A second library of six Corn aptasensors targeting the RdRP gene of SARS-CoV-2 yielded five sensors with ON/OFF ratios above 15-fold (FIG. 8A) (SEQ ID NOs:87-92; see Table 6). The best of these sensors exhibited a —35-fold increase in signal with the synthetic target RNA after two hours of reaction. Photographs of these aptasensors also demonstrate their strong yellow fluorescent output (FIG. 8B). Time-course measurements in FIG. 8C, 8D show rapid sensor activation with a strong signal obtained within —10 minutes for the sensor with the highest ON/OFF ratio. Data from an additional three libraries is shown in FIG. 9 (SEQ ID NOs:93-110; see Table 7). These results show that the Corn aptasensors can routinely reach ON/OFF ratios above 10 and can be applied to the targets from the E gene and N gene of SARS-CoV-2.

### Aptasensors for Detection of Nucleic Acid Sequence-Based Amplification (NASBA) Amplicons

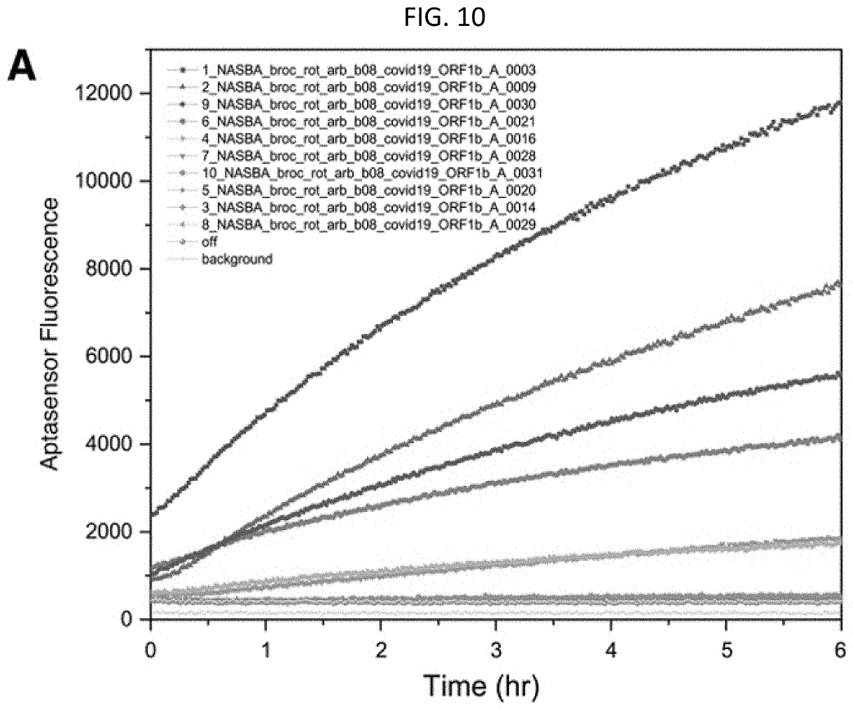

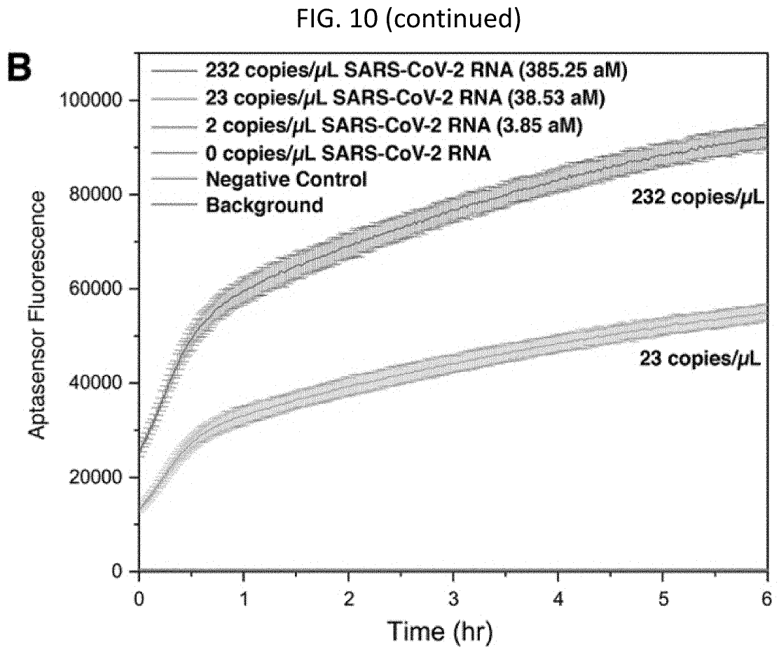

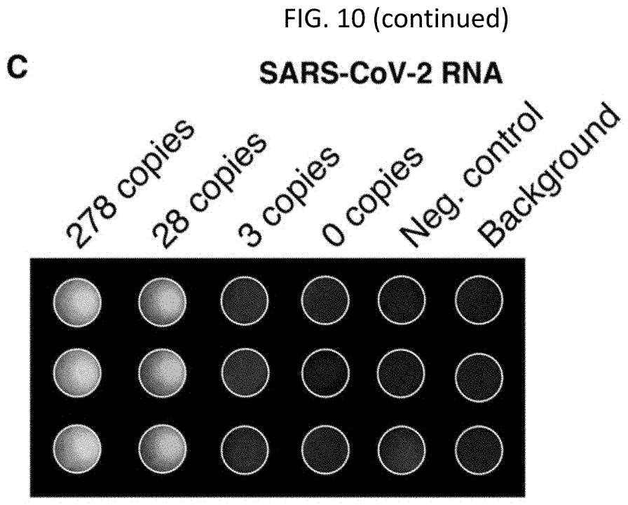

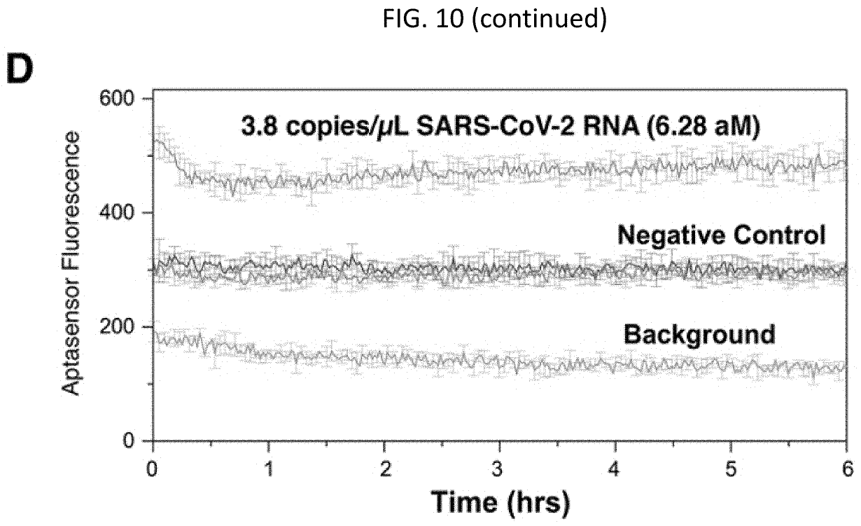

The top-performing aptasensors were next coupled to isothermal amplification reactions to ensure that they could reach the clinically relevant detection limit of SARS-CoV-2 RNA. A custom primer design algorithm was implemented to select NASBA and RPA primers having optimal specificity, secondary structure, and sequence composition to enable amplification of the region about the binding site of the aptasensors. Experiments were first conducted to screen the resulting primers in 6-4, NASBA reactions supplied with 185 copies of SARS-CoV-2 RNA obtained from cultured virions. Reactions were incubated at 41° C. using 10 different NASBA primer pairs designed to amplify the Orflb target region (see Table 8 for primer sequences). The resulting amplicons were then added to solutions containing the Orflb Broccoli aptasensor and DFHBI-1T. FIG. 10A shows the time-course measurements of aptasensor fluorescence for all 10 primer combinations along with the fluorescence from a negative control NASBA reaction lacking the viral RNA template and the background signal from a solution containing the DFHBI-1T fluorogen without the aptasensor present. These measurements conducted at 37° C. reveal a substantial range of different fluorescence outputs from the primers with the pair NASBA_broc_rot_arb_b08_covid19_ORFlb_A_0003_fwd/NASBA _broc_rot_arb_b08_covid19_ORFlb_A_0003_rev providing the strongest signal.

The optimal primer pair was then combined with the Orflb Broccoli aptasensor for a series of experiments supplying the amplification reactions with different concentrations of cultured SARS-CoV-2 RNA. These experiments revealed that the Broccoli aptasensor could provide significant fluorescence output for sample concentrations down to 23 RNA copies/μL, which corresponds to only 28 copies of RNA supplied to the 6-μL NASBA reaction. In addition, significant fluorescence was observed from the aptasensors immediately during the 37° C. measurement in the plate reader (FIG. 10B). FIG. 10C shows a photograph of the reactions from these experiments demonstrating the strong green fluorescence that can be detected upon activation of the Broccoli aptasensors with as little as 28 copies of viral RNA. Additional assay detection limit tests have shown that the test can detect SARS-CoV-2 RNA down to concentrations of 3.8 copies/μL (6.28 aM) in the NASBA reaction. FIG. 10D shows the aptasensor fluorescence from these assays compared to a negative control, lacking any SARS-CoV-2 RNA in the NASBA reaction, and the background signal from the DFHBI-1T. A statistically significant difference in all three conditions is observed confirming a detection limit of 6.28 aM.

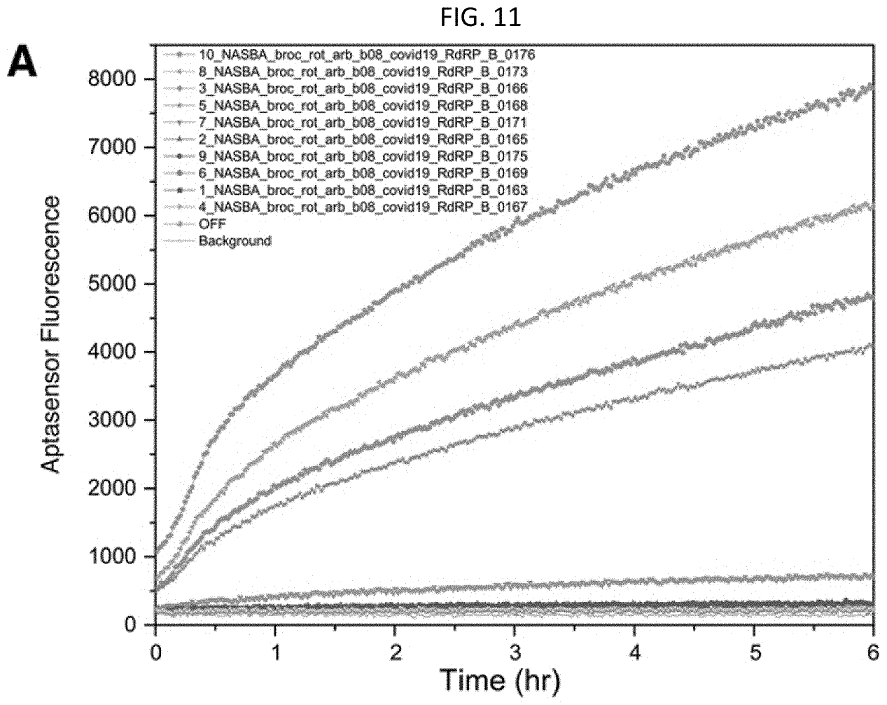

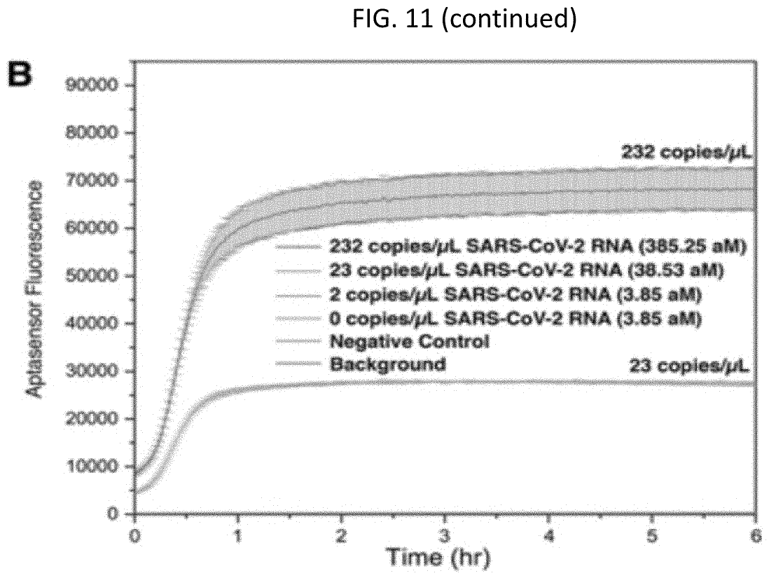

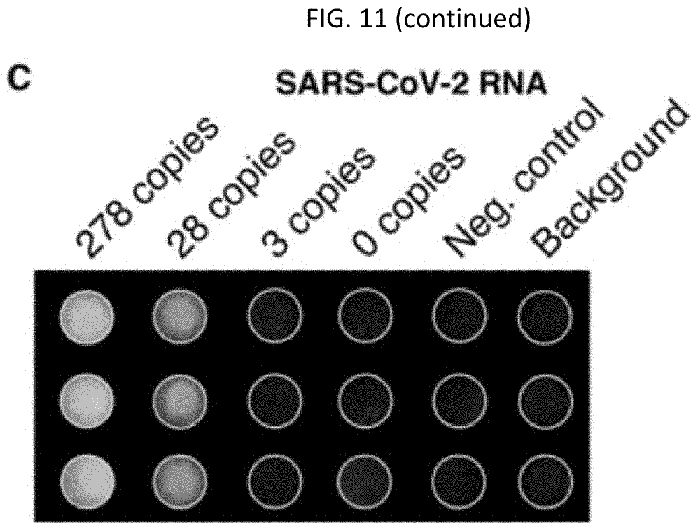

To demonstrate the robustness of the approach, a second set of NASBA primers was designed for the RdRP gene target of SARS-CoV-2 (see Table 9 for primer sequences). Primer screening experiments in FIG. 11A show an optimal primer pair (NASBA_broc_rot_arb_b08_covid19_RdRP_B_0176_fwd/NASBA broc_rot_arb_b08_covid19_RdRP_B_0165_rev) providing the strongest fluorescence from the corresponding RdRP Broccoli aptasensor. These primers were then used in a limit of detection test with the aptasensor (FIG. 11B). These experiments demonstrated that the assay could again detect down to 23 copies/μL of the virus, corresponding to 28 copies of RNA supplied to a 6-μL NASBA reaction. Using this aptasensor, fluorescence was again detectable immediately in the plate reader (FIG. 11B) with strong output within 30 minutes at 37° C. Photographs of these reactions show strong visible fluorescence from reactions with 278 and 28 copies of the virus supplied to the NASBA reaction (FIG. 11C). Experiments also have been performed to test the ability of the SARS-CoV-2 aptasensors to detect the amplicons generated by reverse transcription loop-mediated amplification (RT-LAMP). In principle, aptasensors can be used to detect single-stranded DNA produced from RT-RPA with asymmetric (i.e., unequal) primer loadings or single-stranded loop regions of RT-LAMP products. Alternatively, both RT-RPA and RT-LAMP products can be transcribed to provide an RNA product that can be detected with the aptasensors.

### Aptasensors for Detection of Reverse Transcription Recombinase Polymerase Amplification (RT-RPA) Amplicons

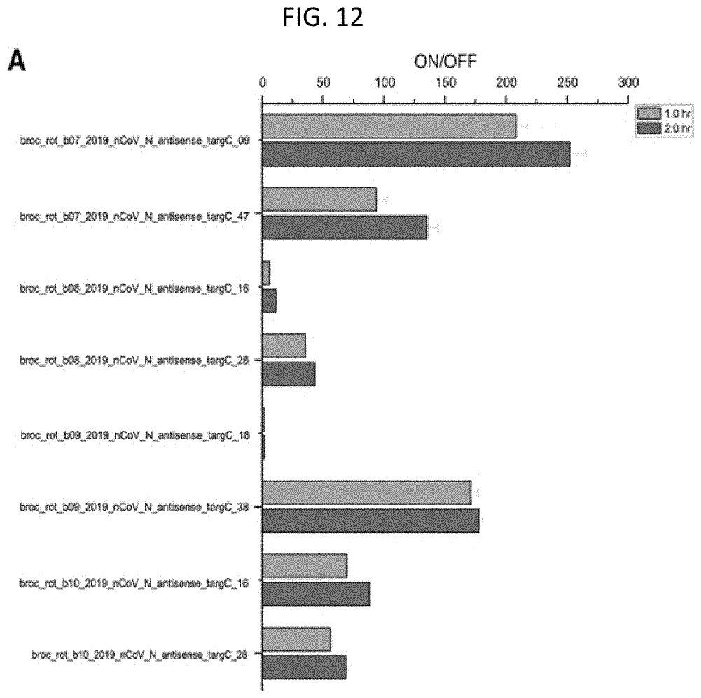

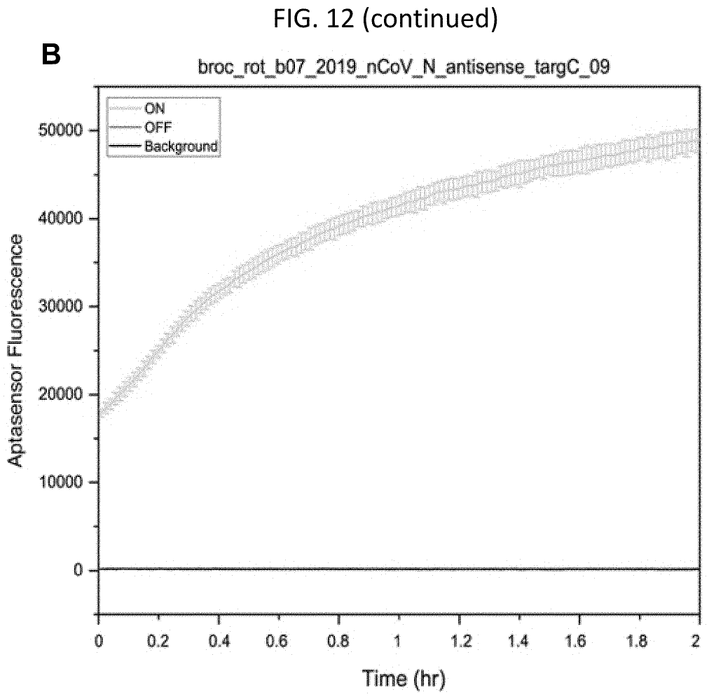

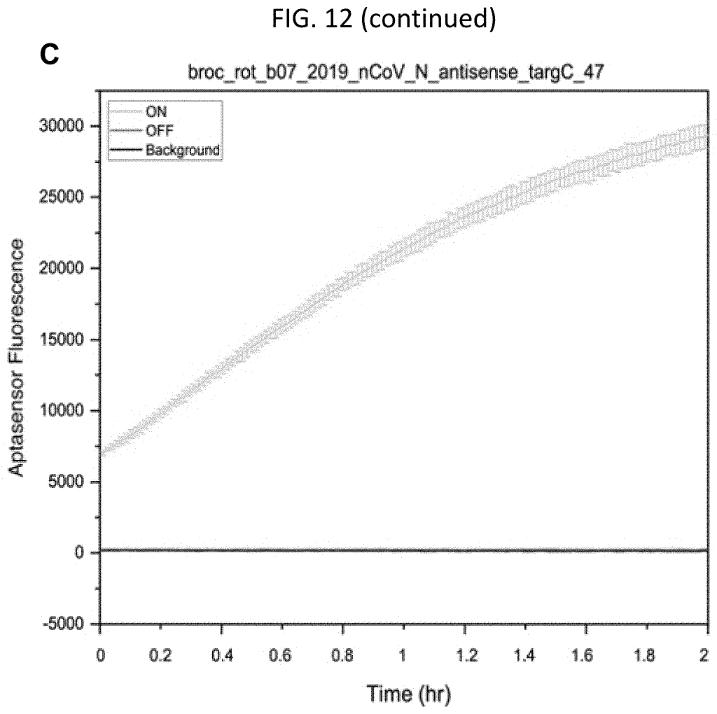

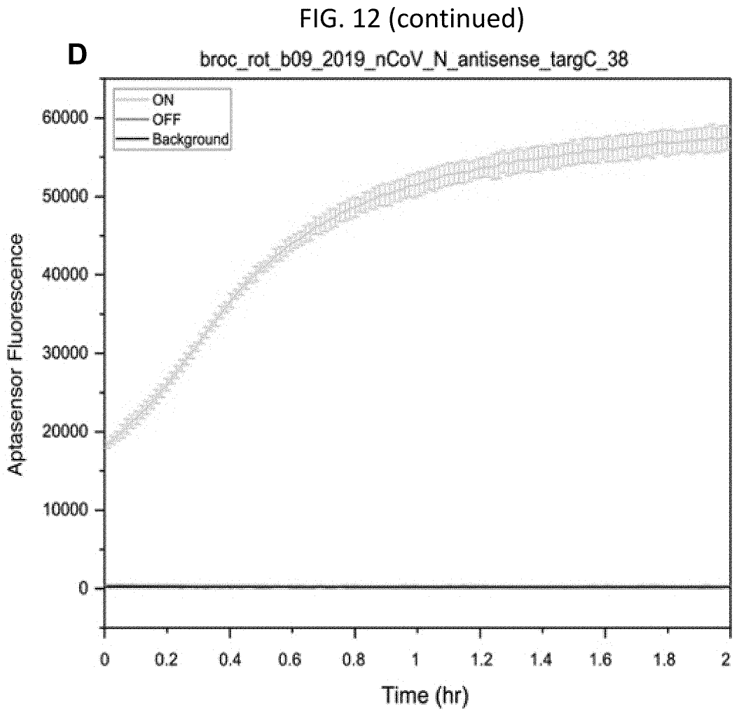

In preparation for experiments using RT-RPA for amplification, we have also validated a library of aptasensors targeting an RT-RPA amplicon. This particular amplicon is generated through an RT-RPA reaction amplifying the antisense orientation of the N gene of SARS-CoV-2 and it appends a T7 promoter site to the viral sequence to enable subsequent in vitro transcription. In previous experiments using toehold switches for sequence verification, the RT-RPA primers have provided a detection limit of 0.5 aM, corresponding to 15 copies of SARS-CoV-2 RNA in the 50 μL RT-RPA reaction. FIG. 12 shows performance data from the library of Broccoli aptasensors targeting the transcript generated from the amplicon (SEQ ID NOs:111-118; see Table 10). Three of the eight sensors provide ON/OFF ratios above 100-fold after two-hour reactions and the highest performance aptasensor provides an impressive 253-fold signal increase with the target (FIG. 12A). Time-course curves from the aptasensors reveal very fast activation speeds in plate reader measurements at 37° C. and very low levels of fluorescence leakage in the OFF state when the target RNA is absent (FIG. 12B-12D).

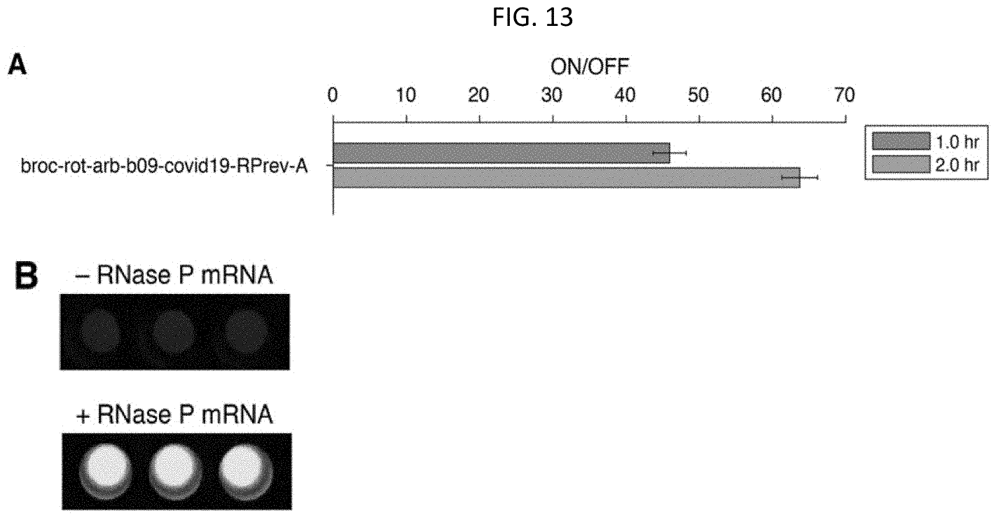

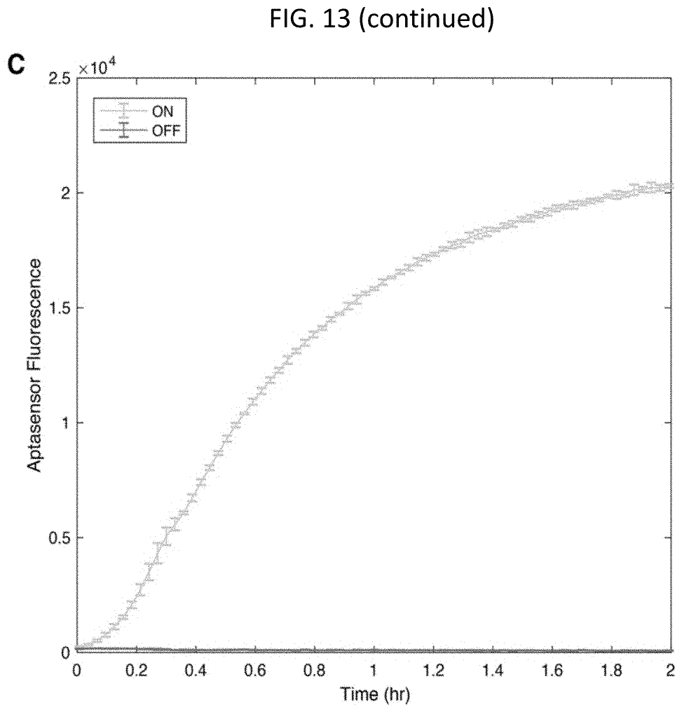

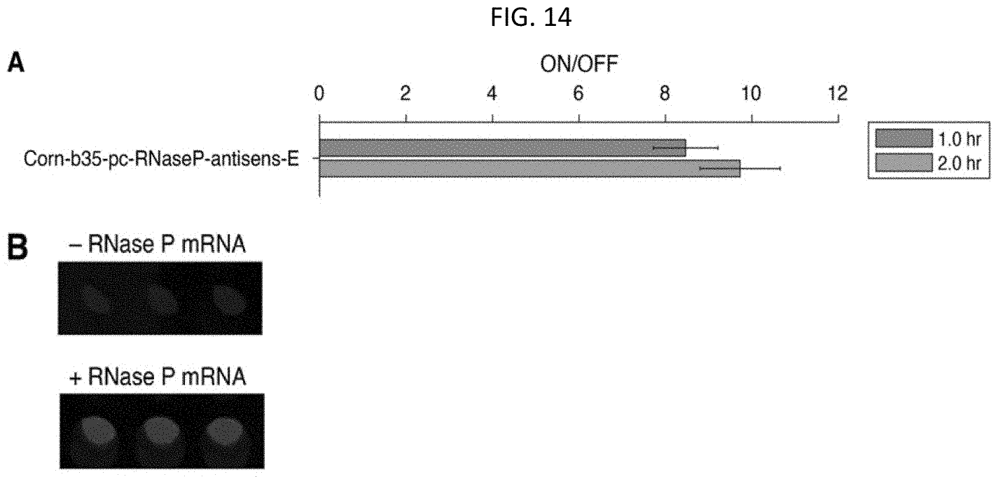

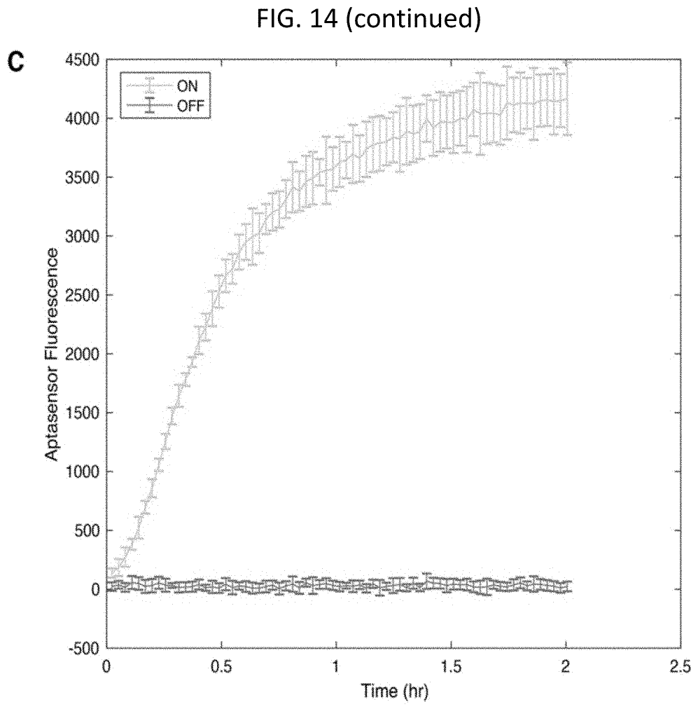

Parallel sample control reactions that detect nucleic acids expected to be present in all samples are valuable for ensuring proper sample processing during tests. Aptasensors for sample controls were implemented to detect the human RNase P mRNA (SEQ ID NOs:119-120; see Table 11). FIG. 13 shows validation data from a Broccoli aptasensor targeting the antisense orientation of RNase P. The aptasensor provides an ON/OFF ratio greater than 45-fold in response to RNase P and provides clearly visible green fluorescence. Significant fluorescence is obtained from the Broccoli aptasensor within 15 minutes. A Corn aptasensor for the same RNase P target was also developed. This sensor provided at least an 8-fold ON/OFF ratio (FIG. 14A) and discernible yellow fluorescence by eye (FIG. 14B). This Corn aptasensor also activated strongly within 15 minutes of exposure to the target RNA (FIG. 14C).

### Aptasensors for Detection of Reverse Transcription Loop-Mediated Isothermal Amplification (RT-LAMP) Amplicons

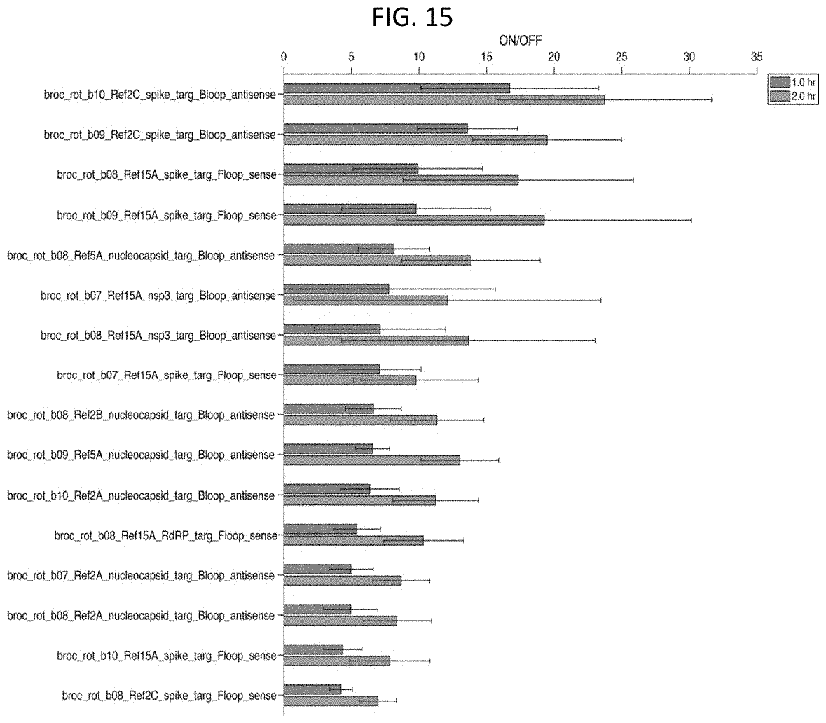

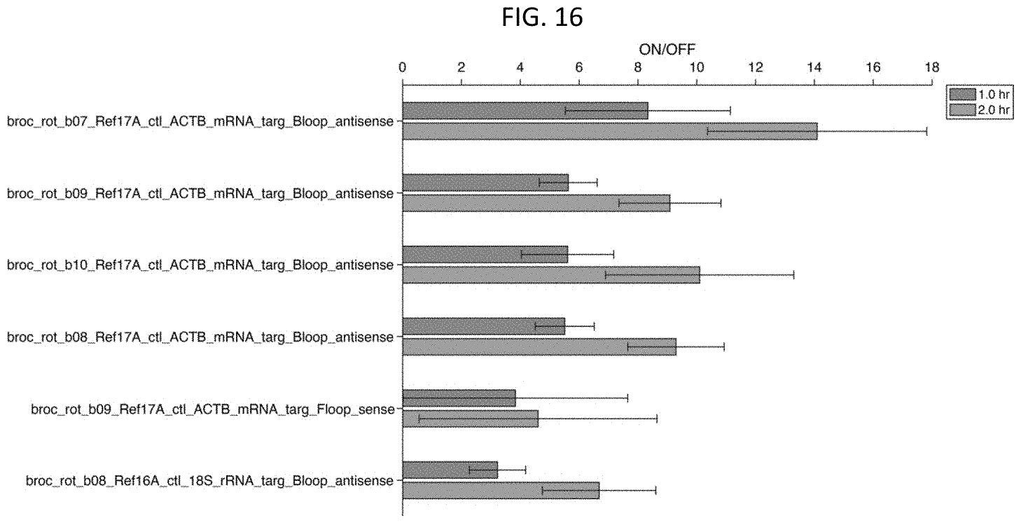

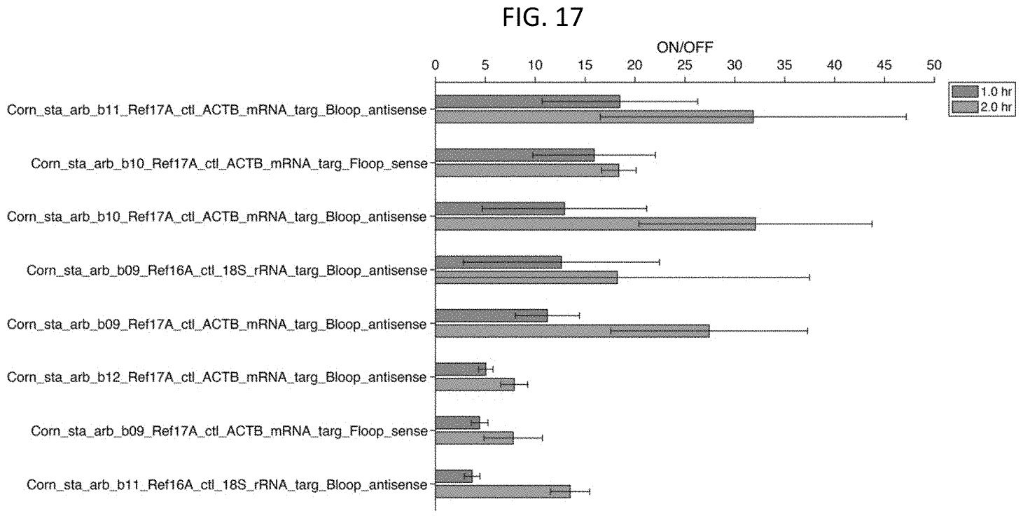

To reduce the likelihood of false-positive RT-LAMP assays, we also developed a library of aptasensors for detection of the DNA products of RT-LAMP amplification assays. It is noted that the LAMP primer sequences provided herein for FIGS. 15-17 were taken from published literature, but the aptasensors developed herein have not been reported before. Aptasensors based on Broccoli and Corn with designed to target the single-stranded loop regions of LAMP reaction DNA products. These aptasensors were tested in reactions by adding long stem-loop DNA strands representative of the expected LAMP products. FIG. 15 shows the ON/OFF fluorescence ratios for 20 Broccoli aptasensors for different genes from SARS-CoV-2 (SEQ ID NOs:121-136; see Table 12). The sensors provide up to 24-fold increases in fluorescence in response to target DNAs representative of the RT-LAMP products. Aptasensors were also tested against the sample control mRNA actin beta (ACTB) and the 18S ribosomal RNA (rRNA). Broccoli aptasensors provided up to 14-fold ON/OFF ratios (FIG. 16) (SEQ ID NOs:137-142; see Table 13), while Corn aptasensors yielded up to 32-fold ON/OFF (FIG. 17) (SEQ ID NOs:143-150; see Table 14). These results indicate that the aptasensors can be added to LAMP and RT-LAMP reactions to provide a critical sequence verification step for detection of SARS-CoV-2 and other pathogens.

### Detection Equipment

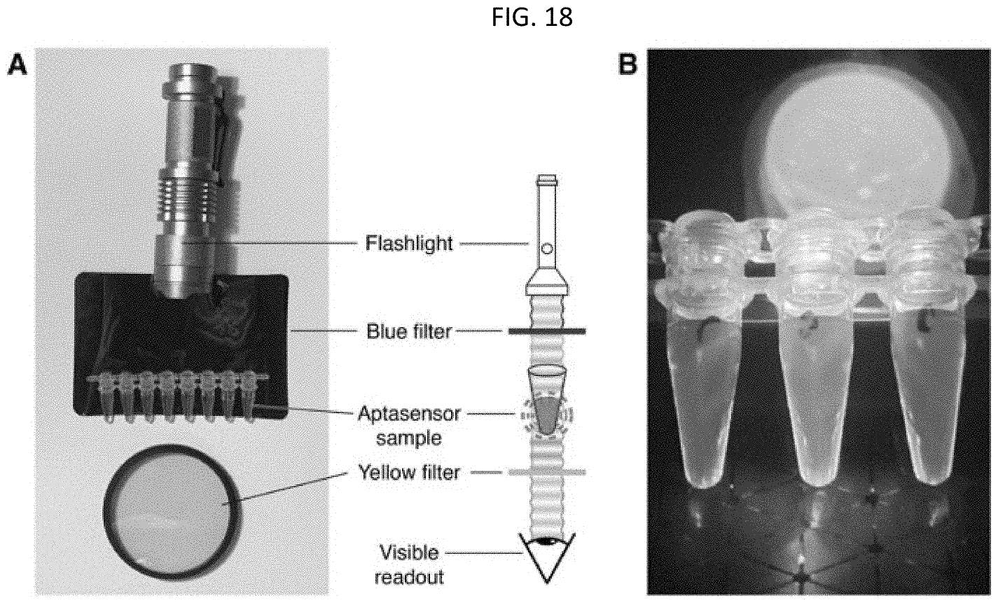

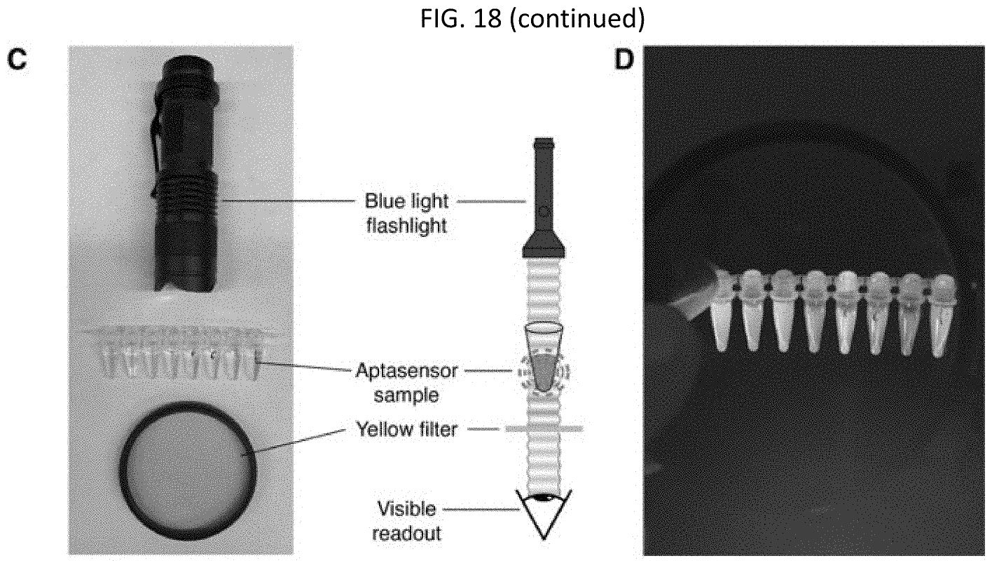

The capacity of these diagnostic tests to detect SARS-CoV-2 RNA using isothermal reactions close to human body temperature suggests that these systems could be used for in-home assays. Accordingly, the use of readily available components to detect fluorescence from activated Broccoli aptamers was explored. FIG. 18A shows one sample test kit for fluorescence measurement. The test kit consists of an LED Flashlight (7W 300 LM Mini LED, Wayllshine: $6.99), a blue filter (Pieces Universal Gels Lighting Filter Kit, Selens: $0.60/filter), and a yellow filter (Tiffen 58 mm 12 Filter (Yellow): $14.82). By transmitting white light from the mini LED flashlight through the blue filter, the aptasensor is excited by blue light. This input light is then filtered out using the yellow optical filter leaving the green fluorescence from the Broccoli aptamer to be distinguished by eye or smartphone camera. Green fluorescence from the Broccoli aptamer solutions can be clearly seen in FIG. 18B. To simplify the detection setup further, we also replaced the flashlight/blue filter combination with a blue light flashlight (Blue LED 3 Mode Flashlight, Wayllshine: $8.99), while retaining the yellow filter ($14.82) as shown in FIG. 18C. With this simplified configuration, Broccoli fluorescence could more easily be detected (FIG. 18D). Experiments were also performed in which the yellow filter was replaced with widely available yellow goggles used for filtering out UV light (Calabria 1003 Large Fit-Over UV Protection in Yellow, $12.95). These goggles also enabled the Broccoli fluorescence to be readily seen by eye. These studies demonstrate that results from the aptasensor assays can be detected with $20 to $24 of equipment. It is expected that such costs can be decreased further with the use of less expensive light sources and yellow filters and through larger-volume purchasing. The reagent costs for detection of a single transcript using 5-4, NASBA reactions come to $3.09, with 86% of the cost arising from the NASBA components. In addition, it is expected that the reactions should be capable of being lyophilized for room-temperature distribution and storage, since both the NASBA and T7 RNA polymerase used for sensor synthesis have previously been shown to be stable under freeze drying.

### Conclusions

As demonstrated herein, a simple assay has been developed that provides specific detection of SARS-CoV-2 RNA down to sample concentrations of 23 RNA copies/μL. In this assay, a target RNA is amplified using an isothermal amplification method, such as NASBA, RT-RPA, or RT-LAMP. Then, to reduce the possibility of false positive results due to non-specific amplification, computer-designed aptasensors are employed to verify the sequence of the amplified products and produce a strong and readily visible fluorescence signal for a positive test. Importantly, the reactions in this assay can be accomplished using simple heating procedures and incubation near human body temperature, facilitating the transition to in-home use. Furthermore, the assays can be visualized using simple, readily available equipment that can be obtained for $20 to $24. Each target RNA can be detected in the reactions for as little as $3.09 per result. This assay can be used to detect more than one analyte at the same time by harnessing the different optical properties of various aptamer/fluorogen combinations. This capability can be used to reduce assays costs and reduce the likelihood of false positive results. In addition to Broccoli and Corn, other aptamers (e.g., Red and Orange Broccoli) can be used in the general toehold-mediated aptasensor design described herein. Inclusion of additional aptamers could be used to increase assay multiplexing capacity or allow the assay results to be interpreted by other fluorescence detection systems.

### Example 2

In the following Example, the inventors describe an improved aptasensor design and an improved SARS-CoV-2 detection assay that employs reverse transcription loop-mediated amplification (RT-LAMP) for isothermal amplification. Further, they describe multiplexed and one-pot variations of the RT-LAMP-based assay, and they validate their method against clinical samples.

### Development of an Improved Aptasensor Design

The improved aptasensor design makes a few key changes over the aptasensors described in Example 1. This design features an improved sensing hairpin. The previous aptasensors employed a hairpin that spanned 20 nucleotides and featured two bulges, 4 and 8 bases from the top of the stem, to reduce the likelihood of premature transcriptional termination. The improved aptasensors do not have any bulges within the stem, which reduces signal leakage without decreasing transcriptional efficiency. The stem itself is varied from 12 bp to 21 bp, depending on the properties of the target RNA and the particular output aptamer. For output aptamers that have a middle stem-loop that does not have a fully conserved sequence (e.g. Broccoli, Red Broccoli, and Orange Broccoli), we designed appropriate RNA sequences for each aptasensor that will ensure that this middle stem-loop folds into a strong secondary structure and avoids pairing elsewhere within the aptasensor (see cyan region of FIG. 19). Use of this highly stable middle stem-loop (or inner clamp), typically programmed to have an 8-bp stem and 4-nt loop, helps encourage formation of the aptamer once the sensor is activated, thus leading to a stronger ON-state signal.

In addition to these changes, we also tested the updated fluorogens BI [full name: (Z)-3-((1H-benzo[d]imadazol-4-yl)methyl)-5-(3,5-difluoro-4-hydroxybenzylidene)-2-methyl-3,5-dihydro-4H-imidazol-4-one] and OBI [full name: 3,5-difluoro-4-hydroxybenzylidene-imidazolinone-2-oxime-1-benzoimidazole (OBI)]. These new fluorogens provided stronger fluorescence when bound to the aptamers, with BI pairing with Broccoli aptasensors and OBI pairing with Red Broccoli aptasensors.

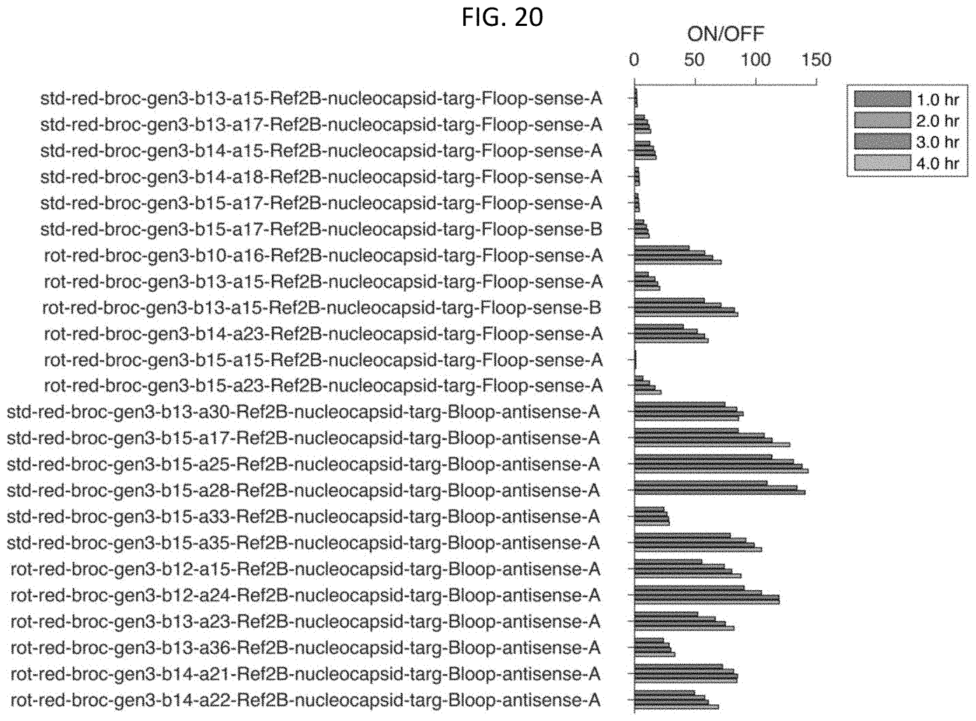

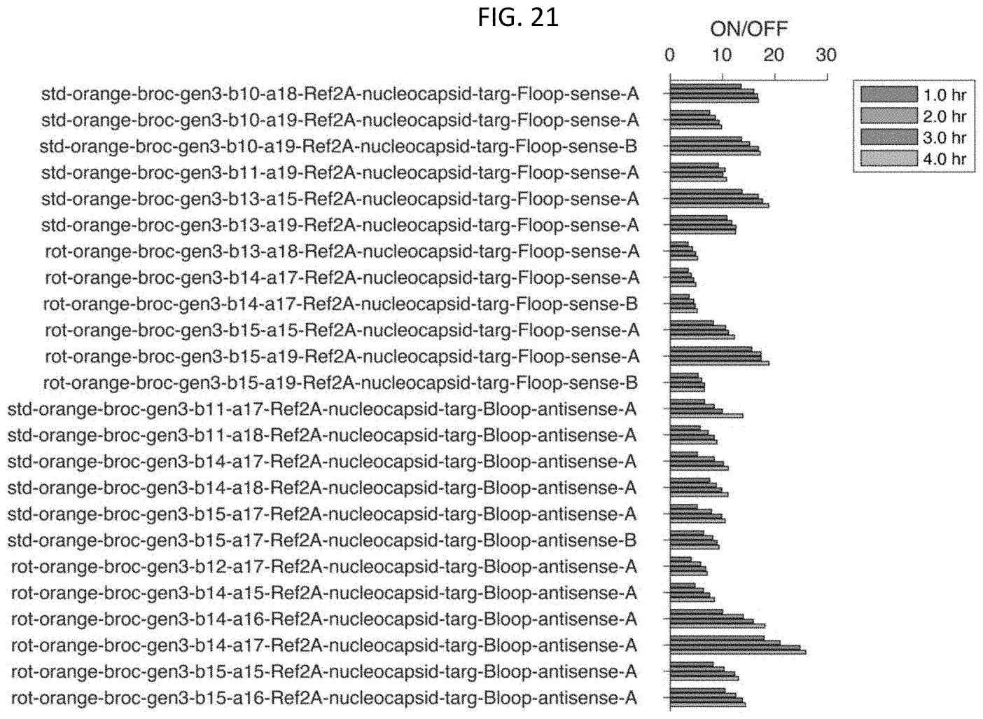

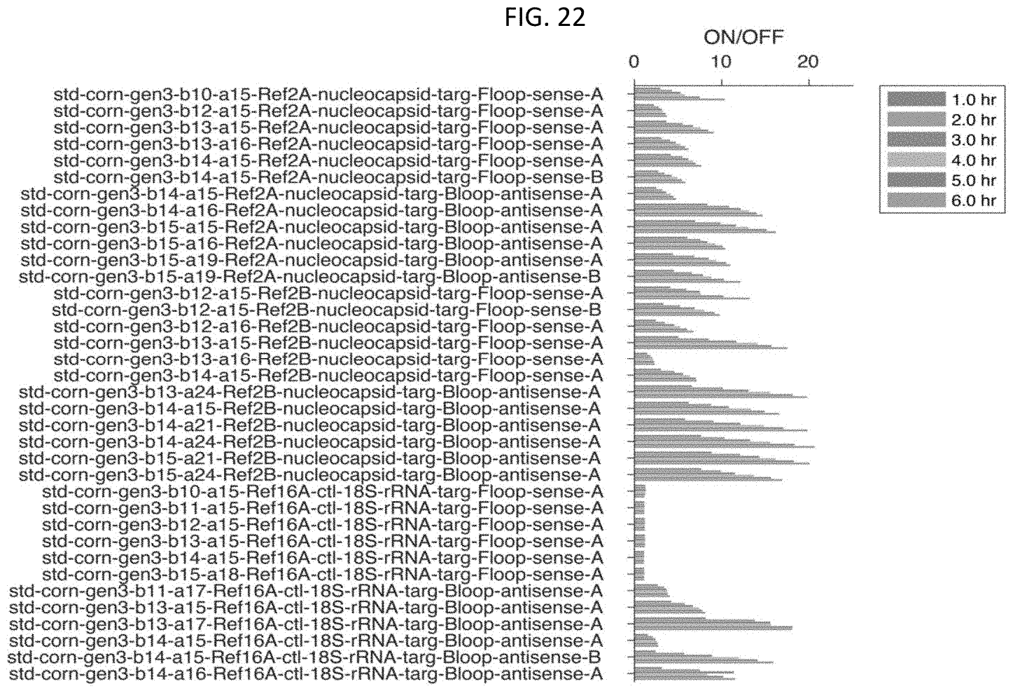

FIGS. 20-22 show representative data from improved aptasensors with outputs Red Broccoli, Orange Broccoli, and Corn, respectively. When the Red Broccoli system is paired with the fluorogen OBI, impressive signal increases of over 100-fold can be obtained in the presence of the target nucleic acid from SARS-CoV-2 (FIG. 20) (SEQ ID NOs:151-174; see Table 15). In addition, both standard and rotated versions of the output aptamer are functional, wherein the rotated version is a circular permutation of the original standard aptamer. The Orange Broccoli system paired with DFHO dye can provide ON/OFF ratios exceeding 20-fold against SARS-CoV-2 for both standard and rotated versions of the output aptamer (FIG. 21) (SEQ ID NOs:175-198; see Table 16). Tests with improved Corn aptasensors in DFHO provided signal increases above 15-fold for SARS-CoV-2 targets and the sample control 18S rRNA (FIG. 22) (SEQ ID NOs:199-246; see Table 22). For the Corn system, since it contains a conserved middle stem loop, an inner clamp was not used, but these systems did use the updated stem structure.

### RT-LAMP/Aptasensor Detection Scheme

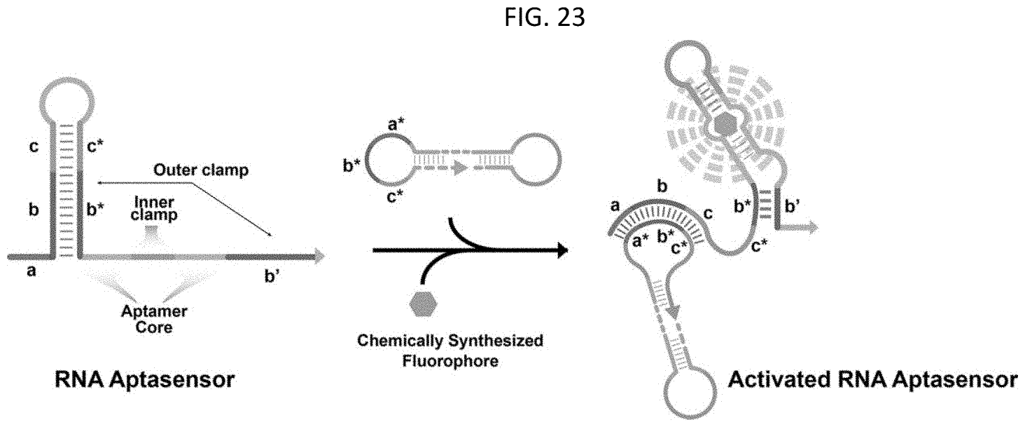

We have developed an improved SARS-CoV-2 detection assay employing reverse transcription loop-mediated amplification (RT-LAMP) for isothermal amplification of the SARS-CoV-2 genome and the sample control RNA (either ACTB mRNA or 18S rRNA). RT-LAMP has an operating temperature of —60° C. to 65° C. and uses a set of four to six primers to generate DNA products containing exposed, single-stranded loop domains. These loop domains are targeted by two to four of the LAMP primers and are essential for the amplification process. We thus designed aptasensors that targeted these exposed loop domains, as illustrated in FIG. 23, to activate aptamer fluorescence.

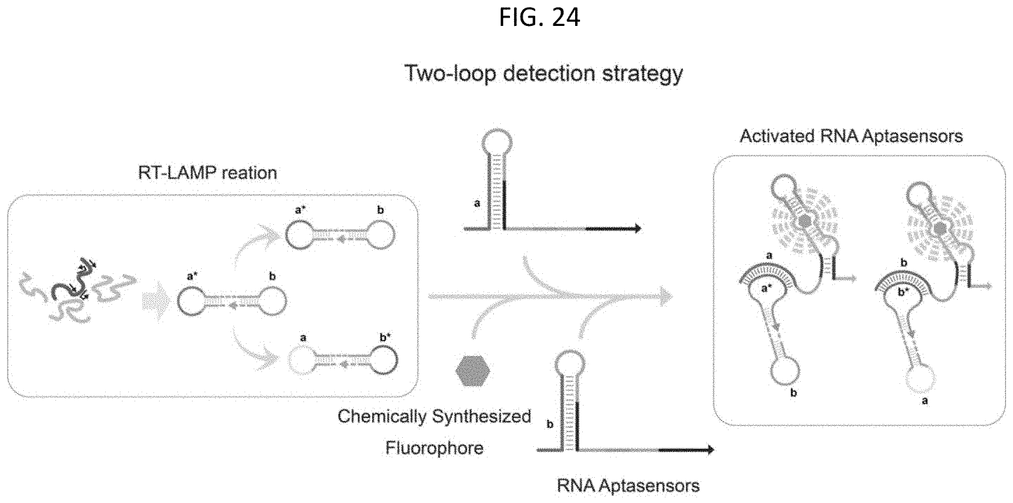

Since RT-LAMP produces amplicons with two loop regions that have unrelated sequences, we also implemented a scheme to target the two independent loop regions in the same reaction (FIG. 24). This approach effectively doubles the concentration of targets available for detection and thus increases signal output and/or decreases reaction time.

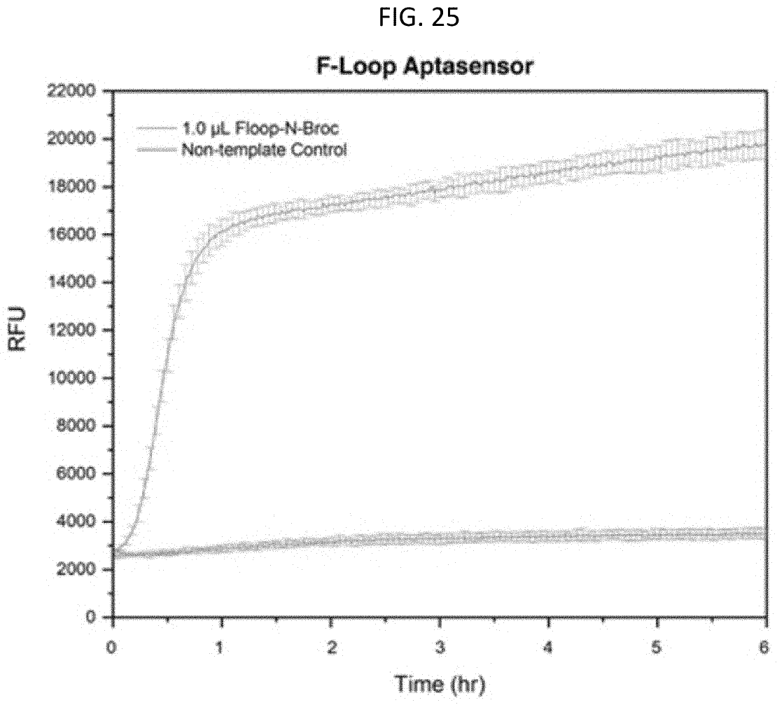

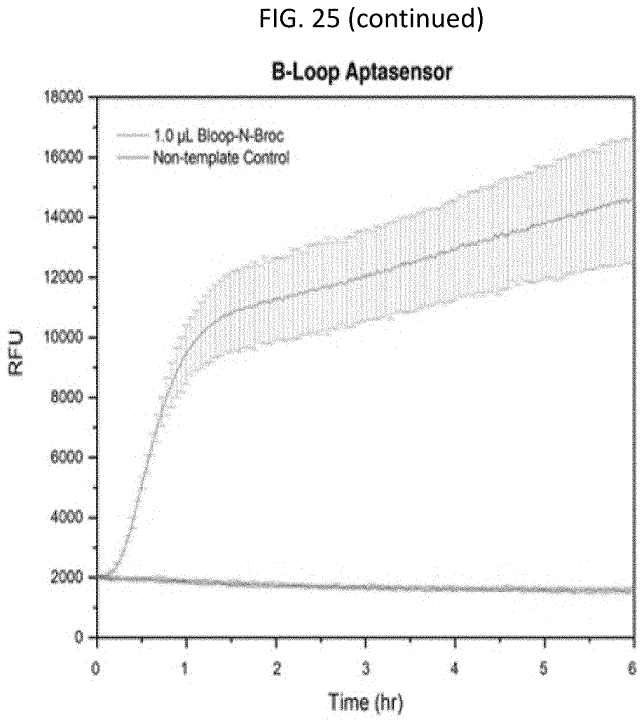

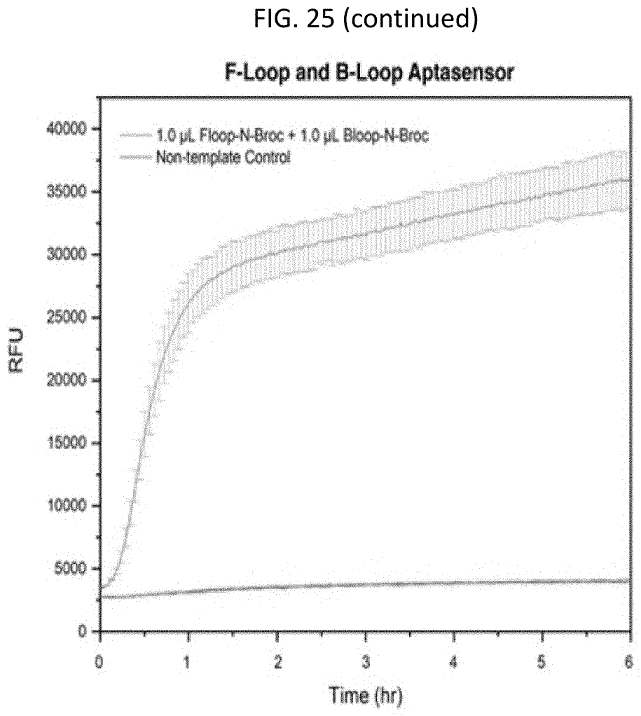

Validation data from of the dual loop detection scheme is presented in FIG. 25 (SEQ ID NOs:247-248; see Table 18). On their own, aptasensors for the F-Loop and B-Loop of the RT-LAMP DNA amplicon produce a fluorescence signal of 10,000 to 16,000 relative fluorescence units (RFU) within a one-hour reaction period. In comparison, the dual loop system using aptasensors targeting both the F-Loop and B-Loop simultaneously provides roughly the sum of the two independent signals at ˜26,000 after one hour. The increase in signal leads to a concomitant decrease in time-to-result of ˜35%.

We proceeded to evaluate a combined two-pot assay wherein RT-LAMP is performed first on a sample of RNA for 10 minutes to 60 minutes at 60° C. to 65° C. The resulting RT-LAMP product is then diluted into a second pot or reaction vessel containing the aptasensors designed to target the loop domains of the RT-LAMP amplicons. Detection limit tests were performed with the two-pot RT-LAMP aptasensor assay using different combinations of primers and different aptasensors to gauge its sensitivity. In general, the sensitivity of the assay is equivalent to RT-qPCR tests conventionally used for SARS-CoV-2 detection in clinical samples.

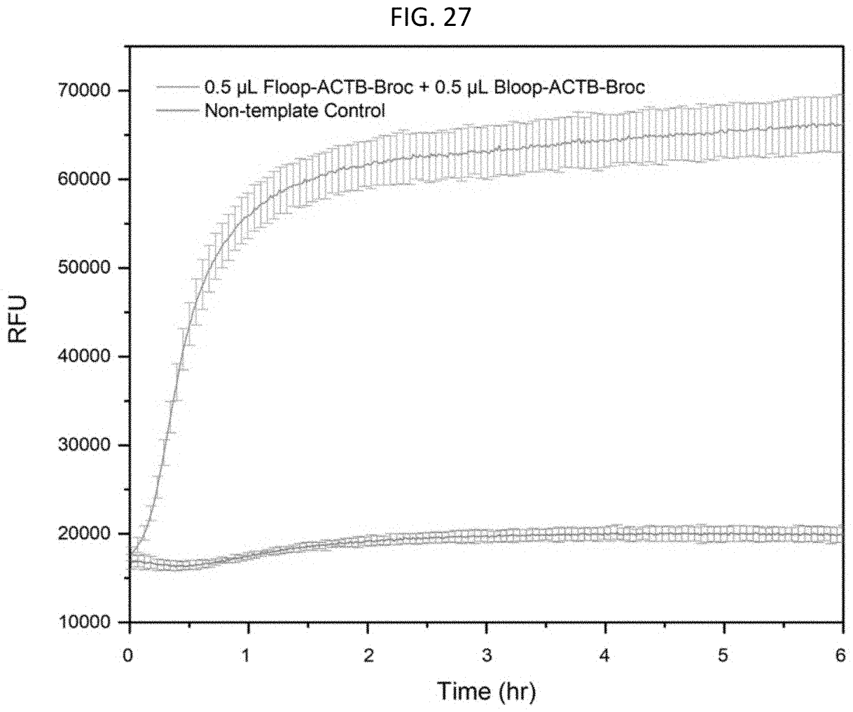

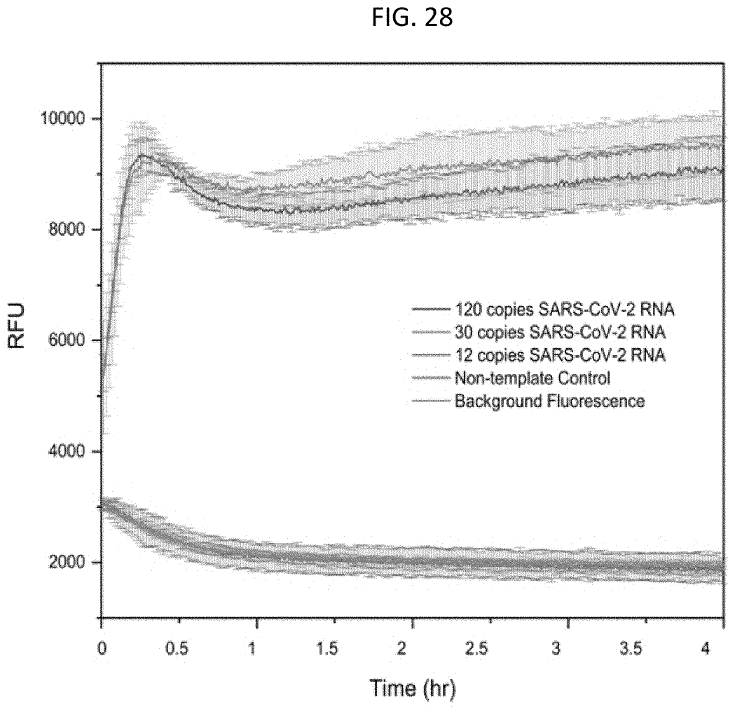

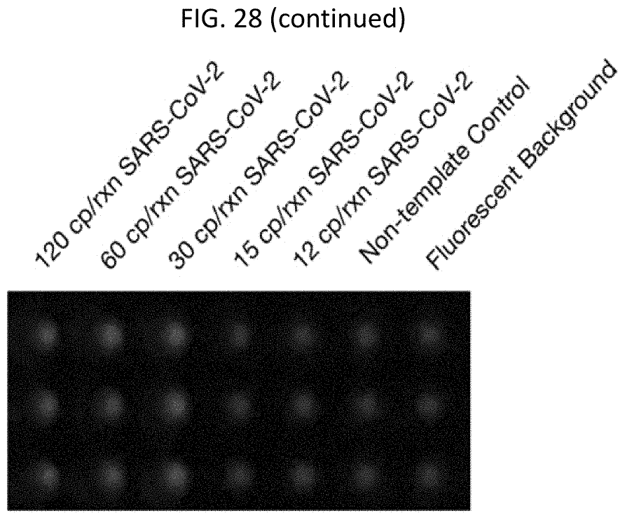

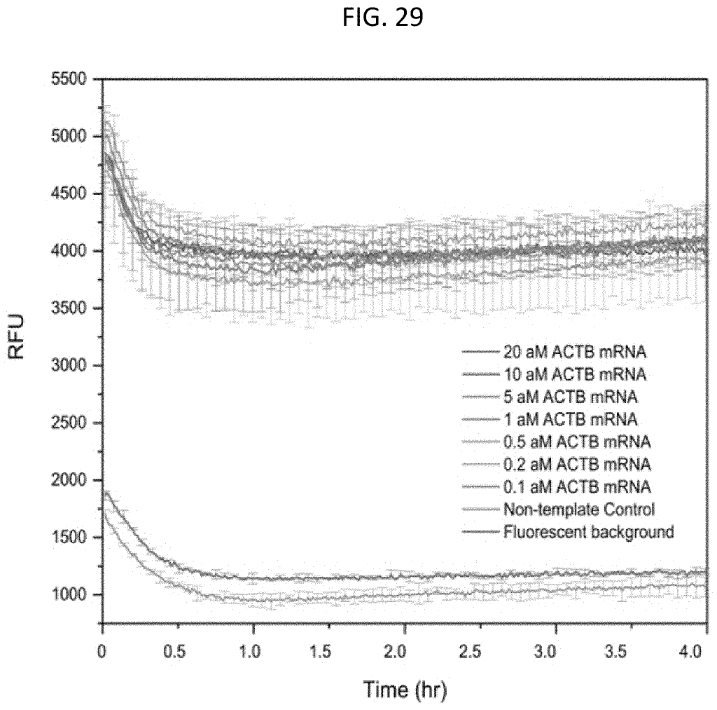

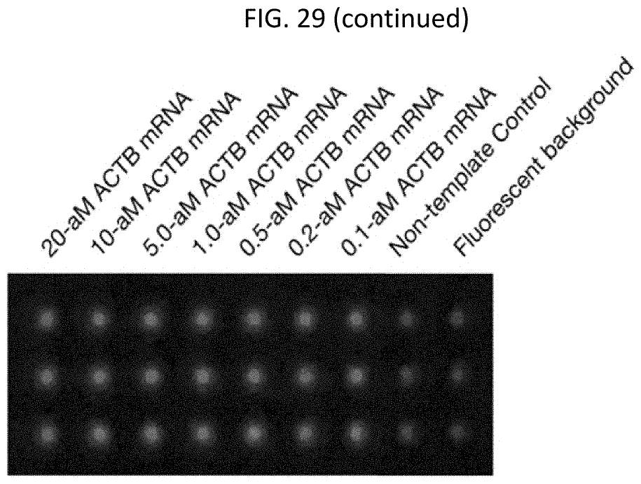

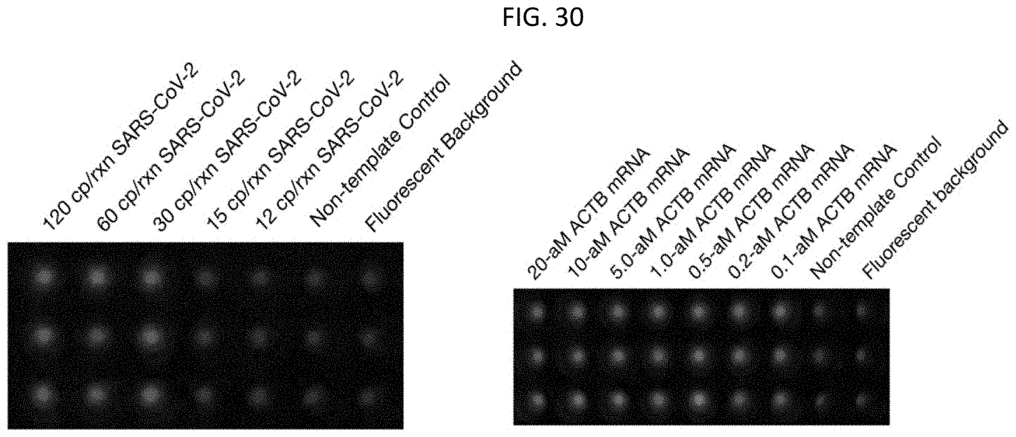

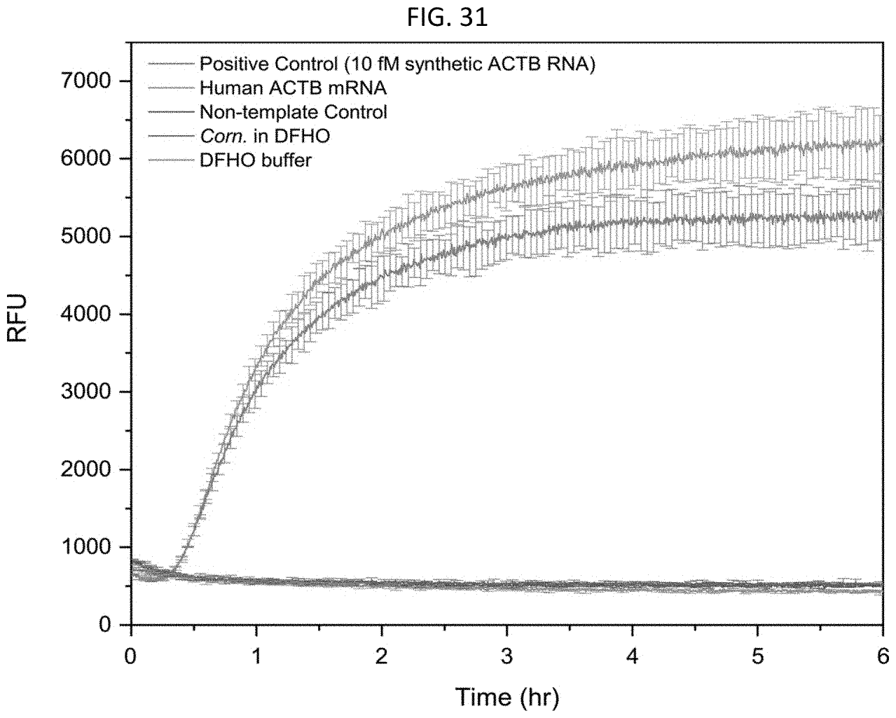

FIGS. 26-31 show results from these experiments. Using RT-LAMP primers amplifying the spike gene of SARS-CoV-2 and a single Broccoli aptasensor, we achieved a limit of detection of 2 copies of SARS-CoV-2 per 30-4, reaction or 0.13 aM (FIG. 26) (SEQ ID NO:121; see Table 19). A pair of Broccoli aptasensors targeting two RT-LAMP loops from amplification of the ACTB mRNA control provided a limit of detection of 3.6 copies per 30-μL reaction or 0.2 aM (FIG. 27) (SEQ ID NOs:249-250; see Table 20). Experiments performed with the additional aptamer output Red Broccoli provided a visible red/orange color that could be seen by eye using a blue light source and an orange optical filter (FIGS. 28 and 29) (SEQ ID NOs:166 and 251-252; see Tables 21 and 22). Detection limits of 30 copies of SARS-CoV-2 (2 aM) were obtained coupling the Red Broccoli aptasensors to RT-LAMP and of 0.1 aM for the ACTB mRNA with RT-LAMP. Orange Broccoli aptasensors provided a yellow/green fluorescence and detection limits of 30 copies per reaction (2 aM) and 0.1 aM for the SARS-CoV-2 RNA and ACTB mRNA, respectively, following RT-LAMP (FIG. 30) (SEQ ID NOs:253-256; see Table 23). Corn aptasensors targeting the RT-LAMP loops from amplification of the ACTB mRNA control provided a limit of detection of 3.6 copies per 30-4, reaction or 0.2 aM (FIG. 31)(SEQ ID NO:143; see Table 31).

### Two-Channel, Two-Pot RT-LAMP/Aptasensor Assay

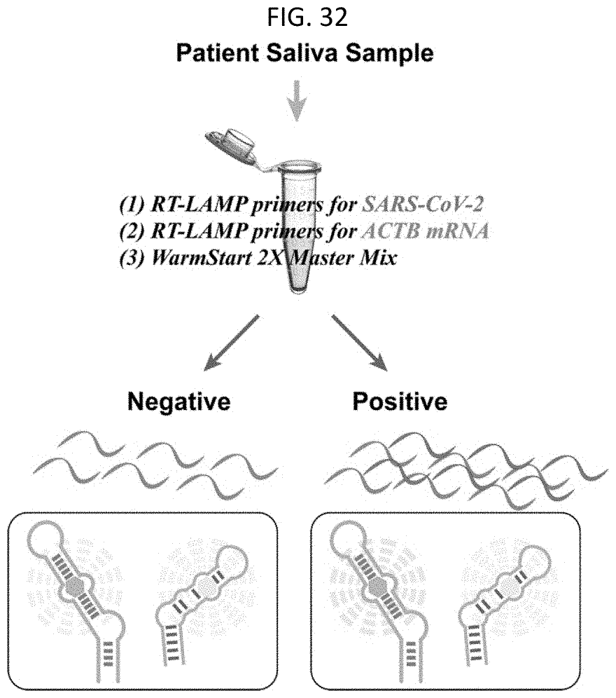

A limitation of the two-pot RT-LAMP assays of the previous section is that they only detected one target amplicon at a time. Taking advantage of the different spectral properties of aptamers with their companion fluorogens, we implemented a two-channel two-pot RT-LAMP assay capable of simultaneously detecting two different targets. Such as assays can lead to reduced costs and processing time. The two-channel reaction employs a first-step RT-LAMP reaction where the primers for both targets are present. These targets are then amplified over the course RT-LAMP for 10 to 60 minutes at 60° C. to 65° C. The reaction products are then diluted into a second pot containing aptasensors for each target and their companion fluorogens. FIG. 32 illustrates the general procedure for simultaneous detection of SARS-CoV-2 along with the sample control ACTB mRNA. For all clinical samples, the ACTB mRNA should be present and cause production of a yellow fluorescence signal from a Corn aptasensor. In clinical samples that are positive for SARS-CoV-2, the presence of viral RNA will elicit a green fluorescence signal from the cognate Broccoli aptasensor. Absence of any fluorescence signal from the reaction is evidence of a problem and indicates that the test should be rerun.

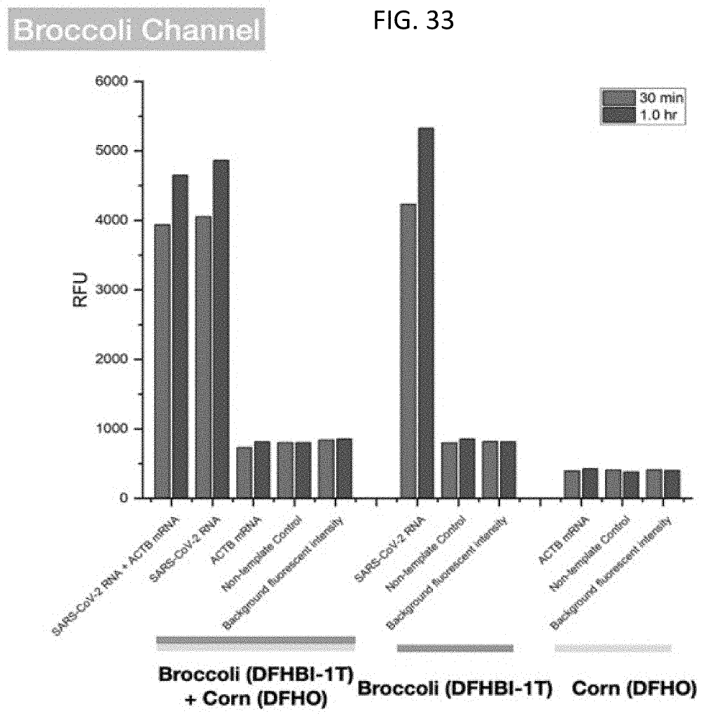

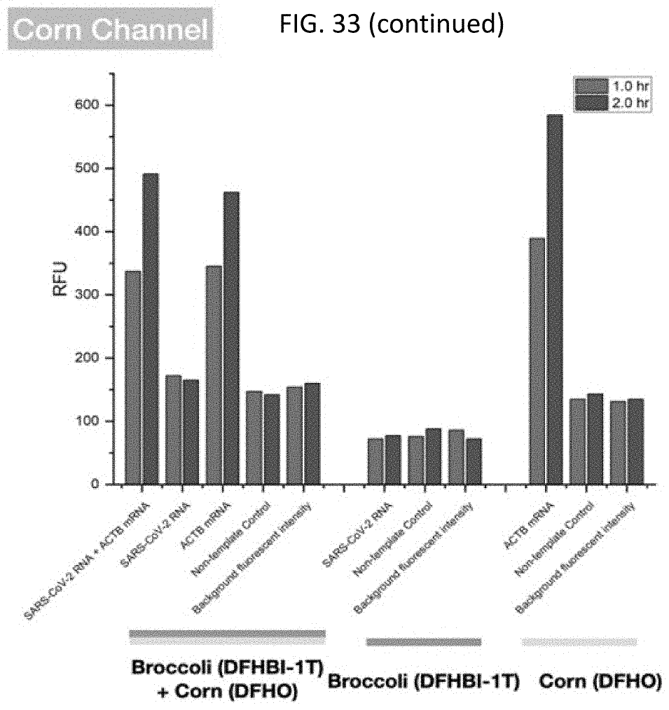

The two-channel, two-pot reaction was tested with input concentrations of 0.478 aM of SARS-CoV-2 RNA and 150 aM of ACTB mRNA. RT-LAMP was performed in a single reaction with 12 total primers for amplification of both SARS-CoV-2 and ACTB RNAs simultaneously. The resulting products were then added to detection reactions containing different combinations of aptasensors and fluorogens. Fluorescence output from each of the reactions was measured in the Broccoli channel with green fluorescence and the Corn channel with orange fluorescence (FIG. 33) (SEQ ID NOs:121 and 143; see Table 25). Samples containing both the Broccoli and Corn aptasensors showed the expected strong Broccoli and Corn fluorescence in cases where the SARS-CoV-2 or ACTB mRNA were presented, respectively. Reactions containing only one of the dye molecules also displayed the expected results with strong green fluorescence only observed with the Broccoli/DFHBI-1T present along with SARS-CoV-2 RNA. Similarly, strong orange fluorescence was only observed with the Corn/DFHO system when ACTB mRNA was added. Based on these results, we applied the two-channel, two-pot reaction for detection of SARS-CoV-2 in clinical samples. See the final section (“Validation of assay against clinical samples”) for a description of the results with the clinical samples.

### One-Pot RT-LAMP/Aptasensor Assays

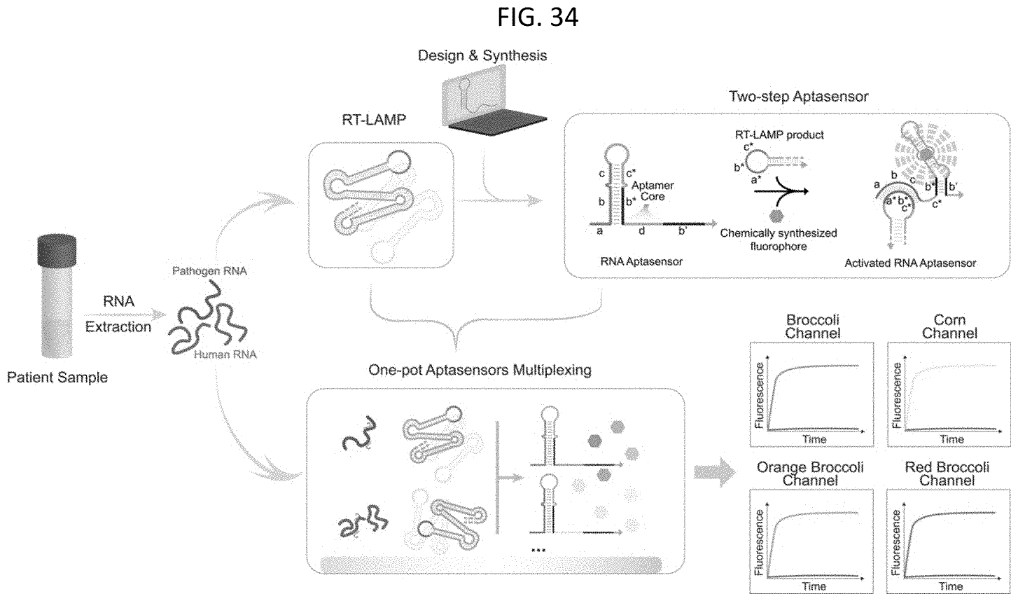

One-pot diagnostic assays where all reaction steps occur in the same reaction vessel and do not require the additional reagents to be added after the start of the reaction are highly desirable. Such assays reduce processing time, time to result, and the likelihood of cross-contamination. Accordingly, we have developed one-pot RT-LAMP/aptasensor assays for detection of SARS-CoV-2 RNA. The one-pot assay process is schematically illustrated in FIG. 34. RNA from a patient sample is added to a reaction combining RT-LAMP components and the aptasensors and fluorogens responsible for target detection. The reaction vessel is first heated to between 60° C. and 65° C. for 10 to 60 minutes to amplify the genetic material from the pathogen and control nucleic acids expected in a human sample. Following amplification, the temperature of the system is reduced enabling binding of the aptasensors that generate a fluorescence signal indicating the presence of their respective target nucleic acids. Owing to the generalizability of the aptasensors, multiple aptamers can serve as outputs for the assay enabling multiplexed output with multiple fluorescence profiles (e.g. Broccoli, Corn, Red Broccoli, and Orange Broccoli as illustrated).

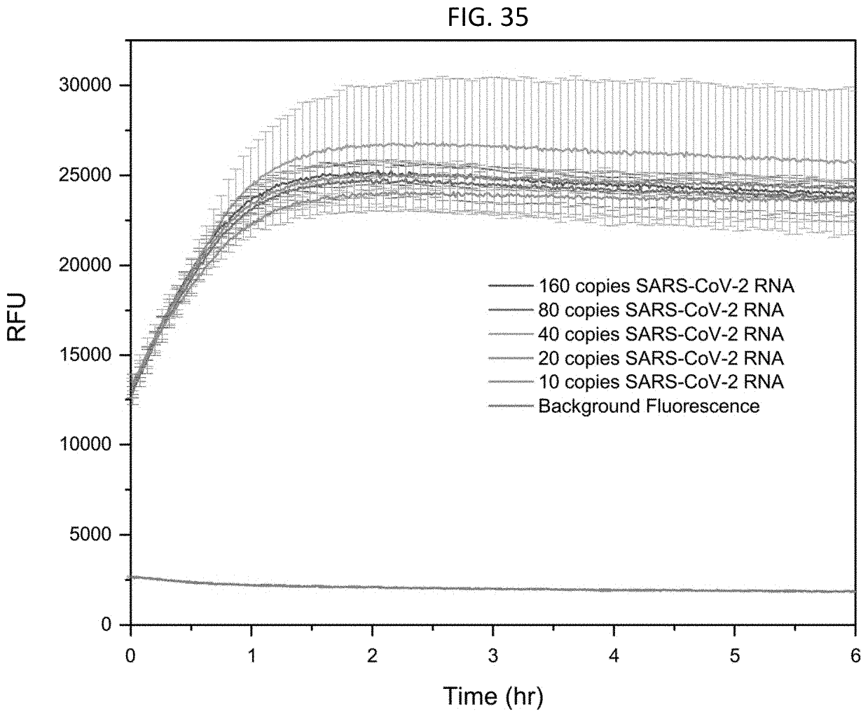

Results from a one-channel reaction are shown in FIG. 35 (SEQ ID NOs:121 and 143; see Table 26). This one-pot reaction combines all RT-LAMP components (enzymes, primers, and buffer) along with two Broccoli aptasensors (0.5 μM each) targeting two loops of the RT-LAMP amplicon of the SARS-CoV-2 N gene. Primers were added at 1.5× the standard concentrations for RT-LAMP (i.e., 2.4 μM for FIP and BIP, 0.3 μM for F3 and B3, and 0.6 μM for LF and LB primers). The reaction also includes the fluorogen BI at a concentration of 2 μM and KCl at 40 mM. Following addition of the SARS-CoV-2 sample, the reaction is incubated in a plate in a temperature-controlled plate reader that provides the Broccoli/BI fluorescence readout in real time or incubated in a thermal cycler or incubator. A 45-minute amplification step at 61° C. is used for the RT-LAMP reaction and followed by cooling to at 37° C. temperature. Over this second stage, the reduced temperature enables the aptasensors to bind to the RT-LAMP amplicons and the BI fluorophore. A strong and rapid increase in Broccoli fluorescence is observed in the reaction signaling the presence of the SARS-CoV-2 N gene. The assay enables detection down to 10 copies of SARS-CoV-2 RNA, corresponding to a concentration of 0.5 copies/μL or 1 aM.

In other cases, the one-pot assay is performed with lx concentrations of the RT-LAMP primers (i.e., 1.6 μM for FIP and BIP, 0.2 μM for F3 and B3, and 0.4 μM for LF and LB primers). In some cases, the one-pot assay is performed with no ions added with 2 μM of BI. In other cases, 2 μM of BI is used with 40 mM KI and 1 mM MgCl₂. In some cases, the assay is performed with only one aptasensor provided at a concentration of 0.5 μM.

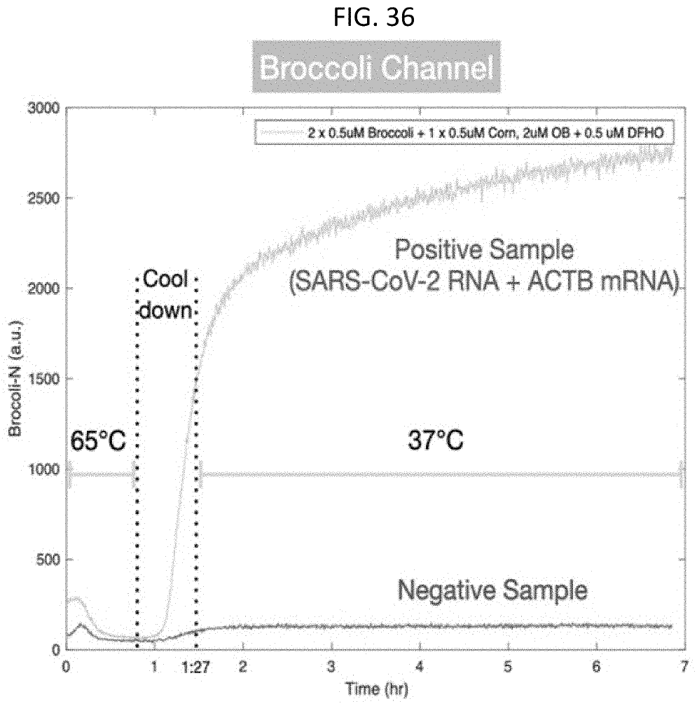

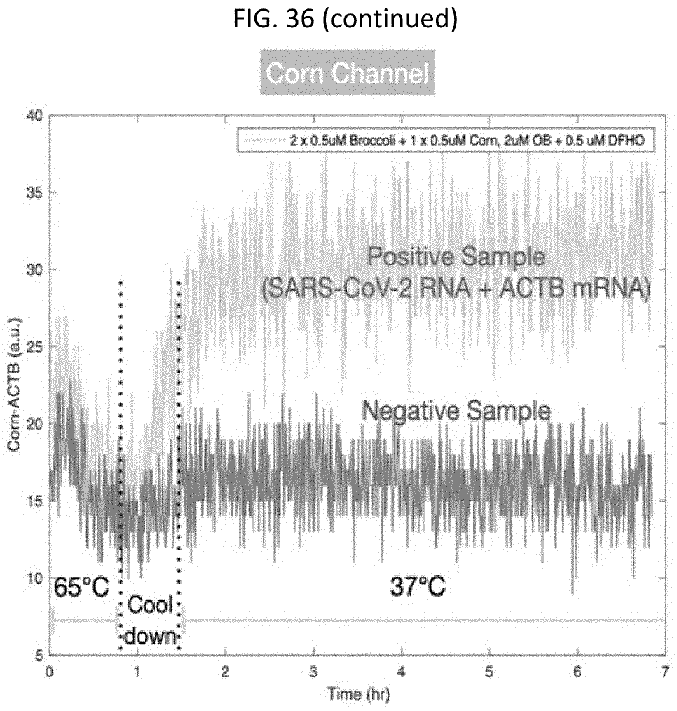

We next exploited the multiplexing capabilities of our aptasensor systems by implementing a two-channel, one-pot RT-LAMP/aptasensor assay. This assay again combines RT-LAMP components with aptasensor reagents. Primers for both the SARS-CoV-2 N gene and the ACTB mRNA control were provided at 1.5× the standard RT-LAMP concentration. The fluorogens BI and DFHO were present at 2 μM and 0.5 μM, respectively. No additional ions were added. The SARS-CoV-2 N gene was targeted with dual Broccoli aptasensors at 0.5 μM concentration, while a single Corn aptasensor at 0.5 μM was used for detection of ACTB mRNA. FIG. 36 shows the real-time changes in the fluorescence of the Broccoli and Corn aptasensors over the course of the one-pot reaction in a temperature-controlled plate reader (SEQ ID NOs:143 and 247-248; see Table 27). RT-LAMP is initially performed at 65° C. for 45 minutes, followed by a cooling period where the plate reader equilibrates to 37° C. Reduction of temperature enables binding of the aptasensors and is evidenced by a rapid increase in the fluorescence of both Broccoli and Corn, signifying the presence of both SARS-CoV-2 N gene and the ACTB mRNA. Negative samples lacking both targets displayed low fluorescence signals as expected.

### Validation of Assay Against Clinical Samples using the Two-Channel, Two-pot RT-LAMP/Aptasensor Assay

The general SARS-CoV-2 detection assay developed by our lab requires the following steps. A patient saliva sample is subjected to viral RNA extraction and amplified by reverse transcription loop-mediated isothermal amplification (RT-LAMP). The amplified product is then detected using aptamer-based sensors referred to as aptasensors, which are designed to detect SARS-CoV-2 genetic material and the human actin mRNA sequence. The latter transcript serves a sample control to ensure proper sample processing. The computer-designed aptasensors recognize specific target sequences and form the active structure of the output aptamer only after binding to the viral target RNA. Fluorescence from the aptasensors is generated when a non-fluorescent dye ligand interacts with the aptamer and generates a fluorescently active conformation once bound to the aptamer binding site. The material costs for the assay are approximately $4/test when detecting one SARS-CoV-2 target and the actin control mRNA.

Following FDA EUA requirements, we have validated the assay using a panel of 30 positive and 30 negative patient samples. These samples were obtained from the Biodesign Institute clinical testing laboratory and were provided by patients as saliva samples. RNA from the samples was first extracted using a PureLink RNA extraction kit and SARS-CoV-2 RNA concentrations quantified via RT-qPCR using the TaqMan 2019-nCoV Assay Kit. The extracted RNA was supplied to the RT-LAMP reactions and incubated at 61° C. for 45 minutes. During the amplification, SARS-CoV-2 RNA in the spike gene was amplified at the same time as the actin control mRNA. Following RT-LAMP, the DNA product was then added to a mixture containing a Broccoli aptasensor for the spike gene, a Corn aptasensor for actin mRNA, and the fluorogenic dyes DFHBI-1T for Broccoli and DFHO for Corn. This readout reaction was then measured in 384-well plates in a plate reader at 37° C. while monitoring the green and yellow fluorescence from the Broccoli and Corn aptasensors, respectively.

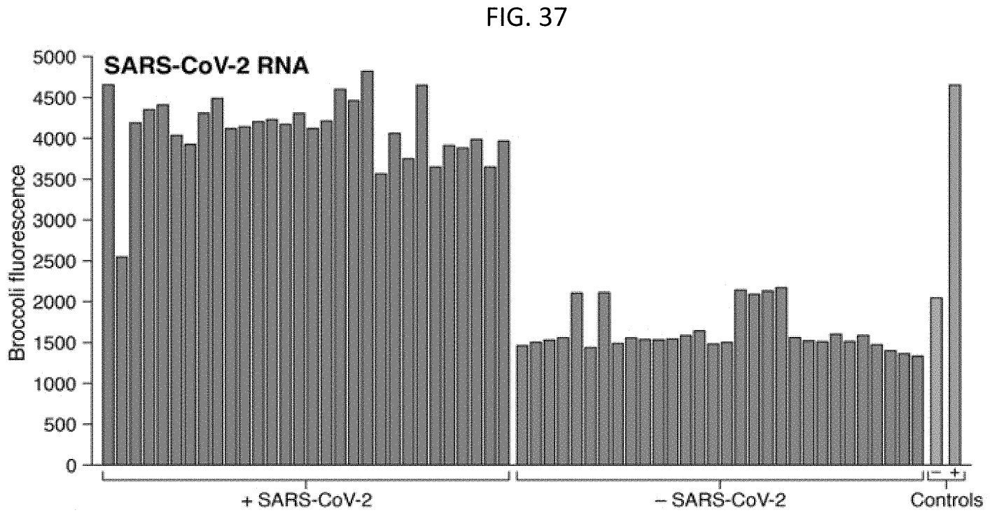

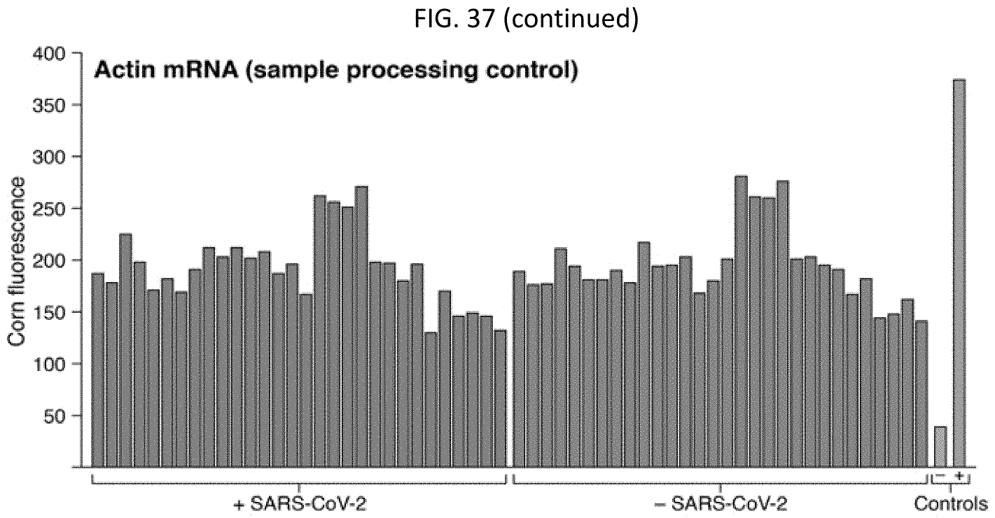

FIG. 37 shows the fluorescence of the Broccoli (SEQ ID NO:121; see Table 25) and Corn (SEQ ID NO:143; see Table 25) aptasensors after 15 minutes. In general, the SARS-CoV-2 positive samples display a much higher Broccoli fluorescence than the negative samples as expected, while the Corn fluorescence is similar across all the samples. Based on previous calibration experiments, we applied a Broccoli fluorescence threshold of 3000 for identifying positive samples after 15 minutes of incubation, along with a Corn fluorescence of 120 for properly processed samples. These criteria identified 29 out of 30 positive samples correctly, while 30 out of 30 negative samples were correctly determined to be free of SARS-CoV-2 RNA. Accordingly, the sensitivity of this assay is 96.67% and the specificity is 100%. The accuracy is 98.33%. The assay successfully detected SARS-CoV-2 from clinical samples down to concentrations as low as 0.31 copies/μL in the RT-LAMP reaction (6.2 copies/μL in the extracted sample).

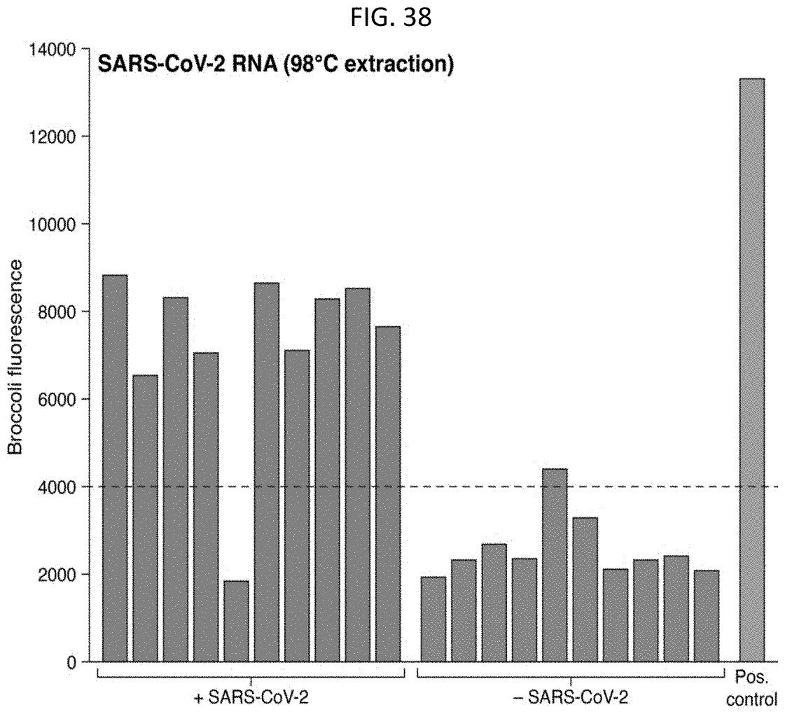

We investigated multiple fast extraction methods for isolating viral RNA from saliva samples, including protease K treatments, Triton X-100 processing, and use of ARCIS reagents. From these studies, we found that the simplest method, a brief 5-minute heating step at 98° C., provided the best combination of extraction speed and assay results. We then applied the 98° C. extraction method to a panel of 10 positive and 10 negative clinical saliva samples. Aliquots of 12 μL of the saliva sample were heated at 98° C. for 5 minutes and 1.5 μL of the resulting product was added to RT-LAMP reactions at a final volume of 30 μL. After incubation for 45 minutes at 61° C., the RT-LAMP product was added the aptasensor/dye solution for readout. FIG. 38 shows the Broccoli fluorescence from the heat-extracted saliva samples. Overall, the assay achieved 90% sensitivity, 90% specificity, and 90% accuracy. We expect that these metrics can be improved with further optimization of reaction conditions and extraction conditions.

To increase the throughput and ease of implementation of the tests, we have implemented a streamlined approach for 384-well assay processing. Since the assay employs two separate reaction steps, RT-LAMP and aptasensor readout, we developed master mix formulations for reactions that can be stably stored at −20° C. and rapidly added to 384-well plates at the time of use. For the RT-LAMP reactions, the master mix contains the RT-LAMP enzyme, buffer, and primers. It can be stored for multiple weeks in the freezer and remain active. Moreover, the mix can be provided pre-aliquoted into the wells of a 384-well plate and stored at −20° C. Upon thawing, the reactions can be started in the plate immediately after addition of the RNA sample to each well. For the aptasensor readout reactions, master mixes containing the aptasensor RNAs, buffer, DFHBI-1T, and DFHO also remained stable under -20° C. storage and could be aliquoted into a 384-well plate prior to measurement. In addition to the master mix formulations, we also optimized the assay by reducing the RT-LAMP step from 45 minutes to 30 minutes without affecting assay sensitivity.

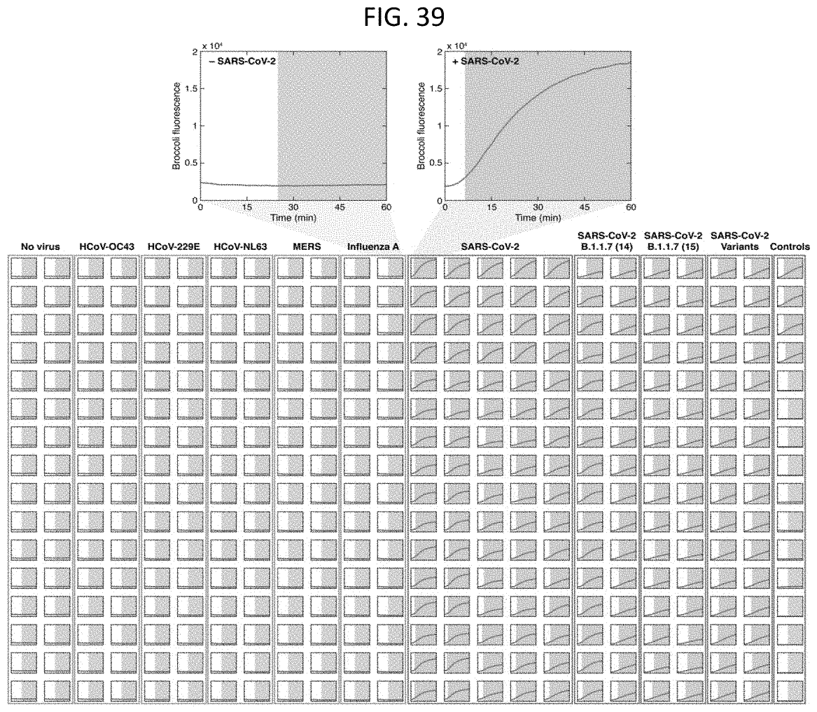

Results from the parallelized 384-well assay are shown in FIG. 39. Contrived samples for the high-throughput testing were prepared from extracted RNA preparations using concentrations typical of clinical saliva samples. The upper part of the figure shows the time course measurements of Broccoli fluorescence for a representative pair of positive and negative SARS-CoV-2 wells. The green shaded region indicates the time point at which the Broccoli fluorescence passed the fluorescence threshold for samples identified as positive. In contrast, the gray region indicates the time at which a sample is identified as negative. More specifically, samples were deemed negative for SARS-CoV-2 if they did not cross the Broccoli fluorescence threshold within 25 minutes of incubation. The lower part of FIG. 39 shows the Broccoli fluorescence readouts from all 384 wells on the plate. Control samples occupy the far-right column, while negative samples occupy the left half of the plate. The remaining wells, occupying most of the right side of the plate, contain RNA from different SARS-CoV-2 strains.

Analysis of the Broccoli fluorescence curves and Corn fluorescence from the plate revealed that the high-throughput assay was highly effective at identifying SARS-CoV-2. Of the positive samples, 176 out of 176 were correctly identified. Similarly, 192 out of 192 negative samples were correctly assigned. In the same plate, we also tested the specificity of the assay against potential confounding viruses, in particular multiple other human coronaviruses and influenza. These confounding samples showed no activation of the Broccoli aptasensor, further demonstrating the excellent specificity of the SARS-CoV-2 test.

### TABLES

TABLE 1
Sequences of the Broccoli RNA aptasensors tested in FIG. 3

| Name | Sequence |
|---|---|
| broc_rot_arb_ | GGGUUAACAUAUAGUGAACCGCCACACAUGACCAUUUCUGUAUCUUGA |
| b07_covid19_ | AUUGGACAUGUGUGGCGGUCGAGUAGAGUGUGGGCUCAGAUUCGUCUG |
| RdRP_A | AGACGGUCGGGUCCCGCCAC (SEQ ID NO: 1) |
| broc_rot_arb_ | GGGUAACAUAUAGUGAACCGCCACACAUGACCAUUUCAACCUAAUCUG |
| b07_covid19_ | AUAUGAUCAUGUGUGGCGUCGAGUAGAGUGUGGGCUCAGAUUCGUCUG |
| RdRP_B | AGACGGUCGGGUCCGCCACA (SEQ ID NO: 2) |
| broc_rot_arb_ | GGGUCUCCUGAUGAGGUUCCACCUGGUUUAACAUAUAGUCAAAUCCCU |
| b08_covid19_ | AAAUGAUAAACCAGGUGGUCGAGUAGAGUGUGGGCUCAGAUUCGUCUG |
| RdRP_A | AGACGGUCGGGUCCCACCUGG (SEQ ID NO: 3) |
| broc_rot_arb_ | GGGAGAUAAAAGUGCAUUAACAUUGGCCGUGACAGCUUAAUACCCGAA |
| b08_covid19_ | GAUGUGACGGCCAAUGUUUCGAGUAGAGUGUGGGCUCAGAUUCGUCUG |
| RdRP_B | AGACGGUCGGGUCAACAUUGG (SEQ ID NO: 4) |
| broc_rot_arb_ | GGGUUAACAUAUAGUGAACCGCCACACAUGACCAUUUCUACGCACUGA |
| b09_covid19_ | AUUGGCCAUGUGUGGCGGUCGAGUAGAGUGUGGGCUCAGAUUCGUCUG |
| RdRP_A | AGACGGUCGGGUCCCGCCACAC (SEQ ID NO: 5) |
| broc_rot_arb_ | GGGAGAUAAAAGUGCAUUAACAUUGGCCGUGACAGCUUAAUACUCCAA |
| b09_covid19_ | GGUGUAACGGCCAAUGUUUCGAGUAGAGUGUGGGCUCAGAUUCGUCUG |
| RdRP_B | AGACGGUCGGGUCAACAUUGGC (SEQ ID NO: 6) |
| broc_rot_arb_ | GGGUUUAACAUAUAGUGAACCGCCACACAUGACCAUUUACAACAUCAA |
| b10_covid19_ | ACGGUGAUGUGUGGCGGUUCGAGUAGAGUGUGGGCUCAGAUUCGUCUG |
| RdRP_A | AGACGGUCGGGUCACCGCCACAC (SEQ ID NO: 7) |
| broc_rot_arb_ | GGGAUAAAAGUGCAUUAACAUUGGCCGUGACAGCUUGAAAUGUAGCAA |
| b10_covid19_ | ACUGGCACGGCCAAUGUUCGAGUAGAGUGUGGGCUCAGAUUCGUCUGA |
| RdRP_B | GACGGUCGGGUCACAUUGGCCG (SEQ ID NO: 8) |

| Name | Sequence |
|---|---|
| broc_rot_arb_ | GGGAAUAGUUAAUAGCGUACUUCUUUUUCUUGCUUUCGACUCUUCACG |
| b07_covid19rev_ | ACAGCUAGAAAAAGAAGUUCGAGUAGAGUGUGGGCUCAGAUUCGUCUG |
| E_A | AGACGGUCGGGUCACUUCUU (SEQ ID NO: 9) |
| broc_rot_arb_ | GGGCGCUUCGAUUGUGUGCGUACUGCUGCAAUAUUGUUAUCAUCCAAA |
| b07_covid19rev_ | CUAUAAUGCAGCAGUACGUCGAGUAGAGUGUGGGCUCAGAUUCGUCUG |
| E_B | AGACGGUCGGGUCCGUACUG (SEQ ID NO: 10) |
| broc_rot_arb_ | GGGCGCUUCGAUUGUGUGCGUACUGCUGCAAUAUUGUUAGAUUAAGAA |
| b08_covid19rev_ | CCAUAAUGCAGCAGUACGUCGAGUAGAGUGUGGGCUCAGAUUCGUCUG |
| E_A | AGACGGUCGGGUCCGUACUGC (SEQ ID NO: 11) |
| broc_rot_arb_ | GGGAAUAGUUAAUAGCGUACUUCUUUUUCUUGCUUUCGUUAGAUCUCG |
| b08_covid19rev_ | AUAGCCAGAAAAAGAAGUUCGAGUAGAGUGUGGGCUCAGAUUCGUCUG |
| E_B | AGACGGUCGGGUCACUUCUUU (SEQ ID NO: 12) |
| broc_rot_arb_ | GGGCGUUAAUAGUUAAUAGCGUACUUCUUUUUCUUGCUGCCCGUUAAG |
| b09_covid19rev_ | CCAGAUAAAGAAGUACGCUCGAGUAGAGUGUGGGCUCAGAUUCGUCUG |
| E_A | AGACGGUCGGGUCGCGUACUUC (SEQ ID NO: 13) |
| broc_rot_arb_ | GGGUUACACUAGCCAUCCUUACUGCGCUUCGAUUGUGUCCACCCUCACU |
| b09_covid19rev_ | AUCCAAGCGCAGUAAGUCGAGUAGAGUGUGGGCUCAGAUUCGUCUGAG |
| E_B | ACGGUCGGGUCCUUACUGCG (SEQ ID NO: 14) |
| broc_rot_arb_ | GGGAUAGUUAAUAGCGUACUUCUUUUUCUUGCUUUCGUUCGACCAUAC |
| b10_covid19rev_ | GUAAGCAAGAAAAAGAAGUCGAGUAGAGUGUGGGCUCAGAUUCGUCUG |
| E_A | AGACGGUCGGGUCCUUCUUUUUC (SEQ ID NO: 15) |
| broc_rot_arb_ | GGGACACUAGCCAUCCUUACUGCGCUUCGAUUGUGUGCAAACUAAGGC |
| b10_covid19rev_ | AAACACUCGAAGCGCAGUUCGAGUAGAGUGUGGGCUCAGAUUCGUCUG |
| E_B | AGACGGUCGGGUCACUGCGCUUC (SEQ ID NO: 16) |

TABLE 3
Sequences of the Broccoli RNA aptasensors tested in FIG. 5

| Name | Sequence |
|---|---|
| 3.1 FIG. 5A | |
| broc_rot_arb_b07_ | GGGAUGUGCAUUACCAUGGACUUGACAAUACAGAUCAUUGAUAGGU |
| covid19_ORF16_ | AUGCUCUGUAUUGUCAAGUCUCGAGUAGAGUGUGGGCUCAGAUUCG |
| A | UCUGAGACGGUCGGGUCGACUUGA (SEQ ID NO: 17) |
| broc_rot_arb_b07_ | GGGUAUUCAAUAGUCCAGUCAACACGCUUAACAAAGCAUACCGUCUU |
| covid19_ORF16_ | GCCUUGCUAAGCGUGUUGAUCGAGUAGAGUGUGGGCUCAGAUUCGUC |
| B | UGAGACGGUCGGGUCUCAACAC (SEQ ID NO: 18) |
| broc_rot_arb_b08_ | GGGUAUUCAAUAGUCCAGUCAACACGCUUAACAAAGCAGAACCUAAU |
| covid19_ORF1b_ | GCCUUGCUAAGCGUGUUGAUCGAGUAGAGUGUGGGCUCAGAUUCGUC |
| A | UGAGACGGUCGGGUCUCAACACG (SEQ ID NO: 19) |
| broc_rot_arb_b08_ | GGGAUGUGCAUUACCAUGGACUUGACAAUACAGAUCAUUCAGUUCUA |
| covid19_ORF1b_ | UGUUCUGUAUUGUCAAGUCUCGAGUAGAGUGUGGGCUCAGAUUCGU |
| B | CUGAGACGGUCGGGUCGACUUGAC (SEQ ID NO: 20) |
| broc_rot_arb_b09_ | GGGAGGAUAUUCAAUAGUCCAGUCAACACGCUUAACAAUCACUUCCU |
| covid19_ORF1b_ | UGCUAACCGUGUUGACUGGUCGAGUAGAGUGUGGGCUCAGAUUCGUC |
| A | UGAGACGGUCGGGUCCCAGUCAAC (SEQ ID NO: 21) |
| broc_rot_arb_b09_ | GGGUGGACAGCUAGACACCUAGUCAUGAUUGCAUCACUUGUCUUUGU |
| covid19_ORF1b_ | GCUGCCAUCAUGACUAGGUCGAGUAGAGUGUGGGCUCAGAUUCGUCU |
| B | GAGACGGUCGGGUCCCUAGUCAU (SEQ ID NO: 22) |
| broc_rot_arb_b10_ | GGGAGGAUAUUCAAUAGUCCAGUCAACACGCUUAACAAGAAUCAAAU |
| covid19_ORF1b_ | UGCUAAUCGUGUUGACUGGUCGAGUAGAGUGUGGGCUCAGAUUCGU |
| A | CUGAGACGGUCGGGUCCCAGUCAACA (SEQ ID NO: 23) |
| broc_rot_arb_b10_ | GGGUGGACAGCUAGACACCUAGUCAUGAUUGCAUCACUUACACUUGU |
| covid19_ORF1b_ | GCUGCUAUCAUGACUAGGUCGAGUAGAGUGUGGGCUCAGAUUCGUCU |
| B | GAGACGGUCGGGUCCCUAGUCAUG (SEQ ID NO: 24) |
| 3.2 FIG. 5B | |
| broc_rot_arb_b07_ | GGGUGUUUGUAAUCAGUUCCUUGUCUGAUUAGUUCCUGGACCAGAAC |
| covid19_N_ | AGUAACCAAUCAGACAAGGUCGAGUAGAGUGUGGGCUCAGAUUCGUC |
| A | UGAGACGGUCGGGUCCCUUGUC (SEQ ID NO: 25) |
| broc_rot_arb_b07_ | GGGUUGUCUGAUUAGUUCCUGGUCCCCAAAAUUUCCUUGAUCAUGAA |
| covid19_N_ | AGCAAACUUUGGGGACCAGUCGAGUAGAGUGUGGGCUCAGAUUCGUC |
| B | UGAGACGGUCGGGUCCUGGUCC (SEQ ID NO: 26) |
| broc_rot_arb_b08_ | GGGUGUUUGUAAUCAGUUCCUUGUCUGAUUAGUUCCUGGCCUAUUAC |
| covid19_N_ | AGCAACCAAUCAGACAAGGUCGAGUAGAGUGUGGGCUCAGAUUCGUC |
| A | UGAGACGGUCGGGUCCCUUGUCU (SEQ ID NO: 27) |
| broc_rot_arb_b08_ | GGGUAGGUCAACCACGUUCCCGAAGGUGUGACUUCCAUGGUCCUGAA |
| covid19_N_ | UGCAAGACACACCUUCGGGUCGAGUAGAGUGUGGGCUCAGAUUCGUC |
| B | UGAGACGGUCGGGUCCCCGAAGG (SEQ ID NO: 28) |
| broc_rot_arb_b09_ | GGGUGUUUGUAAUCAGUUCCUUGUCUGAUUAGUUCCUGAACGUAACC |
| covid19_N_ | AGUAACAAAUCAGACAAGGUCGAGUAGAGUGUGGGCUCAGAUUCGU |
| A | CUGAGACGGUCGGGUCCCUUGUCUG (SEQ ID NO: 29) |
| broc_rot_arb_b09_ | GGGUAGGUCAACCACGUUCCCGAAGGUGUGACUUCCAUUCUGCCCUA |
| covid19_N_ | UGUAAGCCACACCUUCGGGUCGAGUAGAGUGUGGGCUCAGAUUCGUC |
| B | UGAGACGGUCGGGUCCCCGAAGGU (SEQ ID NO: 30) |
| broc_rot_arb_b10_ | GGGUGUUUGUAAUCAGUUCCUUGUCUGAUUAGUUCCUGCCACUCCAC |
| covid19_N_ | AGUAACCAAUCAGACAAGGUCGAGUAGAGUGUGGGCUCAGAUUCGUC |
| A | UGAGACGGUCGGGUCCCUUGUCUGA (SEQ ID NO: 31) |
| broc_rot_arb_b10_ | GGGUUGUCUGAUUAGUUCCUGGUCCCCAAAAUUUCCUUGAACAUAAA |
| covid19_N_ | AGCAAACUUUGGGGACCAGUCGAGUAGAGUGUGGGCUCAGAUUCGUC |
| B | UGAGACGGUCGGGUCCUGGUCCCCA (SEQ ID NO: 32) |
| 3.3 FIG. 5C | |
| broc_rot_arb_b07_ | GGGUAACUAUUAACGUACCUGUCUCUUCCGAAACGAAUAGACCGGAA |
| covid19_E_ | UUCGUUCCGGAAGAGACAGUCGAGUAGAGUGUGGGCUCAGAUUCGUC |
| A | UGAGACGGUCGGGUCCUGUCUC (SEQ ID NO: 33) |
| broc_rot_arb_b07_ | GGGUAACUAGCAAGAAUACCACGAAAGCAAGAAAAAGUCUAGUAUC |
| covid19_E_ | UUAUUCAUGCUUUCGUGGUUCGAGUAGAGUGUGGGCUCAGAUUCGU |
| B | CUGAGACGGUCGGGUCACCACGA (SEQ ID NO: 34) |
| broc_rot_arb_b08_ | GGGUUAACUAUUAACGUACCUGUCUCUUCCGAAACGAAGCCGCAAAU |
| covid19_E_ | UCAUUUCGGAAGAGACAGGUCGAGUAGAGUGUGGGCUCAGAUUCGU |
| A | CUGAGACGGUCGGGUCCCUGUCUC (SEQ ID NO: 35) |
| broc_rot_arb_b08_ | GGGUAACUAGCAAGAAUACCACGAAAGCAAGAAAAAGUACCUAAUCU |
| covid19_E_ | UAUUCAUGCUUUCGUGGUUCGAGUAGAGUGUGGGCUCAGAUUCGUC |
| B | UGAGACGGUCGGGUCACCACGAA (SEQ ID NO: 36) |
| broc_rot_arb_b09_ | GGGCGAAAGCAAGAAAAAGAAGUACGCUAUUAACUAUUGGAGUAGA |
| covid19_E_ | AAUCGUUCAUAGCGUACUUCUCGAGUAGAGUGUGGGCUCAGAUUCGU |
| A | CUGAGACGGUCGGGUCGAAGUACGC (SEQ ID NO: 37) |
| broc_rot_arb_b09_ | GGGUAACUAGCAAGAAUACCACGAAAGCAAGAAAAAGAUUCCAACUU |
| covid19_E_ | CUAUUUCUUGCUUUCGUGGUCGAGUAGAGUGUGGGCUCAGAUUCGUC |
| B | UGAGACGGUCGGGUCCCACGAAAG (SEQ ID NO: 38) |
| broc_rot_arb_b10_ | GGGAUUGCAGCAGUACGCACACAAUCGAAGCGCAGUAAGAACAGAAU |
| covid19_E_ | UAGUGCACUUCGAUUGUGUUCGAGUAGAGUGUGGGCUCAGAUUCGU |
| A | CUGAGACGGUCGGGUCACACAAUCGA (SEQ ID NO: 39) |
| broc_rot_arb_b10_ | GGGUAACUAUUAACGUACCUGUCUCUUCCGAAACGAAUGGAGUGGAA |
| covid19_E_ | UUAGUUACGGAAGAGACAGUCGAGUAGAGUGUGGGCUCAGAUUCGU |
| B | CUGAGACGGUCGGGUCCUGUCUCUUC (SEQ ID NO: 40) |
| 3.4 FIG. 5D | |
| broc_rot_arb_b07_ | GGGUUGUUUUGAUCGCGCCCCACUGCGUUCUCCAUUCACUAAACCGA |
| covid19_cdc_ | ACGGAAAACGCAGUGGGGUCGAGUAGAGUGUGGGCUCAGAUUCGUC |
| n1_A | UGAGACGGUCGGGUCCCCCACU (SEQ ID NO: 41) |
| broc_rot_arb_b07_ | GGGUGGGGUCCAUUAUCAGACAUUUUAGUUUGUUCGUUUCACAAAU |
| covid19_cdc_ | AACUAACCAACUAAAAUGUCUCGAGUAGAGUGUGGGCUCAGAUUCGU |
| n1_B | CUGAGACGGUCGGGUCGACAUUU (SEQ ID NO: 42) |
| broc_rot_arb_b08_ | GGGUUGUUUUGAUCGCGCCCCACUGCGUUCUCCAUUCUGCAUCCAAA |
| covid19_cdc_ | GAUUGGCGAACGCAGUGGGUCGAGUAGAGUGUGGGCUCAGAUUCGU |
| n1_A | CUGAGACGGUCGGGUCCCCACUGC (SEQ ID NO: 43) |
| broc_rot_arb_b08_ | GGGACUGCGUUCUCCAUUCUGGUUACUGCCAGUUGAAUUUGAGGCUA |
| covid19_cdc_ | UUGAACCGGCAGUAACCAGUCGAGUAGAGUGUGGGCUCAGAUUCGUC |
| n1_B | UGAGACGGUCGGGUCCUGGUUAC (SEQ ID NO: 44) |
| broc_rot_arb_b09_ | GGGUUGUUUUGAUCGCGCCCCACUGCGUUCUCCAUUCUUUGAAGUUA |
| covid19_cdc_ | GAUUGGUGAACGCAGUGGGUCGAGUAGAGUGUGGGCUCAGAUUCGU |
| n1_A | CUGAGACGGUCGGGUCCCCACUGCG (SEQ ID NO: 45) |
| broc_rot_arb_b09_ | GGGUAAACCUUGGGGCCGACGUUGUUUUGAUCGCGGACCAAAACGCU |
| covid19_cdc_ | AUCUAAACAACGUCGGUCGAGUAGAGUGUGGGCUCAGAUUCGUCUGA |
| n1_B | GACGGUCGGGUCCCGACGUUG (SEQ ID NO: 46) |
| broc_rot_arb_b10_ | GGGUUGUUUUGAUCGCGCCCCACUGCGUUCUCCAUUCUCCAGUACAA |
| covid19_cdc_ | GAUUGGCGAACGCAGUGGGUCGAGUAGAGUGUGGGCUCAGAUUCGU |
| n1_A | CUGAGACGGUCGGGUCCCCACUGCGU (SEQ ID NO: 47) |
| broc_rot_arb_b10_ | GGGACUGCGUUCUCCAUUCUGGUUACUGCCAGUUGAAUGAGCAAGGA |
| covid19_cdc_ | UUGAACGGGCAGUAACCAGUCGAGUAGAGUGUGGGCUCAGAUUCGUC |
| n1_B | UGAGACGGUCGGGUCCUGGUUACUG (SEQ ID NO: 48) |

TABLE 4
Sequences of the Broccoli RNA aptasensors tested in FIG. 6

| Name | Sequence |
|---|---|
| 4.1 FIG. 6A | |
| broc_rot_arb_b07_ | GGGAGGAAGUUGUAGCACGAUUGCAGCAUUGUUAGCAGACGCCGAU |
| covid19_cdc_ | GCCAACUAUGCUGCAAUCGUCGAGUAGAGUGUGGGCUCAGAUUCGU |
| n3_A | CUGAGACGGUCGGGUCCGAUUGC (SEQ ID NO: 49) |
| broc_rot_arb_b07_ | GGGCGUAGAAGCCUUUUGGCAAUGUUGUUCCUUGAGGUCCAUCAUC |
| covid19_cdc_ | CUGAAGUAACAACAUUGCCUCGAGUAGAGUGUGGGCUCAGAUUCGU |
| n3_B | CUGAGACGGUCGGGUCGGCAAUG (SEQ ID NO: 50) |
| broc_rot_arb_b08_ | GGGAGGAAGUUGUAGCACGAUUGCAGCAUUGUUAGCAGUUGCCAUU |
| covid19_cdc_ | CUGGUAAGAAUGCUGCAAUCUCGAGUAGAGUGUGGGCUCAGAUUCG |
| n3_A | UCUGAGACGGUCGGGUCGAUUGCAG (SEQ ID NO: 51) |
| broc_rot_arb_b08_ | GGGUUUUGGCAAUGUUGUUCCUUGAGGAAGUUGUAGCAUUAGCACU |
| covid19_cdc_ | UGCAACAACUUCCUCAAGGAUCGAGUAGAGUGUGGGCUCAGAUUCG |
| n3_B | UCUGAGACGGUCGGGUCUCCUUGAG (SEQ ID NO: 52) |
| broc_rot_arb_bo9_ | GGGAUCUUUUGGUGUAUUCAAGGCUCCCUCAGUUGCAGGGGUCGAU |
| covid19_cdc_ | GCUACUUAGGGAGCCUUGAUCGAGUAGAGUGUGGGCUCAGAUUCGU |
| n3_A | CUGAGACGGUCGGGUCUCAAGGCUC (SEQ ID NO: 53) |
| broc_rot_arb_b09_ | GGGUUGAGGAAGUUGUAGCACGAUUGCAGCAUUGUUAGUCCAUCUU |
| covid19_cdc_ | CUACCAAGGCUGCAAUCGUGUCGAGUAGAGUGUGGGCUCAGAUUCG |
| n3_B | UCUGAGACGGUCGGGUCCACGAUUGC (SEQ ID NO: 54) |
| broc_rot_arb_b10_ | GGGAUUGUUAGCAGGAUUGCGGGUGCCAAUGUGAUCUUAUUCGGCA |
| covid19_cdc_ | AAGCUCAGAUUGGCACCCGCUCGAGUAGAGUGUGGGCUCAGAUUCG |
| n3_A | UCUGAGACGGUCGGGUCGCGGGUGCCA (SEQ ID NO: 55) |
| broc_rot_arb_b10_ | GGGAGGAAGUUGUAGCACGAUUGCAGCAUUGUUAGCAGCCUAACCU |
| covid19_cdc_ | CUGGUAAGAAUGCUGCAAUCUCGAGUAGAGUGUGGGCUCAGAUUCG |
| n3_B | UCUGAGACGGUCGGGUCGAUUGCAGCA (SEQ ID NO: 56) |
| 4.2 FIG. 6B | |
| broc_rot_arb_b07_ | GGGAAUCCGUUUAUGAUUGAUGUUCAACAAUGGGGUUUAACAUACC |
| covid19_rev_ | AAAGCCCGUUGUUGAACAUCUCGAGUAGAGUGUGGGCUCAGAUUCG |
| ORF1b_A | UCUGAGACGGUCGGGUCGAUGUUC (SEQ ID NO: 57) |
| broc_rot_arb_b07_ | GGGUUGUUAAGCGUGUUGACUGGACUAUUGAAUAUCCUGUUAGUUA |
| covid19rev_ | AGGCUAUACAAUAGUCCAGUUCGAGUAGAGUGUGGGCUCAGAUUCG |
| ORF1b_B | UCUGAGACGGUCGGGUCACUGGAC (SEQ ID NO: 58) |
| broc_rot_arb_b08_ | GGGUUUGUUAAGCGUGUUGACUGGACUAUUGAAUAUCCACAGGCAA |
| covid19rev_ | GGACAUUGAAUAGUCCAGUCUCGAGUAGAGUGUGGGCUCAGAUUCG |
| ORF1b_A | UCUGAGACGGUCGGGUCGACUGGAC (SEQ ID NO: 59) |
| broc_rot_arb_b08_ | GGGUUUUACAGGUAACCUACAAAGCAACCAUGAUCUGUAUGGUAAG |
| covid19rev_ | AACAUAGUUGCUUUGUAGUCGAGUAGAGUGUGGGCUCAGAUUCGUC |
| ORF1b_B | UGAGACGGUCGGGUCCUACAAAG (SEQ ID NO: 60) |
| broc_rot_arb_b09_ | GGGAAUCCGUUUAUGAUUGAUGUUCAACAAUGGGGUUUCACUAUAC |
| covid19rev_ | AAAGCCCGUUGUUGAACAUCUCGAGUAGAGUGUGGGCUCAGAUUCG |
| ORF1b_A | UCUGAGACGGUCGGGUCGAUGUUCAA (SEQ ID NO: 61) |
| broc_rot_arb_b09_ | GGGUUGUUAAGCGUGUUGACUGGACUAUUGAAUAUCCUAACGAACC |
| covid19rev_ | AGGCUAUACAAUAGUCCAGUUCGAGUAGAGUGUGGGCUCAGAUUCG |
| ORF1b_B | UCUGAGACGGUCGGGUCACUGGACUA (SEQ ID NO: 62) |
| broc_rot_arb_b10_ | GGGUUUGUUAAGCGUGUUGACUGGACUAUUGAAUAUCCGUGCAUUA |
| covid19rev_ | GGACAUUGAAUAGUCCAGUCUCGAGUAGAGUGUGGGCUCAGAUUCG |
| ORF1b_A | UCUGAGACGGUCGGGUCGACUGGACUA (SEQ ID NO: 63) |
| broc_rot_arb_b10_ | GGGAAUCCGUUUAUGAUUGAUGUUCAACAAUGGGGUUUACAUGCAC |
| covid19rev_ | AAAGCCCGUUGUUGAACAUCUCGAGUAGAGUGUGGGCUCAGAUUCG |
| ORF1b_B | UCUGAGACGGUCGGGUCGAUGUUCAAC (SEQ ID NO: 64) |
| 4.3 FIG. 6C | |
| broc_rot_arb_b07_ | GGGAACUGAUUACAAACAUUGGCCGCAAAUUGCACAUGGUAGAUUG |
| covid19rev_ | UACAAAUUGCGGCCAAUGUCGAGUAGAGUGUGGGCUCAGAUUCGUC |
| N_A | UGAGACGGUCGGGUCCAUUGGC (SEQ ID NO: 65) |
| broc_rot_arb_b07_ | GGGAACUAAUCAGACAAGGAACUGAUUACAAACAUUGGAAAUGGCA |
| covid19rev_ | CCACUGUCUGUAAUCAGUUCUCGAGUAGAGUGUGGGCUCAGAUUCG |
| N_B | UCUGAGACGGUCGGGUCGAACUGA (SEQ ID NO: 66) |
| broc_rot_arb_b08_ | GGGAGGAACUGAUUACAAACAUUGGCCGCAAAUUGCACGGUGCGAA |
| covid19rev_ | GUGGAAUGUGCGGCCAAUGUUCGAGUAGAGUGUGGGCUCAGAUUCG |
| N_A | UCUGAGACGGUCGGGUCACAUUGGC (SEQ ID NO: 67) |
| broc_rot_arb_b08_ | GGGAGGAAAUUUUGGGGACCAGGAACUAAUCAGACAAGAAUCAACG |
| covid19rev_ | CUUAUCUUAUUAGUUCCUGGUCGAGUAGAGUGUGGGCUCAGAUUCG |
| N_B | UCUGAGACGGUCGGGUCCCAGGAAC (SEQ ID NO: 68 |
| broc_rot_arb_b09_ | GGGACAAGGAACUGAUUACAAACAUUGGCCGCAAAUUGAAAGUGGA |
| covid19rev_ | AUAUGCUGCCAAUGUUUGUUCGAGUAGAGUGUGGGCUCAGAUUCGU |
| N_A | CUGAGACGGUCGGGUCACAAACAUU (SEQ ID NO: 69) |
| broc_rot_arb_b09_ | GGGAAAUUUUGGGGACCAGGAACUAAUCAGACAAGGUCCUGCUUCC |
| covid19rev_ | UAGUCGGAUUAGUUCCUGUCGAGUAGAGUGUGGGCUCAGAUUCGUC |
| N_B | UGAGACGGUCGGGUCCAGGAACUA (SEQ ID NO: 70) |
| broc_rot_arb_b10_ | GGGUGGUCCAGAACAAACCCAAGGAAAUUUUGGGGACCGUGCAGGA |
| covid19rev_ | GGUGCCCUAAAUUUCCUUGGUCGAGUAGAGUGUGGGCUCAGAUUCG |
| N_A | UCUGAGACGGUCGGGUCCCAAGGAAAU (SEQ ID NO: 71) |
| broc_rot_arb_b10_ | GGGAAUUUUGGGGACCAGGAACUAAUCAGACAAGGAACUACAAAAC |
| covid19rev_ | GUUGCUUAUCUGAUUAGUUCUCGAGUAGAGUGUGGGCUCAGAUUCG |
| N_B | UCUGAGACGGUCGGGUCGAACUAAUCA (SEQ ID NO: 72) |
| 4.4 FIG. 6D | |
| broc_rot_arb_b07_ | GGGAUGUUAAACCAGGUGGAACCUCAUCAGGAGAUGCCAACGAUCA |
| covid19rev_ | GGCUUCUGCUGAUGAGGUUCUCGAGUAGAGUGUGGGCUCAGAUUCG |
| RdRP_A | UCUGAGACGGUCGGGUCGAACCUC (SEQ ID NO: 73) |
| broc_rot_arb_b07_ | GGGAUGGUCAUGUGUGGCGGUUCACUAUAUGUUAAACCUUUGUCCU |
| covid19rev_ | GGUCUAAGAUAUAGUGAACCUCGAGUAGAGUGUGGGCUCAGAUUCG |
| RdRP_B | UCUGAGACGGUCGGGUCGGUUCAC (SEQ ID NO: 74) |
| broc_rot_arb_b08_ | GGGAAAUGGUCAUGUGUGGCGGUUCACUAUAUGUUAAACUCAUCCU |
| covid19rev_ | UAUCAUUUAGUGAACCGCCUCGAGUAGAGUGUGGGCUCAGAUUCGU |
| RdRP_A | CUGAGACGGUCGGGUCGGCGGUUC (SEQ ID NO: 75) |
| broc_rot_arb_b08_ | GGGUCACUAUAUGUUAAACCAGGUGGAACCUCAUCAGGUCCGGAUU |
| covid19rev_ | CCUAAUGCGGUUCCACCUGGUCGAGUAGAGUGUGGGCUCAGAUUCG |
| RdRP_B | UCUGAGACGGUCGGGUCCCAGGUGG (SEQ ID NO: 76) |
| broc_rot_arb_b09_ | GGGUAUAGAUUAGCUAAUGAGUGUGCUCAAGUAUUGAGGACAUUGA |
| covid19rev_ | CUCUAUAGUUGAGCACACUCUCGAGUAGAGUGUGGGCUCAGAUUCG |
| RdRP_A | UCUGAGACGGUCGGGUCGAGUGUGCU (SEQ ID NO: 77) |
| broc_rot_arb_b09_ | GGGAAAUGGUCAUGUGUGGCGGUUCACUAUAUGUUAACUCAAUUCU |
| covid19rev_ | UACCAUUUAGUGAACCGCCUCGAGUAGAGUGUGGGCUCAGAUUCGU |
| RdRP_B | CUGAGACGGUCGGGUCGGCGGUUCA (SEQ ID NO: 78) |
| broc_rot_arb_b10_ | GGGUAUAGAUUAGCUAAUGAGUGUGCUCAAGUAUUGAGCCUCCAUA |
| covid19rev_ | CUCCAUAGUUGAGCACACUCUCGAGUAGAGUGUGGGCUCAGAUUCG |
| RdRP_A | UCUGAGACGGUCGGGUCGAGUGUGCUC (SEQ ID NO: 79) |
| broc_rot_arb_b10_ | GGGAUUGAGUGAAAUGGUCAUGUGUGGCGGUUCACUAUGGGACGAA |
| covid19rev_ | AUACUGAUCCGCCACACAUGUCGAGUAGAGUGUGGGCUCAGAUUCG |
| RdRP_B | UCUGAGACGGUCGGGUCCAUGUGUGGC (SEQ ID NO: 80) |

TABLE 5
Sequences of the Corn RNA aptasensors tested in FIG. 7

| Name | Sequence |
|---|---|
| corn_b35_COVID- | GGGCAUGUGCAUUACCAUGGACUUGACAAUACAGAUCACAAUACA |
| 19_ORF1b_sensA | CUGAACUGAAUUGUCAAGUCCGAGGAAGGAGGUCUGAGGAGGUCA |
| | CUGGACUUGACAAUAAC (SEQ ID NO: 81) |
| corn_b35_COVID- | GGGAUACAGAUCAUGGUUGCUUUGUAGGUUACCUGUAAGACAUG |
| 19_ORF1b_sensB | AAUUAGAGGCAACCUACAAAGCGAGGAAGGAGGUCUGAGGAGGUC |
| | ACUGCUUUGUAGGUUAGA (SEQ ID NO: 82) |
| corn_b35_COVID- | GGGACUUGACAAUACAGAUCAUGGUUGCUUUGUAGGAACAAUAAC |
| 19_ORF1b_sensC | CUUCAACGCAACCAUGAUCGAGGAAGGAGGUCUGAGGAGGUCACU |
| | GAUCAUGGUUGCAAC (SEQ ID NO: 83) |
| corn_b35_COVID- | GGGCAACUAGCUACAUGUGCAUUACCAUGGACUUGACAAGAAUAA |
| 19_ORF1b_sensD | GUGUAAAGACCAUGGUAAUGCGAGGAAGGAGGUCUGAGGAGGUC |
| | ACUGCAUUACCAUGGAAC (SEQ ID NO: 84) |
| corn_b35_COVID- | GGGCACAACUAGCUACAUGUGCAUUACCAUGGACUUGAACACUAA |
| 19_ORF1b_sensE | AUCACGUCAAUGGUAAUGCACGAGGAAGGAGGUCUGAGGAGGUCA |
| | CUGUGCAUUACCAUCUC (SEQ ID NO: 85) |
| corn_b35_COVID- | GGGAUUGCAUCACAACUAGCUACAUGUGCAUUACCAUGCACACUC |
| 19_ORF1b_sensF | ACAUCGUACUGCACAUGUAGCGAGGAAGGAGGUCUGAGGAGGUCA |
| | CUGCUACAUGUGCAUCC (SEQ ID NO: 86) |

TABLE 6
Sequences of the Corn RNA aptasensors tested in FIG. 8

| Name | Sequence |
|---|---|
| corn_b35_COVID- | GGGUUUAACAUAUAGUGAACCGCCACACAUGACCAUGAAACGAAA |
| 19_RdRP_sensA | UGAUCAAGUGUGGCGGUUCGAGGAAGGAGGUCUGAGGAGGUCACU |
| | GAACCGCCACACGGC (SEQ ID NO: 87) |
| corn_b35_COVID- | GGGUUAGCAUAAGCAGUUGUGGCAUCUCCUGAUGAGGUAAAGCAA |
| 19_RdRP_sensB | GACCGCAUCAGGAGAUGCCACGAGGAAGGAGGUCUGAGGAGGUCA |
| | CUGUGGCAUCUCCUAAG (SEQ ID NO: 88) |
| corn_b35_COVID- | GGGAUGAGGUUCCACCUGGUUUAACAUAUAGUGAACCGAACUUUA |
| 19_RdRP_sensC | ACGGAUCAGUAUAUGUUAAACGAGGAAGGAGGUCUGAGGAGGUCA |
| | CUGUUUAACAUAUACCA (SEQ ID NO: 89) |
| corn_b35_COVID- | GGGCAUCUCCUGAUGAGGUUCCACCUGGUUUAACAUAAUCUAGCG |
| 19_RdRP_sensD | UAUCUUAUACCAGGUGGAACGAGGAAGGAGGUCUGAGGAGGUCAC |
| | UGUUCCACCUGGUUCC (SEQ ID NO: 90) |
| corn_b35_COVID- | GGGACACUAUUAGCAUAAGCAGUUGUGGCAUCUCCUGAGUUACAU |
| 19_RdRP_sensE | AUCACGAGUUGCCACAACUGCGAGGAAGGAGGUCUGAGGAGGUCA |
| | CUGCAGUUGUGGCAGUA (SEQ ID NO: 91) |
| corn_b35_COVID- | GGGUUGUGGCAUCUCCUGAUGAGGUUCCACCUGGUUUCAAAGUAC |
| 19_RdRP_sensF | AAAUCAGAUGGAACCUCAUCGAGGAAGGAGGUCUGAGGAGGUCAC |
| | UGAUGAGGUUCCAAGC (SEQ ID NO: 92) |

TABLE 7
Sequences of the Corn RNA aptasensors tested in FIG. 9

| Name | Sequence |
|---|---|
| 7.1 FIG. 9A | |
| corn_b35_COVID- | GGGAGUACGCACACAAUCGAAGCGCAGUAAGGAUGGCUAAAGUAAC |
| 19_E_sensA | AGCGAUCAUUACUGCGCUUCGAGGAAGGAGGUCUGAGGAGGUCACU |
| | GAAGCGCAGUAAACA (SEQ ID NO: 93) |
| corn_b35_COVID- | GGGAGCGCAGUAAGGAUGGCUAGUGUAACUAGCAAGAACGUAACAU |
| 19_E_sensB | UUCGUGCAAGUUACACUAGCGAGGAAGGAGGUCUGAGGAGGUCACU |
| | GCUAGUGUAACUGAA (SEQ ID NO: 94) |
| corn_b35_COVID- | GGGUAGUGUAACUAGCAAGAAUACCACGAAAGCAAGAACAUUAACA |
| 19_E_sensC | UUCCUGCCUUCGUGGUAUUCGAGGAAGGAGGUCUGAGGAGGUCACU |
| | GAAUACCACGAACAA (SEQ ID NO: 95) |
| corn_b35_COVID- | GGGCGAAAGCAAGAAAAAGAAGUACGCUAUUAACUAUUCAAUCUAC |
| 19_E_sensD | AAUCGUUCAUAGCGUACUUCGAGGAAGGAGGUCUGAGGAGGUCACU |
| | GAAGUACGCUAUACA (SEQ ID NO: 96) |
| corn_b35_COVID- | GGGAAGAAUACCACGAAAGCAAGAAAAAGAAGUACGCUAAGAACGA |
| 19_E_sensE | AGCCUACAUCUUUUUCUUGCGAGGAAGGAGGUCUGAGGAGGUCACU |
| | GCAAGAAAAAGACCA (SEQ ID NO: 97) |
| corn_b35_COVID- | GGGUAAGGAUGGCUAGUGUAACUAGCAAGAAUACCACGGAGUAAGG |
| 19_E_sensF | UGAUAUUCUUGCUAGUUACGAGGAAGGAGGUCUGAGGAGGUCACUG |
| | UAACUAGCAAGACA (SEQ ID NO: 98) |
| corn_b35_COVID- | GGGCACACAAUCGAAGCGCAGUAAGGAUGGCUAGUGUAGAUAAGAA |
| 19_E_sensG | CAAUAGACAUCCUUACUGCGAGGAAGGAGGUCUGAGGAGGUCACUG |
| | CAGUAAGGAUGAAA (SEQ ID NO: 99) |
| corn_b35_COVID- | GGGUAGCAAGAAUACCACGAAAGCAAGAAAAAGAAGUACGACACAU |
| 19_E_sensH | UACAUCUGUUUCUUGCUUUCGAGGAAGGAGGUCUGAGGAGGUCACU |
| | GAAAGCAAGAAACUA (SEQ ID NO: 100) |
| 7.2 FIG. 9B | |
| corn_b35_COVID- | GGGCAAUUUGCGGCCAAUGUUUGUAAUCAGUUCCUUGUAGAAUAGA |
| 19_N_sensA | ACACGGACCUGAUUACAAACGAGGAAGGAGGUCUGAGGAGGUCACU |
| | GUUUGUAAUCAGAUA (SEQ ID NO: 101) |
| corn_b35_COVID- | GGGAGGUGUGACUUCCAUGCCAAUGCGCGACAUUCCGAGUAACAUAU |
| 19_N_sensB | CGAAAUCUCGCGCAUUGGCGAGGAAGGAGGUCUGAGGAGGUCACUG |
| | CCAAUGCGCGACUA (SEQ ID NO: 102) |
| corn_b35_COVID- | GGGCUGGGGGCAAAUUGUGCAAUUUGCGGCCAAUGUUUCAUAUACU |
| 19_N_sensC | AAACAUUAGCCGCAAAUUGCGAGGAAGGAGGUCUGAGGAGGUCACU |
| | GCAAUUUGCGGCACA (SEQ ID NO: 103) |
| corn_b35_COVID- | GGGAAGAACGCUGAAGCGCUGGGGGCAAAUUGUGCAACAUGAAACU |
| 19_N_sensD | UGAACACUUUGCCCCCAGCGAGGAAGGAGGUCUGAGGAGGUCACUGC |
| | UGGGGGCAAAUGA (SEQ ID NO: 104) |
| corn_b35_COVID- | GGGCGCGACAUUCCGAAGAACGCUGAAGCGCUGGGGGCAUAUACACC |
| 19_N_sensE | CUCAGAGCUUCAGCGUUCGAGGAAGGAGGUCUGAGGAGGUCACUGA |
| | ACGCUGAAGCAAA (SEQ ID NO: 10) |
| corn_b35_COVID- | GGGCCGAAGAACGCUGAAGCGCUGGGGGCAAAUUGUGCAAGAUACC |
| 19_N_sensF | GCAGAAUAUGCCCCCAGCGCGAGGAAGGAGGUCUGAGGAGGUCACUG |
| | CGCUGGGGGCAGAA (SEQ ID NO: 105) |
| 7.3 FIG. 9C | |
| corn_b35_COVID- | GGGAUUGUUAGCAGGAUUGCGGGUGCCAAUGUGAUCUUAAAGACGG |
| 19_CDC_N3_sensA | AAGUUCAAAUUGGCACCCGCGAGGAAGGAGGUCUGAGGAGGUCACU |
| | GCGGGUGCCAAUAGA (SEQ ID NO: 106) |
| corn_b35_COVID- | GGGCAGCAUUGUUAGCAGGAUUGCGGGUGCCAAUGUGACAUAACAA |
| 19_CDC_N3_sensB | UCACAUUAGCACCCGCAAUCGAGGAAGGAGGUCUGAGGAGGUCACUG |
| | AUUGCGGGUGCGUC (SEQ ID NO: 107) |
| corn_b35_COVID- | GGGAGCAGGAUUGCGGGUGCCAAUGUGAUCUUUUGGUGAAACGGAA |
| 19_CDC_N3_sensC | CACUAAACGAUCACAUUGGCGAGGAAGGAGGUCUGAGGAGGUCACU |
| | GCCAAUGUGAUCAAA (SEQ ID NO: 108) |
| corn_b35_COVID- | GGGCACGAUUGCAGCAUUGUUAGCAGGAUUGCGGGUGCGACUUAAA |
| 19_CDC_N3_sensD | GCAACCGAAAUCCUGCUAACGAGGAAGGAGGUCUGAGGAGGUCACU |
| | GUUAGCAGGAUUCCA (SEQ ID NO: 109) |
| corn_b35_COVID- | GGGUGUAGCACGAUUGCAGCAUUGUUAGCAGGAUUGCGAGAAUGAA |
| 19_CDC_N3_sensE | CGCCAUCGUGCUAACAAUGCGAGGAAGGAGGUCUGAGGAGGUCACU |
| | GCAUUGUUAGCAGUA (SEQ ID NO: 110) |

TABLE 8
Sequences of the NASBA primers tested in FIG. 10 with the Broccoli RNA aptasensor
broc_rot_arb_b08_covid19_ORF1b_A (SEQ ID NO: 19)

| Name | Sequence |
|---|---|
| (1) NASBA primer pair 1 (optimal pair) | |
| NASBA_broc_rot_arb_b08_covid19_ORF1b_ | AATTCTAATACGACTCACTATAGGGAGAAGGG |
| A_0003_fwd | GTTTTACAGGTAACCTACA (SEQ ID NO: 257) |
| NASBA_broc_rot_arb_b08_covid19_ORF1b_ | ACAAGCCGCATTAATCTTCA (SEQ ID NO: 258) |
| A_0003_rev | |
| (2) NASBA primer pair 2 | |
| NASBA_broc_rot_arb_b08_covid19_ORF1b_ | AATTCTAATACGACTCACTATAGGGAGAAGGG |
| A_0003_fwd | GTTTTACAGGTAACCTACA (SEQ ID NO: 259) |
| NASBA_broc_rot_arb_b08_covid19_ORF1b_ | CTACAAGCCGCATTAATCTTCA (SEQ ID NO: 260) |
| A_0009_rev | |
| (3) NASBA primer pair 3 | |
| NASBA_broc_rot_arb_b08_covid19_ORF1b_ | AATTCTAATACGACTCACTATAGGGAGAAGGGT |
| A_0014_fwd | TTTACAGGTAACCTACA (SEQ ID NO: 261) |
| NASBA_broc_rot_arb_b08_covid19_ORF1b_ | ACAAGCCGCATTAATCTTCA (SEQ ID NO: 262) |
| A_0003_rev | |
| (4) NASBA primer pair 4 | |
| NASBA_broc_rot_arb_b08_covid19_ORF1b_ | AATTCTAATACGACTCACTATAGGGAGAAGGTG |
| A_0016_fwd | GGGTTTTACAGGTAACCTACA (SEQ ID NO: 263) |
| NASBA_broc_rot_arb_b08_covid19_ORF1b_ | ACAAGCCGCATTAATCTTCA (SEQ ID NO: 264) |
| A_0003_rev | |
| (5) NASBA primer pair 5 | |
| NASBA_broc_rot_arb_b08_covid19_ORF1b_ | AATTCTAATACGACTCACTATAGGGAGAAGGGT |
| A_014_fwd | TTTACAGGTAACCTACA (SEQ ID NO: 265) |
| NASBA_broc_rot_arb_b08_covid19_ORF1b_ | CTACAAGCCGCATTAATCTTCA (SEQ ID NO: 266) |
| A_0009_rev | |
| (6) NASBA primer pair 6 | |
| NASBA_broc_rot_arb_b08_covid19_ORF1b_ | AATTCTAATACGACTCACTATAGGGAGAAGGTG |
| A_0016_fwd | GGGTTTTACAGGTAACCTACA (SEQ ID NO: 267) |
| NASBA_broc_rot_arb_b08_covid19_ORF1b_ | CTACAAGCCGCATTAATCTTCA (SEQ ID NO: 268) |
| A_0009_rev | |
| (7) NASBA primer pair 7 | |
| NASBA_broc_rot_arb_b08_covid19_ORF1b_ | AATTCTAATACGACTCACTATAGGGAGAAGGCA |
| A_0028_fwd | ATGGGGTTTTACAGGTAACCTA (SEQ ID NO: 269) |
| NASBA_broc_rot_arb_b08_covid19_ORF1b_ | ACAAGCCGCATTAATCTTCA (SEQ ID NO: 270) |
| A_0003_rev | |
| (8) NASBA primer pair 8 | |
| NASBA_broc_rot_arb_b08_covid19_ORF1b_ | AATTCTAATACGACTCACTATAGGGAGAAGGAT |
| A_0029_fwd | GGGGTTTTACAGGTAACCTACA (SEQ ID NO: 271) |
| NASBA_broc_rot_arb_b08_covid19_ORF1b_ | ACAAGCCGCATTAATCTTCA (SEQ ID NO: 272) |
| A_0003_rev | |
| (9) NASBA primer pair 9 | |
| NASBA_broc_rot_arb_b08_covid19_ORF1b_ | AATTCTAATACGACTCACTATAGGGAGAAGGAC |
| A_0030_fwd | AATGGGGTTTTACAGGTA (SEQ ID NO: 273) |
| NASBA_broc_rot_arb_b08_covid19_ORF1b_ | ACAAGCCGCATTAATCTTCA (SEQ ID NO: 274) |
| A_0003_rev | |
| (10) NASBA primer pair 10 | |
| NASBA_broc_rot_arb_b08_covid19_ORF1b_ | AATTCTAATACGACTCACTATAGGGAGAAGGTT |
| A 0031 fwd | TTACAGGTAACCTACA (SEQ ID NO: 275) |
| NASBA_broc_rot_arb_b08_covid19_ORF1b_ | ACAAGCCGCATTAATCTTCA (SEQ ID NO: 276) |
| A_0003_rev | |

TABLE 9
Sequences of the NASBA primers tested in FIG. 11 with the Broccoli RNA aptasensor
broc_rot_arb_b08_covid19_RdRP_B (SEQ ID NO: 4)

| Name | Sequence |
|---|---|
| (1) NASBA primer pair 1 | |
| NASBA_broc_rot_arb_b08_covid19_ | AATTCTAATACGACTCACTATAGGGAGAAGGTC |
| RdRP_B_0163_fwd | ATGTGTGGCGGTTCACTATA (SEQ ID NO: 277) |
| NASBA_broc_rot_arb_b08_covid19_ | CTGTGTTGTAAATTGCGGACA (SEQ ID NO: 278) |
| RdRP_B_0163_rev | |
| (2) NASBA_primer pair 2 | |
| NASBA_broc_rot_arb_b08_covid19_ | AATTCTAATACGACTCACTATAGGGAGAAGGTC |
| RdRP_B_0163_fwd | ATGTGTGGCGGTTCACTATA (SEQ ID NO: 279) |
| NASBA_broc_rot_arb_b08_covid19_ | GTCTGTGTTGTAAATTGCGGACA (SEQ ID |
| RdRP_B_0165_rev | NO: 280) |
| (3) NASBA_primer pair 3 | |
| NASBA_broc_rot_arb_b08_covid19_ | AATTCTAATACGACTCACTATAGGGAGAAGGA |
| RdRP_B_0166_fwd | AATGGTCATGTGTGGCGGTTCA (SEQ ID NO: 281) |
| NASBA_broc_rot_arb_b08_covid19_ | GTCTGTGTTGTAAATTGCGGACA (SEQ ID |
| RdRP_B_0165_rev | NO: 282) |
| (4) NASBA_primer pair 4 | |
| NASBA_broc_rot_arb_b08_covid19_ | AATTCTAATACGACTCACTATAGGGAGAAGGTC |
| RdRP_B_0163_fwd | ATGTGTGGCGGTTCACTATA (SEQ ID NO: 283) |
| NASBA_broc_rot_arb_b08_covid19_ | TCTGTGTTGTAAATTGCGGACA (SEQ ID NO: 284) |
| RdRP_B_0167_rev | |
| (5) NASBA_primer pair 5 | |
| NASBA_broc_rot_arb_b08_covid19_ | AATTCTAATACGACTCACTATAGGGAGAAGGA |
| RdRP_B_0166_fwd | AATGGTCATGTGTGGCGGTTCA (SEQ ID NO: 285) |
| NASBA_broc_rot_arb_b08_covid19_ | CTGTGTTGTAAATTGCGGACA (SEQ ID NO: 286) |
| RdRP_B_0163_rev | |
| (6) NASBA_primer pair 6 | |
| NASBA_broc_rot_arb_b08_covid19_ | AATTCTAATACGACTCACTATAGGGAGAAGGTC |
| RdRP_B_0163_fwd | ATGTGTGGCGGTTCACTATA (SEQ ID NO: 287) |
| NASBA_broc_rot_arb_b08_covid19_ | TGTGTTGTAAATTGCGGACA (SEQ ID NO: 288) |
| RdRP_B_0169_rev | |
| (7) NASBA_primer pair 7 | |
| NASBA_broc_rot_arb_b08_covid19_ | AATTCTAATACGACTCACTATAGGGAGAAGGA |
| RdRP_B_0166_fwd | AATGGTCATGTGTGGCGGTTCA (SEQ ID NO: 289) |
| NASBA_broc_rot_arb_b08_covid19_ | AGTCTGTGTTGTAAATTGCGGACA (SEQ ID NO: 290) |
| RdRP_B_0171_rev | |
| (8) NASBA_primer pair 8 | |
| NASBA_broc_rot_arb_b08_covid19_ | AATTCTAATACGACTCACTATAGGGAGAAGGA |
| RdRP_B_0166_fwd | AATGGTCATGTGTGGCGGTTCA (SEQ ID NO: 291) |
| NASBA_broc_rot_arb_b08_covid19_ | TCTGTGTTGTAAATTGCGGACA (SEQ ID NO: 292) |
| RdRP_B_0167_rev | |
| (9) NASBA_primer pair 9 | |
| NASBA_broc_rot_arb_b08_covid19_ | AATTCTAATACGACTCACTATAGGGAGAAGGA |
| RdRP_B_0166_fwd | AATGGTCATGTGTGGCGGTTCA (SEQ ID NO: 293) |
| NASBA_broc_rot_arb_b08 covid19_ | TGTGTTGTAAATTGCGGACA (SEQ ID NO: 294) |
| RdRP_B_0169_rev | |
| (10) NASBA_primer pair 10 | |
| NASBA_broc_rot_arb_b08_covid19_ | AATTCTAATACGACTCACTATAGGGAGAAGGTG |
| RdRP_B_0176_fwd | GTCATGTGTGGCGGTTCACTA (SEQ ID NO: 295) |
| NASBA_broc_rot_arb_b08_covid19_ | GTCTGTGTTGTAAATTGCGGACA (SEQ ID |
| RdRP_B_0165_rev | NO: 296) |

TABLE 10
Sequences of the Broccoli RNA aptasensors for RT-RPA amplicons tested
in FIG. 12

| Name | Sequence |
|---|---|
| broc_rot_b07_2019_nCoV_ | GGGUGGAAGUCACACCUUCGGGAACGUGGUUGACCUACAG |
| N_antisense_targC_09 | UUACAAGUAUGUCUACCACGUUCCCGUCGAGUAGAGUGUG |
| | GGCUCAGAUUCGUCUGAGACGGUCGGGUCCGGGAAC (SEQ |
| | ID NO: 111) |
| broc_rot_b07_2019_nCoV_ | GGGCAGCGCUUCAGCGUUCUUCGGAAUGUCGCGCAUUGAG |
| N_antisense_targC_47 | AUGUAGCAACGCGAGACAUUCCGAAGUCGAGUAGAGUGUG |
| | GGCUCAGAUUCGUCUGAGACGGUCGGGUCCUUCGGA (SEQ |
| | ID NO: 112) |
| broc_rot_b08_2019_nCoV_ | GGGAUUGGCAUGGAAGUCACACCUUCGGGAACGUGGUUAA |
| N_antisense_targC_16 | ACGUCGAACGACGAUCCCGAAGGUGUUCGAGUAGAGUGUG |
| | GGCUCAGAUUCGUCUGAGACGGUCGGGUCACACCUUC |
| | (SEQ ID NO: 113) |
| broc_rot_b08_2019_nCoV_ | GGGAAUGUCGCGCAUUGGCAUGGAAGUCACACCUUCCCAG |
| N_antisense_targC_28 | UCAAGAAUGUGAGACUUCCAUGCCUCGAGUAGAGUGUGGG |
| | CUCAGAUUCGUCUGAGACGGUCGGGUCGGCAUGGA (SEQ |
| | ID NO: 114) |
| broc_rot_b09_2019_nCoV_ | GGGCAUUGGCAUGGAAGUCACACCUUCGGGAACGUGGCCU |
| N_antisense_targC_18 | UCGUUCCAGGUUGCCGAAGGUGUGAUCGAGUAGAGUGUGG |
| | GCUCAGAUUCGUCUGAGACGGUCGGGUCUCACACCUU |
| | (SEQ ID NO: 115) |
| broc_rot_b09_2019_nCoV_ | GGGAGCGUUCUUCGGAAUGUCGCGCAUUGGCAUGGAAGCU |
| N_antisense_targC_38 | UCGAUACUUGCAUACCAAUGCGCGACUCGAGUAGAGUGUG |
| | GGCUCAGAUUCGUCUGAGACGGUCGGGUCGUCGCGCAU |
| | (SEQ ID NO: 116) |
| broc_rot_b10_2019_nCoV_ | GGGAUUGGCAUGGAAGUCACACCUUCGGGAACGUGGUUAC |
| N_antisense_targC_16 | ACAUACAACGACGCUCCCGAAGGUGUUCGAGUAGAGUGUG |
| | GGCUCAGAUUCGUCUGAGACGGUCGGGUCACACCUUCGG |
| | (SEQ ID NO: 117) |
| broc_rot_b10_2019_nCoV_ | GGGAAUGUCGCGCAUUGGCAUGGAAGUCACACCUUCAACA |
| N_antisense_targC_28 | CGCAGAAUGUGAGACUUCCAUGCCUCGAGUAGAGUGUGGG |
| | CUCAGAUUCGUCUGAGACGGUCGGGUCGGCAUGGAAG |
| | (SEQ ID NO: 118) |

TABLE 11
Sequences of the Broccoli RNA aptasensors for detection of control
RNase P mRNAs tested in FIGS. 13-14

| Name | Sequence |
|---|---|
| broc_rot_arb_b09_ | GGGCGAGCGGGUUCUGACCUGAAGGCUCUGCGCGGACAGGAAUGA |
| covid19_RPrev_A | GUCGGCGUAGAGCCUUCAGGUCGAGUAGAGUGUGGGCUCAGAUUC |
| | GUCUGAGACGGUCGGGUCCCUGAAGGC (SEQ ID NO: 119) |
| Corn_b35_pc_RNaseP_ | GGGAGCGGGUUCUGACCUGAAGGCUCUGCGCGGACUUGAACGACG |
| antisens_E | ACAAAUCCACGCAGAGCCUUCGAGGAAGGAGGUCUGAGGAGGUCA |
| | CUGAAGGCUCUGCGAAG (SEQ ID NO: 120) |

TABLE 12
Sequences of the Broccoli RNA aptasensors for RT-LAMP amplicons and corresponding
LAMP primers tested in FIG. 15

| | | LAMP Primer Sequences | | | | | |
|---|---|---|---|---|---|---|---|
| Aptasensor | Aptasensor | LAMP | LAMP | LAMP | LAMP | LAMP | LAMP |
| Name | Sequence | F3 | B3 | FIP | BIP | LF | LB |
| broc_rot_b10_ | GGGAUAACCCUGUCCU | TCTTT | GTACC | CATGG | CTCTGG | GAAA | CTGTC |
| Ref2C_spike_ | ACCAUUUAAUGAUGGU | CACA | AAAA | AACCA | GACCAA | GGTA | CTACC |
| targ_Bloop_ | GUUUAGAAACGGAUAA | CGTG | ATCCA | AGTAA | TGGTAC | AGAA | ATTTA |
| antisense | UCACAAUCAUUAAAUG | GTGTT | GCCTC | CATTG | TAAGAG | CAAG | ATGAT |
| | GUCGAGUAGAGUGUGG | (SEQ | (SEQ | GAAAA | GACTTC | TCCTG | GGTGT |
| | GCUCAGAUUCGUCUGA | ID | ID | CCTGA | TCAGTG | AGT | (SEQ |
| | GACGGUCGGGUCCCAU | NO: 297) | NO: 298) | CAAAG | GAAGCA | (SEQ | ID |
| | UUAAUG (SEQ ID | | | TTTTCA | (SEQ ID | ID | NO: 302) |
| | NO: 121) | | | GATCC | NO: 300) | NO: 301) | |
| | | | | (SEQ ID | | | |
| | | | | NO: 299) | | | |
| broc_rot_b09_ | GGGUGAUAACCCUGUC | TCTTT | GTACC | CATGG | CTCTGG | GAAA | CTGTC |
| Ref2C_spike_ | CUACCAUUUAAUGAUG | CACA | AAAA | AACCA | GACCAA | GGTA | CTACC |
| targ_Bloop_ | GUGUUUAAUCACGCAA | CGTG | ATCCA | AGTAA | TGGTAC | AGAA | ATTTA |
| antisense | AGACCUUCAUUAAAUG | GTGTT | GCCTC | CATTG | TAAGAG | CAAG | ATGAT |
| | GUUCGAGUAGAGUGUG | (SEQ | (SEQ | GAAAA | GACTTC | TCCTG | GGTGT |
| | GGCUCAGAUUCGUCUG | ID | ID | CCTGA | TCAGTG | AGT | (SEQ |
| | AGACGGUCGGGUCACC | NO: 303) | NO: 304) | CAAAG | GAAGCA | (SEQ | ID |
| | AUUUAA (SEQ ID | | | TTTTCA | (SEQ ID | ID | NO: 308) |
| | NO: 122) | | | GATCC | NO: 306) | NO: 307) | |
| | | | | (SEQ ID | | | |
| | | | | NO: 305) | | | |
| broc_rot_b08_ | GGGUCUAAAGCCGAAA | AGTTT | TGAA | CAGGT | AGCAAG | GGCA | AACT |
| Ref15A_spike_ | AACCCUGAGGGAGAUC | GAGC | CCTCA | TGAAG | AAGAA | CCAA | GTTGG |
| targ_Floop_ | ACGGCCAUUAACGUAA | CATCA | ACAA | AGCAG | GATTGG | ATTCC | TCAAC |
| sense | UCGCCCUCAGGGUUUU | ACTCA | TTGTT | CAGAA | TTAGAT | AAAG | AAGA |
| | CGAGUAGAGUGUGGGC | (SEQ | TGA | GTGTA | GATGTC | GT | CGG |
| | UCAGAUUCGUCUGAGA | ID | (SEQ | CTGAA | TGATTG | (SEQ | (SEQ |
| | CGGUCGGGUCAAACCC | NO: 309) | ID | GATGA | TCCTCA | ID | ID |
| | UG (SEQ ID NO: 123) | | NO: 310) | TTACC | CTG | NO: 313) | NO: 314) |
| | | | | AAGG | (SEQ ID | | |
| | | | | (SEQ ID | NO: 312) | | |
| | | | | NO: 311) | | | |
| broc_rot_b09_ | GGGUCUAAAGCCGAAA | AGTTT | TGAA | CAGGT | AGCAAG | GGCA | AACT |
| Ref15A_spike_ | AACCCUGAGGGAGAUC | GAGC | CCTCA | TGAAG | AAGAA | CCAA | GTTGG |
| targ_Floop_ | ACGUUUGCAUUCGUCA | CATCA | ACAA | AGCAG | GATTGG | ATTCC | TCAAC |
| sense | UCACCCUCAGGGUUUU | ACTCA | TTGTT | CAGAA | TTAGAT | AAAG | AAGA |
| | CGAGUAGAGUGUGGGC | (SEQ | TGA | GTGTA | GATGTC | GT | CGG |
| | UCAGAUUCGUCUGAGA | ID | (SEQ | CTGAA | TGATTG | (SEQ | (SEQ |
| | CGGUCGGGUCAAACCC | NO: 315) | ID | GATGA | TCCTCA | ID | ID |
| | UGA (SEQ ID NO: 124) | | NO: 316) | TTACC | CTG | NO: 319) | NO: 320) |
| | | | | AAGG | (SEQ ID | | |
| | | | | (SEQ ID | NO: 318) | | |
| | | | | NO: 317) | | | |
| broc_rot_b08_ | GGGUCCAAUUAACACC | CCAG | CCGTC | AGCGG | AATTCC | TTATT | TTCCA |
| Ref5A_ | AAUAGCAGUCCAGAUG | AATG | ACCA | TGAAC | CTCGAG | GGGT | ATTAA |
| nucleocapsid_ | ACCAAAGCGCUCAAUU | GAGA | CCAC | CAAGA | GACAAG | AAAC | CACC |
| targ_Bloop_ | UCGUCGUCUGGACUGC | ACGC | GAATT | CGCAG | GCGAGC | CTTGG | AATA |
| antisense | UAUCGAGUAGAGUGUG | AGTG | (SEQ | GGCGC | TCTTCG | GGC | GCAG |
| | GGCUCAGAUUCGUCUG | (SEQ | NO: 322) | GATCA | GTAGTA | (SEQ | TCC |
| | AGACGGUCGGGUCUAG | ID | | AAACA | GCCAA | ID | (SEQ |
| | CAGUC (SEQ ID NO: | NO: 321) | | ACG | (SEQ ID | NO: 325) | ID |
| | 125) | | | (SEQ ID | NO: 324) | | NO: 326) |
| | | | | NO: 323) | | | |
| broc_rot_b07_ | GGGAUAGUCAACAAAC | AGTTT | TGAA | CAGGT | AGCAAG | GGCA | AACT |
| Ref15A_nsp3_ | UGUUGGUCAACAAGAC | GAGC | CCTCA | TGAAG | AAGAA | CCAA | GTTGG |
| targ_Bloop_ | GGCCACCAUAUGCCAU | CATCA | ACAA | AGCAG | GATTGG | ATTCC | TCAAC |
| antisense | CUAGUUGACCAACAGU | ACTCA | TTGTT | CAGAA | TTAGAT | AAAG | AAGA |
| | CGAGUAGAGUGUGGGC | (SEQ | TGA | GTGTA | GATGTC | GT | CGG |
| | UCAGAUUCGUCUGAGA | ID | (SEQ | CTGAA | TGATTG | (SEQ | (SEQ |
| | CGGUCGGGUCCUGUUG | NO: 327) | ID | GATGA | TCCTCA | ID | ID |
| | G (SEQ ID NO: 126) | | NO: 328) | TTACC | CTG | NO: 331) | NO: 332) |
| | | | | AAGG | (SEQ ID | | |
| | | | | (SEQ ID | NO: 330) | | |
| | | | | NO: 329) | | | |
| broc_rot_b08_ | GGGAUAGUCAACAAAC | AGTTT | TGAA | CAGGT | AGCAAG | GGCA | AACT |
| Ref15A_nsp3_ | UGUUGGUCAACAAGAC | GAGC | CCTCA | TGAAG | AAGAA | CCAA | GTTGG |
| targ_Bloop_ | GGCACAAGCAAGCCAU | CATCA | ACAA | AGCAG | GATTGG | ATTCC | TCAAC |
| antisense | CUAGUUGACCAACAGU | ACTCA | TTGTT | CAGAA | TTAGAT | AAAG | AAGA |
| | CGAGUAGAGUGUGGGC | (SEQ | TGA | GTGTA | GATGTC | GT | CGG |
| | UCAGAUUCGUCUGAGA | ID | (SEQ | CTGAA | TGATTG | (SEQ | (SEQ |
| | CGGUCGGGUCCUGUUG | NO: 333) | ID | GATGA | TCCTCA | ID | ID |
| | GU (SEQ ID NO: 127) | | NO: 3340 | TTACC | CTG | NO: 337) | NO: 338) |
| | | | | AAGG | (SEQ ID | | |
| | | | | (SEQ ID | NO: 336) | | |
| | | | | NO: 335) | | | |
| broc_rot_b07_ | GGGUCUAAAGCCGAAA | AGTTT | TGAA | CAGGT | AGCAAG | GGCA | AACT |
| Ref15A_spike_ | AACCCUGAGGGAGAUC | GAGC | CCTCA | TGAAG | AAGAA | CCAA | GTTGG |
| targ_Floop_ | ACGUUUUAUCUCGUUA | CATCA | ACAA | AGCAG | GATTGG | ATTCC | TCAAC |
| sense | UCGCCCUCAGGGUUUU | ACTCA | TTGTT | CAGAA | TTAGAT | AAAG | AAGA |
| | CGAGUAGAGUGUGGGC | (SEQ | TGA | GTGTA | GATGTC | GT | CGG |
| | UCAGAUUCGUCUGAGA | ID | (SEQ | CTGAA | TGATTG | (SEQ | (SEQ |
| | CGGUCGGGUCAAACCC | NO: 339) | ID | GATGA | TCCTCA | ID | ID |
| | U (SEQ ID NO: 128) | | NO: 340) | TTACC | CTG | NO: 343) | NO: 344) |
| | | | | AAGG | (SEQ ID | | |
| | | | | (SEQ ID | NO: 342) | | |
| | | | | NO: 341) | | | |
| broc_rot_b08_ | GGGUCGUUCCUCAUCA | AGAT | CCATT | TGCTC | GGCGGC | GCAA | GTTCC |
| Ref2B_ | CGUAGUCGCAACAGUU | CACAT | GCCA | CCTTCT | AGTCAA | TGTTG | TCATC |
| nucleocapsid_ | CAAGAACACGAAACUU | TGGC | GCCAT | GCGTA | GCCTCT | TTCCT | ACGT |
| targ_Bloop_ | CAUGAUCUGUUGCGAC | ACCC | TCTAG | GAAGC | TCCCTA | TGAG | AGTC |
| antisense | UAUCGAGUAGAGUGUG | G (SEQ | C (SEQ | CAATG | CTGCTG | GAAG | GCAA |
| | GGCUCAGAUUCGUCUG | ID | ID | CTGCA | CCTGGA | TT | CA |
| | AGACGGUCGGGUCUAG | NO: 345) | NO: 346) | ATCGT | GTT | (SEQ | (SEQ |
| | UCGCA (SEQ ID NO: | | | GCTAC | (SEQ ID | ID | ID |
| | 129) | | | (SEQ ID | NO: 348) | NO: 349) | NO: 350) |
| | | | | NO: 347) | | | |
| broc_rot_b09_ | GGGUCCAAUUAACACC | CCAG | CCGTC | AGCGG | AATTCC | TTATT | TTCCA |
| Ref5A_ | AAUAGCAGUCCAGAUG | AATG | ACCA | TGAAC | CTCGAG | GGGT | ATTAA |
| nucleocapsid_ | ACCAAAGCAGCUAAUU | GAGA | CCAC | CAAGA | GACAAG | AAAC | CACC |
| targ_Bloop_ | UCGUCGUCUGGACUGC | ACGC | GAATT | CGCAG | GCGAGC | CTTGG | AATA |
| antisense | UAUCGAGUAGAGUGUG | AGTG | (SEQ | GGCGC | TCTTCG | GGC | GCAG |
| | GGCUCAGAUUCGUCUG | (SEQ | ID | GATCA | GTAGTA | (SEQ | TCC |
| | AGACGGUCGGGUCUAG | ID | NO: 352) | AAACA | GCCAA | NO: 355) | (SEQ |
| | CAGUCC (SEQ ID | NO: 351) | | ACG | (SEQ ID | | ID |
| | NO: 130) | | | (SEQ ID | NO: 354) | | NO: 356) |
| | | | | NO: 353) | | | |
| broc_rot_b10_ | GGGAGGUUUACCCAAU | TGGA | GCCTT | CCACT | CGCGAT | TGAAT | GGTTT |
| Ref2A_ | AAUACUGCGUCUUGGU | CCCCA | GTCCT | GCGTT | CAAAAC | CTGA | ACCC |
| nucleocapsid_ | UCACCGUUUAGGUUCG | AAAT | CGAG | CTCCA | AACGTC | GGGT | AATA |
| targ_Bloop_ | GCGAAACAAGACGCAG | CAGC | GGAA | TTCTG | GGCCCT | CCACC | ATACT |
| antisense | UAUCGAGUAGAGUGUG | G (SEQ | T (SEQ | GTAAA | TGCCAT | AAA | GCGTC |
| | GGCUCAGAUUCGUCUG | ID | NO: 358) | TGCAC | GTTGAG | (SEQ | TT |
| | AGACGGUCGGGUCUAC | NO: 357) | | CCCGC | TGAGA | ID | (SEQ |
| | UGCGUCU (SEQ ID | | | ATTAC | (SEQ ID | NO: 361) | ID |
| | NO: 131) | | | G (SEQ | NO: 360) | | NO: 362) |
| | | | | ID | | | |
| | | | | NO: 359) | | | |
| broc_rot_b08_ | GGGAGGCCAUAAUUCU | AGTTT | TGAA | CAGGT | AGCAAG | GGCA | AACT |
| Ref15A_RdRP_ | AAGCAUGUUAGGCAUG | GAGC | CCTCA | TGAAG | AAGAA | CCAA | GTTGG |
| targ_Floop_ | GCUGCCAUAUAAGCGA | CATCA | ACAA | AGCAG | GATTGG | ATTCC | TCAAC |
| sense | UGACUAACAUGCUUAU | ACTCA | TTGTT | CAGAA | TTAGAT | AAAG | AAGA |
| | CGAGUAGAGUGUGGGC | (SEQ | TGA | GTGTA | GATGTC | GT | CGG |
| | UCAGAUUCGUCUGAGA | ID | (SEQ | CTGAA | TGATTG | (SEQ | (SEQ |
| | CGGUCGGGUCUAAGCA | NO: 363) | ID | GATGA | TCCTCA | ID | ID |
| | UG (SEQ ID NO: 132) | | NO: 364) | TTACC | CTG | NO: 367) | NO: 368) |
| | | | | AAGG | (SEQ ID | | |
| | | | | (SEQ ID | NO: 366) | | |
| | | | | NO: 365) | | | |
| broc_rot_b07 | GGGAGGUUUACCCAAU | TGGA | GCCTT | CCACT | CGCGAT | TGAAT | GGTTT |
| Ref2A_ | AAUACUGCGUCUUGGU | CCCCA | GTCCT | GCGTT | CAAAAC | CTGA | ACCC |
| nucleocapsid_ | UCACCGGGAUCGGACG | AAAT | CGAG | CTCCA | AACGTC | GGGT | AATA |
| targ_Bloop_ | GCGAAGCAAGACGCAG | CAGC | GGAA | TTCTG | GGCCCT | CCACC | ATACT |
| antisense | UAUCGAGUAGAGUGUG | G (SEQ | T (SEQ | GTAAA | TGCCAT | AAA | GCGTC |
| | GGCUCAGAUUCGUCUG | ID | ID | TGCAC | GTTGAG | (SEQ | TT |
| | AGACGGUCGGGUCUAC | NO: 369) | NO: 370) | CCCGC | TGAGA | ID | (SEQ |
| | UGCG (SEQ ID | | | ATTAC | (SEQ ID | NO: 373) | ID |
| | NO: 133) | | | G (SEQ | NO: 372) | | NO: 374) |
| | | | | ID | | | |
| | | | | NO: 371) | | | |
| broc_rot_b08_ | GGGAAGGUUUACCCAA | TGGA | GCCTT | CCACT | CGCGAT | TGAAT | GGTTT |
| Ref2A_ | UAAUACUGCGUCUUGG | CCCCA | GTCCT | GCGTT | CAAAAC | CTGA | ACCC |
| nucleocapsid_ | UUCACCGCUGGUUAGG | AAAT | CGAG | CTCCA | AACGTC | GGGT | AATA |
| targ_Bloop_ | UCAACGAAGACGCAGU | CAGC | GGAA | TTCTG | GGCCCT | CCACC | ATACT |
| antisense | AUUCGAGUAGAGUGUG | G (SEQ | T (SEQ | GTAAA | TGCCAT | AAA | GCGTC |
| | GGCUCAGAUUCGUCUG | ID | ID | TGCAC | GTTGAG | (SEQ | TT |
| | AGACGGUCGGGUCAUA | NO: 375) | NO: 376) | CCCGC | TGAGA | ID | (SEQ |
| | CUGCG (SEQ ID NO: | | | ATTAC | (SEQ ID | NO: 379) | ID |
| | 134) | | | G (SEQ | NO: 378) | | NO: 380) |
| | | | | ID | | | |
| | | | | NO: 377) | | | |
| broc_rot_b10 | GGGUCUAAAGCCGAAA | AGTTT | TGAA | CAGGT | AGCAAG | GGCA | AACT |
| Ref15A_spike_ | AACCCUGAGGGAGAUC | GAGC | CCTCA | TGAAG | AAGAA | CCAA | GTTGG |
| targ_Floop_ | ACGCCAGCUUACGUCA | CATCA | ACAA | AGCAG | GATTGG | ATTCC | TCAAC |
| sense | UCGCCCUCAGGGUUUU | ACTCA | TTGTT | CAGAA | TTAGAT | AAAG | AAGA |
| | CGAGUAGAGUGUGGGC | (SEQ | TGA | GTGTA | GATGTC | O | CGG |
| | UCAGAUUCGUCUGAGA | ID | (SEQ | CTGAA | TGATTG | GT | (SEQ |
| | CGGUCGGGUCAAACCC | NO: 381) | ID | GATGA | TCCTCA | (SEQ | ID |
| | UGAG (SEQ ID | | NO: 382) | TTACC | CTG | ID | NO: 386) |
| | NO: 135) | | | AAGG | (SEQ ID | NO: 385) | |
| | | | | (SEQ ID | NO: 384) | | |
| | | | | NO: 383) | | | |
| broc_rot_b08_ | GGGAAAGGUAAGAACA | TCTTT | GTACC | CATGG | CTCTGG | GAAA | CTGTC |
| Ref2C_spike_ | AGUCCUGAGUUGAAUG | CACA | AAAA | AACCA | GACCAA | GGTA | CTACC |
| targ_Floop_ | UAAAACUUCACUUCGU | CGTG | ATCCA | AGTAA | TGGTAC | AGAA | ATTTA |
| sense | UAUACCUUCAACUCAG | GTGTT | GCCTC | CATTG | TAAGAG | CAAG | ATGAT |
| | GAUCGAGUAGAGUGUG | (SEQ | (SEQ | GAAAA | GACTTC | TCCTG | GGTGT |
| | GGCUCAGAUUCGUCUG | ID | ID | CCTGA | TCAGTG | AGT | (SEQ |
| | AGACGGUCGGGUCUCC | NO: 387) | NO: 388) | CAAAG | GAAGCA | (SEQ | ID |
| | UGAGU (SEQ ID NO: | | | TTTTCA | (SEQ ID | ID | NO: 392) |
| | 136) | | | GATCC | NO: 390) | NO: 391) | |
| | | | | (SEQ ID | | | |
| | | | | NO: 389) | | | |

TABLE 13
Sequences of the Broccoli RNA aptasensors for RT-LAMP
amplicons of control sequences and corresponding LAMP
primers tested in FIG. 16

| | | LAMP Primer Sequences | | | | | |
|---|---|---|---|---|---|---|---|
| Aptasensor | | LAMP | LAMP | LAMP | LAMP | LAMP | LAMP |
| Name | Aptasensor Sequence | F3 | B3 | FIP | BIP | LF | LB |
| broc_rot_b07_ | GGGCGAGAAGAUGACCCAG | AGT | AGC | GAGCCA | CTGAAC | TGTG | CGAG |
| Ref17A_ctl_ | AUCAUGUUUGAGACCAAUA | ACC | CTG | CACGCA | CCCAAG | GTGC | AAGA |
| ACTB_mRNA_ | CGACGGUAUCAUACAUGAU | CCA | GAT | GCTCAT | GCCAAC | CAGA | TGAC |
| targ_Bloop_ | CUGGGUCGAGUAGAGUGUG | TCG | AGC | TGTATC | CGGCTG | TTTT | CCAG |
| antisense | GGCUCAGAUUCGUCUGAGA | AGC | AAC | ACCAAC | GGGTGT | CTCC | ATCA |
| | CGGUCGGGUCCCCAGAU | ACG | GTA | TGGGAC | TGAAGG | A | TGT |
| | (SEQ ID NO: 137) | (SEQ | CA | GACA | TC | (SEQ | (SEQ |
| | | ID | (SEQ | (SEQ | (SEQ | ID | ID |
| | | NO: | ID | ID NO: | ID NO: | NO: | NO: |
| | | 393) | NO: | 395) | 396) | 397) | 398) |
| | | | 394) | | | | |
| broc_rot_b09_ | GGGCGAGAAGAUGACCCAG | AGT | AGC | GAGCCA | CTGAAC | TGTG | CGAG |
| Ref17A_ctl_ | AUCAUGUUUGAGACCAAAG | ACC | CTG | CACGCA | CCCAAG | GTGC | AAGA |
| ACTB_mRNA_ | CGCAGGUAUCAUACAUGAU | CCA | GAT | GCTCAT | GCCAAC | CAGA | TGAC |
| targ_Bloop_ | CUGGGUCGAGUAGAGUGUG | TCG | AGC | TGTATC | CGGCTG | TTTT | CCAG |
| antisense | GGCUCAGAUUCGUCUGAGA | AGC | AAC | ACCAAC | GGGTGT | CTCC | ATCA |
| | CGGUCGGGUCCCCAGAUCA | ACG | GTA | TGGGAC | TGAAGG | A | TGT |
| | (SEQ ID NO: 138) | (SEQ | CA | GACA | TC | (SEQ | (SEQ |
| | | ID | (SEQ | (SEQ | (SEQ | ID | ID |
| | | NO: | ID | ID NO: | ID NO: | NO: | NO: |
| | | 399) | NO: | 401) | 402) | 403) | 404) |
| | | | 400) | | | | |
| broc_rot_b10_ | GGGCGAGAAGAUGACCCAG | AGT | AGC | GAGCCA | CTGAAC | TGTG | CGAG |
| Ref17A_ctl_ | AUCAUGUUUGAGACCGGUU | ACC | CTG | CACGCA | CCCAAG | GTGC | AAGA |
| ACTB_mRNA_ | UAAGGGUGUCACACAUGAU | CCA | GAT | GCTCAT | GCCAAC | CAGA | TGAC |
| targ_Bloop_ | CUGGGUCGAGUAGAGUGUG | TCG | AGC | TGTATC | CGGCTG | TTTT | CCAG |
| antisense | GGCUCAGAUUCGUCUGAGA | AGC | AAC | ACCAAC | GGGTGT | CTCC | ATCA |
| | CGGUCGGGUCCCCAGAUCA | ACG | GTA | TGGGAC | TGAAGG | A | TGT |
| | U (SEQ ID NO: 139) | (SEQ | CA | GACA | TC | (SEQ | (SEQ |
| | | ID | (SEQ | (SEQ | (SEQ | ID | ID |
| | | NO: | ID | ID NO: | ID NO: | NO: | NO: |
| | | 405) | NO: | 407) | 408) | 409) | 410) |
| | | | 406) | | | | |
| broc_rot_b08_ | GGGCGAGAAGAUGACCCAG | AGT | AGC | GAGCCA | CTGAAC | TGTG | CGAG |
| Ref17A_ctl_ | AUCAUGUUUGAGACCGAGA | ACC | CTG | CACGCA | CCCAAG | GTGC | AAGA |
| ACTB_mRNA_ | CUGAGGUGUCACACAUGAU | CCA | GAT | GCTCAT | GCCAAC | CAGA | TGAC |
| targ_Bloop_ | CUGGGUCGAGUAGAGUGUG | TCG | AGC | TGTATC | CGGCTG | TTTT | CCAG |
| antisense | GGCUCAGAUUCGUCUGAGA | AGC | AAC | ACCAAC | GGGTGT | CTCC | ATCA |
| | CGGUCGGGUCCCCAGAUC | ACG | GTA | TGGGAC | TGAAGG | A | TGT |
| | (SEQ ID NO: 140) | (SEQ | CA | GACA | TC | (SEQ | (SEQ |
| | | ID | (SEQ | (SEQ | (SEQ | ID | ID |
| | | NO: | ID | ID NO: | ID NO: | NO: | NO: |
| | | 411) | NO: | 413) | 414) | 415) | 416) |
| | | | 412) | | | | |
| broc_rot_b09_ | GGGAGAAGGUGUGGUGCCA | AGT | AGC | GAGCCA | CTGAAC | TGTG | CGAG |
| Ref17A_ctl_ | ACUUUCGAGAUGUAGAAAA | ACC | CTG | CACGCA | CCCAAG | GTGC | AAGA |
| ACTB_mRNA_ | UCUGGCUCGAGUAGAGUGU | CCA | GAT | GCTCAT | GCCAAC | CAGA | TGAC |
| targ_Floop_ | GAUUUUCUCCAUGUCGUUA | TCG | AGC | TGTATC | CGGCTG | TTTT | CCAG |
| antisense | GGGCUCAGAUUCGUCUGAG | AGC | AAC | ACCAAC | GGGTGT | CTCC | ATCA |
| | ACGGUCGGGUCGCCAGAUU | ACG | GTA | TGGGAC | TGAAGG | A | TGT |
| | U (SEQ ID NO: 141) | (SEQ | CA | GACA | TC | (SEQ | (SEQ |
| | | ID | (SEQ | (SEQ | (SEQ | ID | ID |
| | | NO: | ID | ID NO: | ID NO: | NO: | NO: |
| | | 417) | NO: | 419) | 420) | 421) | 422) |
| | | | 418) | | | | |
| broc_rot_b08_ | GGGAGGUGAAAUUCUUGGA | GTT | CCT | TGGCCT | GGCATT | AGAA | ATTC |
| Ref16A_ctl_ | CCGGCGCAAGACGGACUCG | CAA | CCG | CAGTTC | CGTATT | CCGC | CTTG |
| 18S_rRNA_ | UUUCUGUCAGUCGUGCGCC | AGC | ACT | CGAAAA | GCGCCG | GGTC | GACC |
| targ_Bloop_ | GGUCCAUCGAGUAGAGUGU | AGG | TTC | CCAACC | CTGGCA | CTAT | GGCG |
| antisense | GGGCUCAGAUUCGUCUGAG | CCC | GTT | TGGATA | AATGCT | TCCA | CAAG |
| | ACGGUCGGGUCUGGACCGG | GAG | CTT | CCGCAG | TTCGCT | TTAT | (SEQ |
| | (SEQ ID NO: 142) | (SEQ | GA | CTAGG | CTG | T | ID |
| | | ID | (SEQ | (SEQ | (SEQ | (SEQ | NO: |
| | | NO: | ID | ID NO: | ID NO: | ID | 428) |
| | | 423) | NO: | 425) | 426) | NO: | |
| | | | 424) | | | 427) | |

TABLE 14
Sequences of the Corn RNA aptasensors for RT-LAMP
amplicons of control sequences and
corresponding LAMP primers tested in FIG. 17

| | | LAMP Primer Sequences | | | | | |
|---|---|---|---|---|---|---|---|
| Aptasensor | Aptasensor | LAMP | LAMP | LAMP | LAMP | LAMP | LAMP |
| Name | Sequence | F3 | B3 | FIP | BIP | LF | LB |
| Corn_sta_ | GGGCGAGAAGAUGACCC | AG | AGC | GAGCCA | CTGAAC | TGTG | CGA |
| arb_b11_ | AGAUCAUGUUUGAGACC | TA | CTG | CACGCA | CCCAAG | GTGC | GAA |
| Ref17A_ | UUCACAUAACACUGACG | CC | GAT | GCTCAT | GCCAAC | CAGA | GAT |
| ctl_ACTB_ | GUAUCAAACAUGAUCGA | CC | AGC | TGTATC | CGGCTG | TTTT | GAC |
| mRNA_targ_ | GGAAGGAGGUCUGAGGA | AT | AAC | ACCAAC | GGGTGT | CTCC | CCA |
| Bloop_ | GGUCACUGAUCAUGUUU | CG | GTA | TGGGAC | TGAAG | A | GAT |
| antisense | GACA (SEQ ID NO: | AG | CA | GACA | GTC | (SEQ | CAT |
| | 143) | CA | (SEQ | (SEQ | (SEQ | ID | GT |
| | | CG | ID | ID NO: | ID NO: | NO: | (SEQ |
| | | (SEQ | NO: | 431) | 432) | 433) | ID |
| | | ID | 430) | | | | NO: |
| | | NO: | | | | | 434) |
| | | 429) | | | | | |
| Corn_sta_ | GGGAGAAGGUGUGGUGC | AG | AGC | GAGCCA | CTGAAC | TGTG | CGA |
| arb_b10_ | CAGAUUUUCUCCAUGUC | TA | CTG | CACGCA | CCCAAG | GTGC | GAA |
| Ref17A_ | GCCGAAUAACGACAUGU | CC | GAT | GCTCAT | GCCAAC | CAGA | GAT |
| ctl_ACTB_ | AGAAAAUCUGGCGAGGA | CC | AGC | TGTATC | CGGCTG | TTTT | GAC |
| mRNA_targ_ | AGGAGGUCUGAGGAGGU | AT | AAC | ACCAAC | GGGTGT | CTCC | CCA |
| Floop_ | CACUGCCAGAUUUUGAU | CG | GTA | TGGGAC | TGAAG | A | GAT |
| sense | (SEQ ID NO: 144) | AG | CA | GACA | GTC | (SEQ | CAT |
| | | CA | (SEQ | (SEQ | (SEQ | ID | GT |
| | | CG | ID | ID NO: | ID NO: | NO: | (SEQ |
| | | (SEQ | NO: | 437) | 438) | 439) | ID |
| | | ID | 436) | | | | NO: |
| | | NO: | | | | | 440) |
| | | 435 | | | | | |
| Corn_sta_ | GGGCGAGAAGAUGACCC | AG | AGC | GAGCCA | CTGAAC | TGTG | CGA |
| arb_b10_ | AGAUCAUGUUUGAGACC | TA | CTG | CACGCA | CCCAAG | GTGC | GAA |
| Ref17A_ | UUCAUAUACAACUGACG | CC | GAT | GCTCAT | GCCAAC | CAGA | GAT |
| ctl_ACTB_ | GUAUCAAACAUGAUCGA | CC | AGC | TGTATC | CGGCTG | TTTT | GAC |
| mRNA_targ_ | GGAAGGAGGUCUGAGGA | AT | AAC | ACCAAC | GGGTGT | CTCC | CCA |
| Bloop_ | GGUCACUGAUCAUGUUU | CG | GTA | TGGGAC | TGAAG | A | GAT |
| antisense | ACA (SEQ ID NO: | AG | CA | GACA | GTC | (SEQ | CAT |
| | 145) | CA | (SEQ | (SEQ | (SEQ | ID | GT |
| | | CG | ID | ID NO: | ID NO: | NO: | (SEQ |
| | | (SEQ | NO: | 443) | 444) | 445) | ID |
| | | ID | 442) | | | | NO: |
| | | 441) | | | | | 446) |
| Corn_sta_ | GGGAGAGGUGAAAUUCU | GT | CCT | TGGCCT | GGCATT | AGAA | ATT |
| arb_b09_ | UGGACCGGCGCAAGACG | TC | CCG | CAGTTC | CGTATT | CCGC | CCT |
| Ref16A_ | GACCAUCUAAACGGUAC | AA | ACT | CGAAAA | GCGCCG | GGTC | TGG |
| ctl_18S_ | GUAUUGCGCCGGUCCGA | AG | TTC | CCAACC | CTGGCA | CTAT | ACC |
| rRNA_targ_ | GGAAGGAGGUCUGAGGA | CA | GTT | TGGATA | AATGCT | TCCA | GGC |
| Bloop_ | GGUCACUGGACCGGCGU | GG | CTT | CCGCAG | TTCGCT | TTAT | GCA |
| antisense | UC (SEQ ID NO: | CC | GA | CTAGG | CTG | T | AG |
| | 146) | CG | (SEQ | (SEQ | (SEQ | (SEQ | (SEQ |
| | | AG | ID | ID NO: | ID NO: | ID | ID |
| | | (SEQ | NO: | 449) | 450) | NO: | NO: |
| | | ID | 448) | | | 451) | 452) |
| | | NO: | | | | | |
| | | 447) | | | | | |
| Corn_sta_ | GGGCGAGAAGAUGACCC | AG | AGC | GAGCCA | CTGAAC | TGTG | CGA |
| arb_b09_ | AGAUCAUGUUUGAGACC | TA | CTG | CACGCA | CCCAAG | GTGC | GAA |
| Ref17A_ | UUCAUAUGAUGCUGACG | CC | GAT | GCTCAT | GCCAAC | CAGA | GAT |
| ctl_ACTB_ | GUAUCAAACAUGAUCGA | CC | AGC | TGTATC | CGGCTG | TTTT | GAC |
| mRNA_targ_ | GGAAGGAGGUCUGAGGA | AT | AAC | ACCAAC | GGGTGT | CTCC | CCA |
| Bloop_ | GGUCACUGAUCAUGUUU | CG | GTA | TGGGAC | TGAAG | A | GAT |
| antisense | CG (SEQ ID NO: | AG | CA | GACA | GTC | (SEQ | CAT |
| | 147) | CA | (SEQ | (SEQ | (SEQ | ID | GT |
| | | CG | ID | ID NO: | ID NO: | NO: | (SEQ |
| | | (SEQ | NO: | 455) | 456) | 457) | ID |
| | | ID | 454) | | | | NO: |
| | | NO: | | | | | 458) |
| | | 453) | | | | | |
| Corn_sta_ | GGGCGAGAAGAUGACCC | AG | AGC | GAGCCA | CTGAAC | TGTG | CGA |
| arb_b12_ | AGAUCAUGUUUGAGACC | TA | CTG | CACGCA | CCCAAG | GTGC | GAA |
| Ref17A_ | UUCAACCUAAUCUGACG | CC | GAT | GCTCAT | GCCAAC | CAGA | GAT |
| ctl_ACTB_ | GUAUCAAACAUGAUCGA | CC | AGC | TGTATC | CGGCTG | TTTT | GAC |
| mRNA_targ_ | GGAAGGAGGUCUGAGGA | AT | AAC | ACCAAC | GGGTGT | CTCC | CCA |
| Bloop_ | GGUCACUGAUCAUGUUU | CG | GTA | TGGGAC | TGAAG | A | GAT |
| antisense | GAGUA (SEQ ID NO: | AG | CA | GACA | GTC | (SEQ | CAT |
| | 148) | CA | (SEQ | (SEQ | (SEQ | ID | GT |
| | | CG | ID | ID NO: | ID NO: | NO: | (SEQ |
| | | (SEQ | NO: | 461) | 462) | 463) | ID |
| | | ID | 460) | | | | NO: |
| | | NO: | | | | | 464) |
| | | 459) | | | | | |
| Corn_sta_ | GGGAGAAGGUGUGGUGC | AG | AGC | GAGCCA | CTGAAC | TGTG | CGA |
| arb_b09_ | CAGAUUUUCUCCAUGUC | TA | CTG | CACGCA | CCCAAG | GTGC | GAA |
| Ref17A_ | GGACAAGAACGAAAUGU | CC | GAT | GCTCAT | GCCAAC | CAGA | GAT |
| ctl_ACTB_ | AGAAAAUCUGGCGAGGA | CC | AGC | TGTATC | CGGCTG | TTTT | GAC |
| mRNA_targ_ | AGGAGGUCUGAGGAGGU | AT | AAC | ACCAAC | GGGTGT | CTCC | CCA |
| Floop_ | CACUGCCAGAUUUAGA | CG | GTA | TGGGAC | TGAAG | A | GAT |
| sense | (SEQ ID NO: | AG | CA | GACA | GTC | (SEQ | CAT |
| | 149) | CA | (SEQ | (SEQ | (SEQ | ID | GT |
| | | CG | ID | ID NO: | ID NO: | NO: | (SEQ |
| | | (SEQ | NO: | 467) | 468) | 469) | ID |
| | | ID | 466) | | | | NO: |
| | | NO: | | | | | 470) |
| | | 465) | | | | | |
| Corn_sta_ | GGGAGAGGUGAAAUUCU | GT | CCT | TGGCCT | GGCATT | AGAA | ATT |
| arb_b11_ | UGGACCGGCGCAAGACG | TC | CCG | CAGTTC | CGTATT | CCGC | CCT |
| Ref16A_ | GACCACUAUCACGGUAC | AA | ACT | CGAAAA | GCGCCG | GGTC | TGG |
| ctl_18S_ | GUAUUGCGCCGGUCCGA | AG | TTC | CCAACC | CTGGCA | CTAT | ACC |
| rRNA_targ_ | GGAAGGAGGUCUGAGGA | CA | GTT | TGGATA | AATGCT | TCCA | GGC |
| Bloop_ | GGUCACUGGACCGGCGC | GG | CTT | CCGCAG | TTCGCT | TTAT | GCA |
| antisense | ACAU (SEQ ID NO: | CC | GA | CTAGG | CTG | T | AG |
| | 150) | CG | (SEQ | (SEQ | (SEQ | (SEQ | (SEQ |
| | | AG | ID | ID NO: | ID NO: | ID | ID |
| | | (SEQ | NO: | 473) | 474) | NO: | NO: |
| | | ID | 472) | | | 475) | 476) |
| | | NO: | | | | | |
| | | 471) | | | | | |

TABLE 15
Sequences of the improved Red Broccoli RNA aptasensors
tested in FIG. 20

| Name | Sequence |
|---|---|
| std_red_broc_gen3_ | GGGUUUGGCAAUGUUGUUCCUUGAGGAAGUUGUAGCAACCUAC |
| b13_a15_Ref2B_nucleo- | UGCUACAACUUCCUCAAGGCGGUCGGGUCCGCAGAUAGAUCUC |
| capsid_targ_Floop_ | UAUCUGCGUUGAGUAGUGUGUGGCCUUGAGGAAGUU (SEQ |
| sense_A | ID NO: 151) |
| std_red_broc_gen3_ | GGGCUUUUGGCAAUGUUGUUCCUUGAGGAAGUUGUAGCAGAAC |
| b13_a17_Ref2B_nucleo- | GAUGCUACAACUUCCUCAAGGCGGUCGGGUCCCACAGAAUGGG |
| capsid_targ_Floop_ | CAUUCUGUGGUUGAGUAGUGUGUGGCCUUGAGGAAGUU (SEQ |
| sense_A | ID NO: 152) |
| std_red_broc_gen3_ | GGGUUUUGGCAAUGUUGUUCCUUGAGGAAGUUGUAGCAAUUAG |
| b14_a15_Ref2B_nucleo- | CUGCUACAACUUCCUCAAGGACGGUCGGGUCCCUCUAAGCGUU |
| capsid_targ_Floop_ | AGCUUAGAGGUUGAGUAGUGUGUGGUCCUUGAGGAAGUU |
| sense_A | (SEQ ID NO: 153) |
| std_red_broc_gen3_ | GGGCUUUUGGCAAUGUUGUUCCUUGAGGAAGUUGUAGCACGGU |
| b14_a18_Ref2B_nucleo- | GGCACGUGCUACAACUUCCUCAAGCGGUCGGGUCCCUGCCAGC |
| capsid_targ_Floop_ | UGCGGCUGGCAGGUUGAGUAGUGUGUGGCUUGAGGAAGUUGU |
| sense_A | (SEQ ID NO: 154) |
| std_red_broc_gen3_ | GGGUUUUGGCAAUGUUGUUCCUUGAGGAAGUUGUAGCACGAAC |
| b15_a17_Ref2B_nucleo- | GACAUCGUGCUACAACUUCCUCAAGCGGUCGGGUCCCUCAGAU |
| capsid_targ_Floop_ | GUGCGCAUCUGAGGUUGAGUAGUGUGUGGCUUGAGGAAGUUGU |
| sense_A | A (SEQ ID NO: 155) |
| std_red_broc_gen3_ | GGGCUUUUGGCAAUGUUGUUCCUUGAGGAAGUUGUAGCACGAG |
| b15_a17_Ref2B_nucleo- | AAGGCGUGCUACAACUUCCUCAAGGCGGUCGGGUCCCUUGCUU |
| capsid_targ_Floop_ | CGAUGGAAGCAAGGUUGAGUAGUGUGUGGCCUUGAGGAAGUUG |
| sense_B | U (SEQ ID NO: 156) |
| rot_red_broc_gen3_ | GGGAAUGUUGUUCCUUGAGGAAGUUGUAGCACGAUGGCUAGAU |
| b10_a16_Ref2B_nucleo- | CGUGCUACAACUUCGUUGAGUAGUGUGUGGCACAGAUGUGGGC |
| capsid_targ_Floop_ | AUCUGUGCGGUCGGGUCCGAAGUUGUAG (SEQ ID NO: |
| sense_A | 157) |
| rot_red_broc_gen3_ | GGGCUUUUGGCAAUGUUGUUCCUUGAGGAAGUUGUAGGCUGGA |
| b13_a15_Ref2B_nucleo- | CUACAACUUCCUCAAGGAAGUUGAGUAGUGUGUGGCAUCUCGC |
| capsid_targ_Floop_ | UAACGCGAGAUGCGGUCGGGUCCUUCCUUGAGGAAG (SEQ |
| sense_A | ID NO: 158) |
| rot_red_broc_gen3_ | GGGAUGUUGUUCCUUGAGGAAGUUGUAGCACGAUUGCAAUUGA |
| b13_a15_Ref2B_nucleo- | GCAAUCGUGCUACAACUUCGUUGAGUAGUGUGUGGCUGUGGUC |
| capsid_targ_Floop_ | CCGAGACCACAGCGGUCGGGUCCGAAGUUGUAGCAC (SEQ |
| sense_B | ID NO: 159) |
| rot_red_broc_gen3_ | GGGUUGGCAAUGUUGUUCCUUGAGGAAGUUGUAGCACGAUUGC |
| b14_a23_Ref2B_nucleo- | AGCGAACGAGCUGCAAUCGUGCUACAACUGUUGAGUAGUGUGU |
| capsid_targ_Floop_ | GGAUCGACUGGGCUCAGUCGAUCGGUCGGGUCCAGUUGUAGCA |
| sense_A | CGAU (SEQ ID NO: 160) |
| rot_red_broc_gen3_ | GGGUUUUGGCAAUGUUGUUCCUUGAGGAAGUUGUAGCACUUCC |
| b15_a15_Ref2B_nucleo- | CUGUGCUACAACUUCCUCAAGGAGUUGAGUAGUGUGUGGCGAG |
| capsid_targ_Floop_ | UUCGACCUCGAACUCGCGGUCGGGUCCUCCUUGAGGAAGUUG |
| sense_A | (SEQ ID NO: 161) |
| rot_red_broc_gen3_ | GGGUUUUGGCAAUGUUGUUCCUUGAGGAAGUUGUAGCACGAUU |
| b15_a23_Ref2B_nucleo- | GCAGAGUCGGCUGCAAUCGUGCUACAACUUCGUUGAGUAGUGU |
| capsid_targ_Floop_ | GUGGGAAUUCUCUCGAGAGAAUUCCGGUCGGGUCCGAAGUUGU |
| sense_A | AGCACGA (SEQ ID NO: 162) |
| std_red_broc_gen3_ | GGGUCAUCACGUAGUCGCAACAGUUCAAGAAAUUCAACUCCAG |
| b13_a30_Ref2B_nucleo- | GCAGCAGUAGUUGGAUACUGCUGCCUGGAGUUGACGGUCGGGU |
| capsid_targ_Bloop_ | CCGACUAUAGGUUCCUAUAGUCGUUGAGUAGUGUGUGGUCAAC |
| antisense_A | UCCAGGCA (SEQ ID NO: 163) |
| std_red_broc_gen3_ | GGGUCGCAACAGUUCAAGAAAUUCAACUCCAGGCAGCAGUAGA |
| b15_a17_Ref2B_nucleo- | CUGAUACUGCUGCCUGGAGUUGAAUCGGUCGGGUCCGGCUGUG |
| capsid_targ_Bloop_ | CCAAGGCACAGCCGUUGAGUAGUGUGUGGAUUCAACUCCAGGC |
| antisense_A | A (SEQ ID NO: 164) |
| std_red_broc_gen3_ | GGGUCACGUAGUCGCAACAGUUCAAGAAAUUCAACUCCAGGCA |
| b15_a25_Ref2B_nucleo- | GCAGUAGCUAGAUACUGCUGCCUGGAGUUGAAUCGGUCGGGUC |
| capsid_targ_Bloop_ | CGGUAGAGGGCAUCCUCUACCGUUGAGUAGUGUGUGGAUUCAA |
| antisense_A | CUCCAGGCA (SEQ ID NO: 165) |
| std_red_broc_gen3_ | GGGUCAUCACGUAGUCGCAACAGUUCAAGAAAUUCAACUCCAG |
| b15_a28_Ref2B_nucleo- | GCAGCAGUAUGAAGUUACUGCUGCCUGGAGUUGAAUCGGUCGG |
| capsid_targ_Bloop_ | GUCCGGAAUAACUCGGGUUAUUCCGUUGAGUAGUGUGUGGAUU |
| antisense_A | CAACUCCAGGCA (SEQ ID NO: 166) |
| std_red_broc_gen3_ | GGGUCCUCAUCACGUAGUCGCAACAGUUCAAGAAAUUCAACUC |
| b15_a33_Ref2B_nucleo- | CAGGCAGCAGUAGGACCUUACCUACUGCUGCCUGGAGUUGACG |
| capsid_targ_Bloop_ | GUCGGGUCCGGCAUCAGUCAGCUGAUGCCGUUGAGUAGUGUGU |
| antisense_A | GGUCAACUCCAGGCAGC (SEQ ID NO: 167) |
| std_red_broc_gen3_ | GGGUCGUUCCUCAUCACGUAGUCGCAACAGUUCAAGAAAUUCA |
| b15_a35_Ref2B_nucleo- | ACUCCAGGCAGCAGUAUACAUCUACUGCUGCCUGGAGUUGAAU |
| capsid_targ_Bloop_ | CGGUCGGGUCCUCGACUACAGGCGUAGUCGAGUUGAGUAGUGU |
| antisense_A | GUGGAUUCAACUCCAGGCA (SEQ ID NO: 168) |
| rot_red_broc_gen3_ | GGGUUCCUCAUCACGUAGUCGCAACAGUUCAAGAAGCUCAAUU |
| b12_a15_Ref2B_nucleo- | CUUGAACUGUUGCGACGUUGAGUAGUGUGUGGACUUGGUCUUG |
| capsid_targ_Bloop_ | AGACCAAGUCGGUCGGGUCCGUCGCAACAGUU (SEQ ID |
| antisense_A | NO: 169) |
| rot_red_broc_gen3_ | GGGCGUAGUCGCAACAGUUCAAGAAAUUCAACUCCAGGCAGCA |
| b12_a24_Ref2B_nucleo- | GUGAGUUAACUGCUGCCUGGAGUUGAGUUGAGUAGUGUGUGGA |
| capsid_targ_Bloop_ | UGGCAUCGAACGAUGCCAUCGGUCGGGUCCUCAACUCCAGGC |
| antisense_A | (SEQ ID NO: 170) |
| rot_red_broc_gen3_ | GGGCGUAGUCGCAACAGUUCAAGAAAUUCAACUCCAGGCAGCA |
| b13_a23_Ref2B_nucleo- | GUUGAUAUACUGCUGCCUGGAGUUGAAGUUGAGUAGUGUGUGG |
| capsid_targ_Bloop_ | AGGGUACGGGUCCGUACCCUCGGUCGGGUCCUUCAACUCCAGG |
| antisense_A | C (SEQ ID NO: 171) |
| rot_red_broc_gen3_ | GGGUCGUUCCUCAUCACGUAGUCGCAACAGUUCAAGAAAUUCA |
| b13_a36_Ref2B_nucleo- | ACUCCAGGCAGCAGUACUUGAACUGCUGCCUGGAGUUGAAGUU |
| capsid_targ_Bloop_ | GAGUAGUGUGUGGCCAGACGCAGCAGCGUCUGGCGGUCGGGUC |
| antisense_A | CUUCAACUCCAGGC (SEQ ID NO: 172) |
| rot_red_broc_gen3_ | GGGUAGUCGCAACAGUUCAAGAAAUUCAACUCCAGGCAGCAGU |
| b14_a21_Ref2B_nucleo- | AUCUGCACUGCUGCCUGGAGUUGAAUGUUGAGUAGUGUGUGGA |
| capsid_targ_Bloop_ | UAUAGGCAUGCGCCUAUAUCGGUCGGGUCCAUUCAACUCCAGG |
| antisense_A | C (SEQ ID NO: 173) |
| rot_red_broc_gen3_ | GGGCGUAGUCGCAACAGUUCAAGAAAUUCAACUCCAGGCAGCA |
| b14_a22_Ref2B_nucleo- | GUACCCGCACUGCUGCCUGGAGUUGAAUGUUGAGUAGUGUGUG |
| capsid_targ_Bloop_ | GAGUUCCGGGAAACCGGAACUCGGUCGGGUCCAUUCAACUCCA |
| antisense_A | GGC (SEQ ID NO: 174) |

TABLE 16
Sequences of the improved Orange Broccoli
RNA aptasensors tested in FIG. 21

| Name | Sequence |
|---|---|
| std_orange_broc_gen3_ | GGGUGAAUCUGAGGGUCCACCAAACGUAAUGCGGGGUGUCAC |
| b10_a18_Ref2A_nucleo- | AACCCCGCAUUACGUUUCGGUCGGGUCCGGGUAGAGCUAGCU |
| capsid_targ_Floop_ | CUACCCGUUGAGUAGCGUGUGGAAACGUAAUG (SEQ ID |
| sense_A | NO: 175) |
| std_orange_broc_gen3_ | GGGUGAAUCUGAGGGUCCACCAAACGUAAUGCGGGGUGGGUC |
| b10_a19_Ref2A_nucleo- | UACACCCCGCAUUACGUUCGGUCGGGUCCGGGGACAGCUUGC |
| capsid_targ_Floop_ | UGUCCCCGUUGAGUAGCGUGUGGAACGUAAUGC (SEQ ID |
| sense_A | NO: 176) |
| std_orange_broc_gen3_ | GGGUUGAAUCUGAGGGUCCACCAAACGUAAUGCGGGGUUAGC |
| b10_a19_Ref2A_nucleo- | CUACCCCGCAUUACGUUUCGGUCGGGUCCGAACCAUCUCGAG |
| capsid_targ_Floop_ | AUGGUUCGUUGAGUAGCGUGUGGAAACGUAAUG (SEQ ID |
| sense_B | NO: 177) |
| std_orange_broc_gen3_ | GGGUUGAAUCUGAGGGUCCACCAAACGUAAUGCGGGGUGUAC |
| b11_a19_Ref2A_nucleo- | UAACCCCGCAUUACGUUUGCGGUCGGGUCCGGCUCUAGGCAC |
| capsid_targ_Floop_ | CUAGAGCCGUUGAGUAGCGUGUGGCAAACGUAAUG (SEQ |
| sense_A | ID NO: 178) |
| std_orange_broc_gen3_ | GGGUACUGCCAGUUGAAUCUGAGGGUCCACCAAACGUGGAUG |
| b13_a15_Ref2A_nucleo- | AACGUUUGGUGGACCCUCAGCGGUCGGGUCCGAUCCUUGGUC |
| capsid_targ_Floop_ | ACAAGGAUCGUUGAGUAGCGUGUGGCUGAGGGUCCACC |
| sense_A | (SEQ ID NO: 179) |
| std_orange_broc_gen3_ | GGGUUGAAUCUGAGGGUCCACCAAACGUAAUGCGGGGUGCGU |
| b13_a19_Ref2A_nucleo- | UGAAGCACCCCGCAUUACGUUUGCGGUCGGGUCCGGGCUAAG |
| capsid_targ_Floop_ | CACGCUUAGCCCGUUGAGUAGCGUGUGGCAAACGUAAUGCG |
| sense_A | (SEQ ID NO: 180) |
| rot_orange_broc_gen3_ | GGGUGCCAGUUGAAUCUGAGGGUCCACCAAACGUAAUGCGAU |
| b13_a18_Ref2A_nucleo- | CUACCGCAUUACGUUUGGUGGACGUUGAGUAGCGUGUGGAAA |
| capsid_targ_Floop_ | UAGUGUGGGCACUAUUUCGGUCGGGUCCGUCCACCAAACGU |
| sense_A | (SEQ ID NO: 181) |
| rot_orange_broc_gen3_ | GGGCAGUUGAAUCUGAGGGUCCACCAAACGUAAUGCGGGGUC |
| b14_a17_Ref2A_nucleo- | UGAUCCCCGCAUUACGUUUGGUGGGUUGAGUAGCGUGUGGUA |
| capsid_targ_Floop_ | ACGAAGACAGCUUCGUUACGGUCGGGUCCCCACCAAACGUAA |
| sense_A | U (SEQ ID NO: 182) |
| rot_orange_broc_gen3_ | GGGUGCCAGUUGAAUCUGAGGGUCCACCAAACGUAAUGCGGA |
| b14_a17_Ref2A_nucleo- | GUAGCGCAUUACGUUUGGUGGACCGUUGAGUAGCGUGUGGUU |
| capsid_targ_Floop_ | AGGCGCUGUUGCGCCUAACGGUCGGGUCCGGUCCACCAAACG |
| sense_B | U (SEQ ID NO: 183) |
| rot_orange_broc_gen3_ | GGGCCAGUUGAAUCUGAGGGUCCACCAAACGUAAUGCGGAAU |
| b15_a15_Ref2A_nucleo- | GACGCAUUACGUUUGGUGGACCCGUUGAGUAGCGUGUGGAUU |
| capsid_targ_Floop_ | UAGAGACGGCUCUAAAUCGGUCGGGUCCGGGUCCACCAAACG |
| sense_A | U (SEQ ID NO: 184) |
| rot_orange_broc_gen3_ | GGGUACUGCCAGUUGAAUCUGAGGGUCCACCAAACGUAAUGC |
| b15_a19_Ref2A_nucleo- | GGUUCUACGCAUUACGUUUGGUGGACCCGUUGAGUAGCGUGU |
| capsid_targ_Floop_ | GGUCCUGAUACCCGUAUCAGGACGGUCGGGUCCGGGUCCACC |
| sense_A | AAACGU (SEQ ID NO: 185) |
| rot_orange_broc_gen3_ | GGGUGCCAGUUGAAUCUGAGGGUCCACCAAACGUAAUGCGGG |
| b15_a19_Ref2A_nucleo- | GAUCACACCCCGCAUUACGUUUGGUGGAGUUGAGUAGCGUGU |
| capsid_targ_Floop_ | GGUGAGGACGGGAACGUCCUCACGGUCGGGUCCUCCACCAAA |
| sense_B | CGUAAU (SEQ ID NO: 186) |
| std_orange_broc_gen3_ | GGGAAGGUUUACCCAAUAAUACUGCGUCUUGGUUCACUUACU |
| b11_a17_Ref2A_nucleo- | UGUGAACCAAGACGCAGUCGGUCGGGUCCGGCAACUGUAUGC |
| capsid_targ_Bloop_ | AGUUGCCGUUGAGUAGCGUGUGGACUGCGUCUUG (SEQ ID |
| antisense_A | NO: 187) |
| std_orange_broc_gen3_ | GGGAAGGUUUACCCAAUAAUACUGCGUCUUGGUUCACCGUCU |
| b11_a18_Ref2A_nucleo- | UAGGUGAACCAAGACGCAGCGGUCGGGUCCCAGUUGUCUCGA |
| capsid_targ_Bloop_ | GACAACUGGUUGAGUAGCGUGUGGCUGCGUCUUGG (SEQ |
| antisense_A | ID NO: 188) |
| std_orange_broc_gen3_ | GGGAUAAUACUGCGUCUUGGUUCACCGCUCUCACUCAACAAU |
| b14_a17_Ref2A_nucleo- | AAACUGUUGAGUGAGAGCGGUGAACGGUCGGGUCCGGGAUUU |
| capsid_targ_Bloop_ | CAUUAGAAAUCCCGUUGAGUAGCGUGUGGUUCACCGCUCUCA |
| antisense_A | C (SEQ ID NO: 189) |
| std_orange_broc_gen3_ | GGGAAGGUUUACCCAAUAAUACUGCGUCUUGGUUCACCGCUU |
| b14_a18_Ref2A_nucleo- | CCGCUAGCGGUGAACCAAGACGCAGCGGUCGGGUCCGGGUGA |
| capsid_targ_Bloop_ | CCUCAUGGUCACCCGUUGAGUAGCGUGUGGCUGCGUCUUGGU |
| antisense_A | UC (SEQ ID NO: 190) |
| std_orange_broc_gen3_ | GGGAUAAUACUGCGUCUUGGUUCACCGCUCUCACUCAACAUG |
| b15_a17_Ref2A_nucleo- | AUUAGAUGUUGAGUGAGAGCGGUGAACGGUCGGGUCCGCUGC |
| capsid_targ_Bloop_ | AUCUCACGAUGCAGCGUUGAGUAGCGUGUGGUUCACCGCUCU |
| antisense_A | CACU (SEQ ID NO: 191) |
| std_orange_broc_gen3_ | GGGAAGGUUUACCCAAUAAUACUGCGUCUUGGUUCACCGCUG |
| b15_a17_Ref2A_nucleo- | GUAAGAGCGGUGAACCAAGACGCAGUCGGUCGGGUCCUCCCG |
| capsid_targ_Bloop_ | UCGGCGACGACGGGAGUUGAGUAGCGUGUGGACUGCGUCUUG |
| antisense_B | GUUC (SEQ ID NO: 192) |
| rot_orange_broc_gen3_ | GGGAAUAAUACUGCGUCUUGGUUCACCGCUCUCACUCAUUAC |
| b12_a17_Ref2A_nucleo- | UUUGAGUGAGAGCGGUGAACGUUGAGUAGCGUGUGGAUGGGA |
| capsid_targ_Bloop_ | GCAAGGGCUCCCAUCGGUCGGGUCCGUUCACCGCUCU (SEQ |
| antisense_A | ID NO: 193) |
| rot_orange_broc_gen3_ | GGGAAUAAUACUGCGUCUUGGUUCACCGCUCUCACUCAUUGG |
| b14_a15_Ref2A_nucleo- | AUUGAGUGAGAGCGGUGAACCAGUUGAGUAGCGUGUGGAGCC |
| capsid_targ_Bloop_ | CGAGAUAGCUCGGGCUCGGUCGGGUCCUGGUUCACCGCUCU |
| antisense_A | (SEQ ID NO: 194) |
| rot_orange_broc_gen3_ | GGGAUAAUACUGCGUCUUGGUUCACCGCUCUCACUCAACGAC |
| b14_a16_Ref2A_nucleo- | GUAGUUGAGUGAGAGCGGUGAACGUUGAGUAGCGUGUGGAUG |
| capsid_targ_Bloop_ | CUAGCAAUUGCUAGCAUCGGUCGGGUCCGUUCACCGCUCUCA |
| antisense_A | (SEQ ID NO: 195) |
| rot_orange_broc_gen3_ | GGGAAUAAUACUGCGUCUUGGUUCACCGCUCUCACUCAACGC |
| b14_a17_Ref2A_nucleo- | AUGAGUUGAGUGAGAGCGGUGAACGUUGAGUAGCGUGUGGAC |
| capsid_targ_Bloop_ | CGUGGCUUGGGCCACGGUCGGUCGGGUCCGUUCACCGCUCUC |
| antisense_A | A (SEQ ID NO: 196) |
| rot_orange_broc_gen3_ | GGGUAAUACUGCGUCUUGGUUCACCGCUCUCACUCAACAUAC |
| b15_a15_Ref2A_nucleo- | CCUUGUUGAGUGAGAGCGGUGAACGUUGAGUAGCGUGUGGAG |
| capsid_targ_Bloop_ | AUUUAGUCUACUAAAUCUCGGUCGGGUCCGUUCACCGCUCUC |
| antisense_A | AC (SEQ ID NO: 197) |
| rot_orange_broc_gen3_ | GGGAUAAUACUGCGUCUUGGUUCACCGCUCUCACUCAACAUA |
| b15_a16_Ref2A_nucleo- | UCCCUGUUGAGUGAGAGCGGUGAACGUUGAGUAGCGUGUGGC |
| capsid_targ_Bloop_ | CAGACUGUGCUCAGUCUGGCGGUCGGGUCCGUUCACCGCUCU |
| antisense_A | CAC (SEQ ID NO: 198) |

TABLE 17
Sequences of the improved Corn RNA aptasensors tested in FIG. 22

| Name | Sequence |
|---|---|
| std_corn_gen3_b10_a15_ | GGGUACUGCCAGUUGAAUCUGAGGGUCCACCAAAGAACUAU |
| Ref2A_nucleocapsid_ | UUGGUGGACCCUCAGCGAGGAAGGAGGUCUGAGGAGGUCAC |
| targ_Floop_sense_A | UGCUGAGGGUCC (SEQ ID NO: 199) |
| std_corn_gen3_b12_a15_ | GGGUACUGCCAGUUGAAUCUGAGGGUCCACCAAACGGAUUG |
| Ref2A_nucleocapsid_ | GCGUUUGGUGGACCCUCAGCGAGGAAGGAGGUCUGAGGAGG |
| targ_Floop_sense_A | UCACUGCUGAGGGUCCAC (SEQ ID NO: 200) |
| std_corn_gen3_b13_a15_ | GGGUACUGCCAGUUGAAUCUGAGGGUCCACCAAACGUUCAC |
| Ref2A_nucleocapsid_ | AUACGUUUGGUGGACCCUCAGCGAGGAAGGAGGUCUGAGGA |
| targ_Floop_sense_A | GGUCACUGCUGAGGGUCCACC (SEQ ID NO: 201) |
| std_corn_gen3_b13_a16_ | GGGUUACUGCCAGUUGAAUCUGAGGGUCCACCAAACGUACA |
| Ref2A_nucleocapsid_ | UGAACGUUUGGUGGACCCUCAGCGAGGAAGGAGGUCUGAGG |
| targ_Floop_sense_A | AGGUCACUGCUGAGGGUCCACC (SEQ ID NO: 202) |
| std_corn_gen3_b14_a15_ | GGGAGUUGAAUCUGAGGGUCCACCAAACGUAAUGCGGGUUA |
| Ref2A_nucleocapsid_ | CCUCCCGCAUUACGUUUGGUGGACGAGGAAGGAGGUCUGAG |
| targ_Floop_sense_A | GAGGUCACUGUCCACCAAACGUAA (SEQ ID NO: 203) |
| std_corn_gen3_b14_a15_ | GGGUACUGCCAGUUGAAUCUGAGGGUCCACCAAACGUAUUC |
| Ref2A_nucleocapsid_ | AUUUACGUUUGGUGGACCCUCAGCGAGGAAGGAGGUCUGAG |
| targ_Floop_sense_B | GAGGUCACUGCUGAGGGUCCACCA (SEQ ID NO: 204) |
| std_corn_gen3_b14_a15_ | GGGAUAAUACUGCGUCUUGGUUCACCGCUCUCACUCAAGAU |
| Ref2A_nucleocapsid_ | AGAUUGAGUGAGAGCGGUGAACCCGAGGAAGGAGGUCUGAG |
| targ_Bloop_antisense_A | GAGGUCACUGGGUUCACCGCUCUC (SEQ ID NO: 205) |
| std_corn_gen3_b14_a16_ | GGGAAUAAUACUGCGUCUUGGUUCACCGCUCUCACUCAAUC |
| Ref2A_nucleocapsid_ | UUAUUUGAGUGAGAGCGGUGAACCCGAGGAAGGAGGUCUGA |
| targ_Bloop_antisense_A | GGAGGUCACUGGGUUCACCGCUCUC (SEQ ID NO: |
| | 206) |
| std_corn_gen3_b15_a15_ | GGGAUAAUACUGCGUCUUGGUUCACCGCUCUCACUCAACGG |
| Ref2A_nucleocapsid_ | ACUGGUUGAGUGAGAGCGGUGAACCCGAGGAAGGAGGUCUG |
| targ_Bloop_antisense_A | AGGAGGUCACUGGGUUCACCGCUCUCA (SEQ ID NO: |
| | 207) |
| std_corn_gen3_b15_a16_ | GGGAAUAAUACUGCGUCUUGGUUCACCGCUCUCACUCAACG |
| Ref2A_nucleocapsid_ | UGACAGUUGAGUGAGAGCGGUGAACCCGAGGAAGGAGGUCU |
| targ_Bloop_antisense_A | GAGGAGGUCACUGGGUUCACCGCUCUCA (SEQ ID NO: |
| | 208) |
| std_corn_gen3_b15_a19_ | GGGCAAGGUUUACCCAAUAAUACUGCGUCUUGGUUCACCGC |
| Ref2A_nucleocapsid_ | UCAGAAGCGAGCGGUGAACCAAGACGCAGCGAGGAAGGAGG |
| targ_Bloop_antisense_A | UCUGAGGAGGUCACUGCUGCGUCUUGGUUCA (SEQ ID |
| | NO: 209) |
| std_corn_gen3_b15_a19_ | GGGAAGGUUUACCCAAUAAUACUGCGUCUUGGUUCACCGCU |
| Ref2A_nucleocapsid_ | CUGGCACAAGAGCGGUGAACCAAGACGCACGAGGAAGGAGG |
| targ_Bloop_antisense_B | UCUGAGGAGGUCACUGUGCGUCUUGGUUCAC (SEQ ID |
| | NO: 210) |
| std_corn_gen3_b12_a15_ | GGGUUUUGGCAAUGUUGUUCCUUGAGGAAGUUGUAGAAUCA |
| Ref2B_nucleocapsid_ | GCUACAACUUCCUCAAGGACGAGGAAGGAGGUCUGAGGAGG |
| targ_Floop_sense_A | UCACUGUCCUUGAGGAAG (SEQ ID NO: 211) |
| std_corn_gen3_b12_a15_ | GGGUUUGGCAAUGUUGUUCCUUGAGGAAGUUGUAGCGGCAC |
| Ref2B_nucleocapsid_ | AGCUACAACUUCCUCAAGGCGAGGAAGGAGGUCUGAGGAGG |
| targ_Floop_sense_B | UCACUGCCUUGAGGAAGU (SEQ ID NO: 212) |
| std_corn_gen3_b12_a16_ | GGGUUUUGGCAAUGUUGUUCCUUGAGGAAGUUGUAGCUCUC |
| Ref2B_nucleocapsid_ | GUGCUACAACUUCCUCAAGGCGAGGAAGGAGGUCUGAGGAG |
| targ_Floop_sense_A | GUCACUGCCUUGAGGAAGU (SEQ ID NO: 213) |
| std_corn_gen3_b13_a15_ | GGGUUUUGGCAAUGUUGUUCCUUGAGGAAGUUGUAGCAGGG |
| Ref2B_nucleocapsid_ | UAGCUACAACUUCCUCAAGGACGAGGAAGGAGGUCUGAGGA |
| targ_Floop_sense_A | GGUCACUGUCCUUGAGGAAGU (SEQ ID NO: 214) |
| std_corn_gen3_b13_a16_ | GGGUUUUGGCAAUGUUGUUCCUUGAGGAAGUUGUAGCAGAU |
| Ref2B_nucleocapsid_ | CUAUGCUACAACUUCCUCAAGGCGAGGAAGGAGGUCUGAGG |
| targ_Floop_sense_A | AGGUCACUGCCUUGAGGAAGUU (SEQ ID NO: 215) |
| std_corn_gen3_b14_a15_ | GGGUUUUGGCAAUGUUGUUCCUUGAGGAAGUUGUAGCAAAG |
| Ref2B_nucleocapsid_ | UAGUGCUACAACUUCCUCAAGGACGAGGAAGGAGGUCUGAG |
| targ_Floop_sense_A | GAGGUCACUGUCCUUGAGGAAGUU (SEQ ID NO: 216) |
| std_corn_gen3_b13_a24_ | GGGUCAUCACGUAGUCGCAACAGUUCAAGAAAUUCAACUCC |
| Ref2B_nucleocapsid_ | AGGCAAUCAACUGCCUGGAGUUGAAUUUCUCGAGGAAGGAG |
| targ_Bloop_antisense_A | GUCUGAGGAGGUCACUGAGAAAUUCAACUC (SEQ ID |
| | NO: 217) |
| std_corn_gen3_b14_a15_ | GGGUAGUCGCAACAGUUCAAGAAAUUCAACUCCAGGCAGGC |
| Ref2B_nucleocapsid_ | GGAUGCCUGGAGUUGAAUUUCUUCGAGGAAGGAGGUCUGAG |
| targ_Bloop_antisense_A | GAGGUCACUGAAGAAAUUCAACUC (SEQ ID NO: 218) |
| std_corn_gen3_b14_a21_ | GGGUCACGUAGUCGCAACAGUUCAAGAAAUUCAACUCCAGG |
| Ref2B_nucleocapsid_ | CAGUGCACUCUGCCUGGAGUUGAAUUUCUCGAGGAAGGAGG |
| targ_Bloop_antisense_A | UCUGAGGAGGUCACUGAGAAAUUCAACUCC (SEQ ID |
| | NO: 219) |
| std_corn_gen3_b14_a24_ | GGGUCAUCACGUAGUCGCAACAGUUCAAGAAAUUCAACUCC |
| Ref2B_nucleocapsid_ | AGGCAGGUCACACUGCCUGGAGUUGAAUUUCUCGAGGAAGG |
| targ_Bloop_antisense_A | AGGUCUGAGGAGGUCACUGAGAAAUUCAACUCC (SEQ ID |
| | NO: 220) |
| std_corn_gen3_b15_a21_ | GGGUCACGUAGUCGCAACAGUUCAAGAAAUUCAACUCCAGG |
| Ref2B_nucleocapsid_ | CAGCUUAGUCGCUGCCUGGAGUUGAAUUUCUCGAGGAAGGA |
| targ_Bloop_antisense_A | GGUCUGAGGAGGUCACUGAGAAAUUCAACUCCA (SEQ ID |
| | NO: 221) |
| std_corn_gen3_b15_a24_ | GGGUCAUCACGUAGUCGCAACAGUUCAAGAAAUUCAACUCC |
| Ref2B_nucleocapsid_ | AGGCAGCACGACCGCUGCCUGGAGUUGAAUUUCUCGAGGAA |
| targ_Bloop_antisense_A | GGAGGUCUGAGGAGGUCACUGAGAAAUUCAACUCCA (SEQ |
| | ID NO: 222) |
| std_corn_gen3_b10_a15_ | GGGAAAAUAGAACCGCGGUCCUAUUCCAUUAUUCGAUAAAG |
| Ref16A_ctl_18S_rRNA_ | AAUAAUGGAAUAGGACGAGGAAGGAGGUCUGAGGAGGUCAC |
| targ_Floop_sense_A | UGUCCUAUUCCA (SEQ ID NO: 223) |
| std_corn_gen3_b11_a15_ | GGGAAAAUAGAACCGCGGUCCUAUUCCAUUAUUCCUCACCU |
| Ref16A_ctl_18S_rRNA_ | GGAAUAAUGGAAUAGGACGAGGAAGGAGGUCUGAGGAGGUC |
| targ_Floop_sense_A | ACUGUCCUAUUCCAU (SEQ ID NO: 224) |
| std_corn_gen3_b12_a15_ | GGGAAAAUAGAACCGCGGUCCUAUUCCAUUAUUCCUGCAGG |
| Ref16A_ctl_18S_rRNA_ | AAGGAAUAAUGGAAUAGGACGAGGAAGGAGGUCUGAGGAGG |
| targ_Floop_sense_A | UCACUGUCCUAUUCCAUU (SEQ ID NO: 225) |
| std_corn_gen3_b13_a15_ | GGGAAAAUAGAACCGCGGUCCUAUUCCAUUAUUCCUAGCAA |
| Ref16A_ctl_18S_rRNA_ | GAUAGGAAUAAUGGAAUAGGACGAGGAAGGAGGUCUGAGGA |
| targ_Floop_sense_A | GGUCACUGUCCUAUUCCAUUA (SEQ ID NO: 226) |
| std_corn_gen3_b14_a15_ | GGGAAAAUAGAACCGCGGUCCUAUUCCAUUAUUCCUAGUCA |
| Ref16A_ctl_18S_rRNA_ | CCUCUAGGAAUAAUGGAAUAGGACGAGGAAGGAGGUCUGAG |
| targ_Floop_sense_A | GAGGUCACUGUCCUAUUCCAUUAU (SEQ ID NO: 227) |
| std_corn_gen3_b15_a18_ | GGGCAAAAUAGAACCGCGGUCCUAUUCCAUUAUUCCUAGCU |
| Ref16A_ctl_18S_rRNA_ | GUAUGUUCAGCUAGGAAUAAUGGAAUAGCGAGGAAGGAGGU |
| targ_Floop_sense_A | CUGAGGAGGUCACUGCUAUUCCAUUAUUCC (SEQ ID |
| | NO: 228) |
| std_corn_gen3_b11_a17_ | GGGUUGGACCGGCGCAAGACGGACCAGAGCGAAAGCAGGAU |
| Ref16A_ctl_18S_rRNA_ | UAUGCUUUCGCUCUGGUCCCGAGGAAGGAGGUCUGAGGAGG |
| targ_Bloop_antisense_A | UCACUGGGACCAGAGCG (SEQ ID NO: 229) |
| std_corn_gen3_b13_a15_ | GGGAGAGGUGAAAUUCUUGGACCGGCGCAAGACGGACAAGU |
| Ref16A_ctl_18S_rRNA_ | AGGUCCGUCUUGCGCCGGUCCCGAGGAAGGAGGUCUGAGGA |
| targ_Bloop_antisense_A | GGUCACUGGGACCGGCGCAAG (SEQ ID NO: 230) |
| std_corn_gen3_b13_a17_ | GGGAGAGGUGAAAUUCUUGGACCGGCGCAAGACGGACCAUG |
| Ref16A_ctl_18S_rRNA_ | UUCUUGGUCCGUCUUGCGCCGGUCGAGGAAGGAGGUCUGAG |
| targ_Bloop_antisense_A | GAGGUCACUGACCGGCGCAAGAC (SEQ ID NO: 231) |
| std_corn_gen3_b14_a15_ | GGGAGAGGUGAAAUUCUUGGACCGGCGCAAGACGGACCAGC |
| Ref16A_ctl_18S_rRNA_ | UCCGGUCCGUCUUGCGCCGGUCCCGAGGAAGGAGGUCUGAG |
| targ_Bloop_antisense_A | GAGGUCACUGGGACCGGCGCAAGA (SEQ ID NO: 232) |
| std_corn_gen3_b14_a15_ | GGGAGGUGAAAUUCUUGGACCGGCGCAAGACGGACCAUCUA |
| Ref16A_ctl_18S_rRNA_ | UCUGGUCCGUCUUGCGCCGGUCCGAGGAAGGAGGUCUGAGG |
| targ_Bloop_antisense_B | AGGUCACUGGACCGGCGCAAGAC (SEQ ID NO: 233) |
| std_corn_gen3_b14_a16_ | GGGAGAGGUGAAAUUCUUGGACCGGCGCAAGACGGACCAGA |
| Ref16A_ctl_18S_rRNA_ | UGUAUGGUCCGUCUUGCGCCGGUCCGAGGAAGGAGGUCUGA |
| targ_Bloop_antisense_A | GGAGGUCACUGGACCGGCGCAAGAC (SEQ ID NO: |
| | 234) |
| std_corn_gen3_b11_a18_ | GGGAAGGUGUGGUGCCAGAUUUUCUCCAUGUCGUCCCAAUA |
| Ref17A_ctl_ACTB_mRNA_ | GGCUGGGACGACAUGGAGAACGAGGAAGGAGGUCUGAGGAG |
| targ_Floop_sense_A | GUCACUGUUCUCCAUGUC (SEQ ID NO: 235) |
| std_corn_gen3_b12_a15_ | GGGAAGGUGUGGUGCCAGAUUUUCUCCAUGUCGUCCAUACC |
| Ref17A_ctl_ACTB_mRNA_ | CGGACGACAUGGAGAAAAUCGAGGAAGGAGGUCUGAGGAGG |
| targ_Floop_sense_A | UCACUGAUUUUCUCCAUG (SEQ ID NO: 236) |
| std_corn_gen3_b12_a17_ | GGGAAGGUGUGGUGCCAGAUUUUCUCCAUGUCGUCCCACUG |
| Ref17A_ctl_ACTB_mRNA_ | GCCUGGGACGACAUGGAGAAACGAGGAAGGAGGUCUGAGGA |
| targ_Floop_sense_A | GGUCACUGUUUCUCCAUGUC (SEQ ID NO: 237) |
| std_corn_gen3_b13_a16_ | GGGAAGGUGUGGUGCCAGAUUUUCUCCAUGUCGUCCCAUUA |
| Ref17A_ctl_ACTB_mRNA_ | CAUUGGGACGACAUGGAGAAAACGAGGAAGGAGGUCUGAGG |
| targ_Floop_sense_A | AGGUCACUGUUUUCUCCAUGUC (SEQ ID NO: 238) |
| std_corn_gen3_b14_a16_ | GGGUGUGGUGCCAGAUUUUCUCCAUGUCGUCCCAGUUGCAG |
| Ref17A_ctl_ACTB_mRNA_ | UAAACUGGGACGACAUGGAGAACGAGGAAGGAGGUCUGAGG |
| targ_Floop_sense_A | AGGUCACUGUUCUCCAUGUCGUC (SEQ ID NO: 239) |
| std_corn_gen3_b15_a15_ | GGGAAGGUGUGGUGCCAGAUUUUCUCCAUGUCGUCCCAGCG |
| Ref17A_ctl_ACTB_mRNA_ | GUUACUGGGACGACAUGGAGAAAAUCGAGGAAGGAGGUCUG |
| targ__Floop_sense_A | AGGAGGUCACUGAUUUUCUCCAUGUCG (SEQ ID NO: |
| | 240) |
| std_corn_gen3_b10_a21_ | GGGAGAAGAUGACCCAGAUCAUGUUUGAGACCUUCAACACG |
| Ref17A_ctl_ACTB_mRNA_ | GAUAAGUGUUGAAGGUCUCAACGAGGAAGGAGGUCUGAGGA |
| targ_Bloop_antisense_A | GGUCACUGUUGAGACCUU (SEQ ID NO: 241) |
| std_corn_gen3_b12_a18_ | GGGAAGAUGACCCAGAUCAUGUUUGAGACCUUCAACACUUC |
| Ref17A_ctl_ACTB_mRNA_ | AUCGUGUUGAAGGUCUCAAACCGAGGAAGGAGGUCUGAGGA |
| targ_Bloop_antisense_A | GGUCACUGGUUUGAGACCUU (SEQ ID NO: 242) |
| std_corn_gen3_b15_a15_ | GGGAAGAUGACCCAGAUCAUGUUUGAGACCUUCAACACGUC |
| Ref17A_ctl_ACTB_mRNA_ | CGAGUGUUGAAGGUCUCAAACAUGCGAGGAAGGAGGUCUGA |
| targ_Bloop_antisense_A | GGAGGUCACUGCAUGUUUGAGACCUU (SEQ ID NO: |
| | 243) |
| std_corn_gen3_b15_a16_ | GGGAGAAGAUGACCCAGAUCAUGUUUGAGACCUUCAACACG |
| Ref17A_ctl_ACTB_mRNA_ | ACUGAGUGUUGAAGGUCUCAAACAUGCGAGGAAGGAGGUCU |
| targ_Bloop_antisense_A | GAGGAGGUCACUGCAUGUUUGAGACCUU (SEQ ID NO: |
| | 244) |
| std_corn_gen3_b15_a16_ | GGGCGAGAAGAUGACCCAGAUCAUGUUUGAGACCUUCAACA |
| Ref17A_ctl_ACTB_mRNA_ | GUGAAGUUGAAGGUCUCAAACAUGAUCGAGGAAGGAGGUCU |
| targ_Bloop_antisense_B | GAGGAGGUCACUGAUCAUGUUUGAGACC (SEQ ID NO: |
| | 245) |
| std_corn_gen3_b15_a17_ | GGGAGAAGAUGACCCAGAUCAUGUUUGAGACCUUCAACACG |
| Ref17A_ctl_ACTB_mRNA_ | AGGAAGUGUUGAAGGUCUCAAACAUGCGAGGAAGGAGGUCU |
| targ_Bloop_antisense_A | GAGGAGGUCACUGCAUGUUUGAGACCUU (SEQ ID NO: |
| | 246) |

TABLE 18
Sequences of the RNA aptasensors targeting various loops
within RT-LAMP amplicons tested in FIG. 25

| Name | Sequence |
|---|---|
| broc_gen2_b11_a17_ | GGGAGUUGAAUCUGAGGGUCCACCAAACGUAAUGCGGAAUCGA |
| Ref2A_nucleocapsid_ | CCGCAUUACGUUUGGUGUCGAGUAGAGUGUGGGCUCAGAUUCG |
| targ_Floop_sense_B | UCUGAGACGGUCGGGUCCACCAAACGUA (SEQ ID NO: |
| | 247) |
| broc_gen2_b12_a22_ | GGGAAUAAUACUGCGUCUUGGUUCACCGCUCUCACUCAACAUG |
| Ref2A_nucleocapsid_ | CAAUUACAUGUUGAGUGAGAGCGGUCGAGUAGAGUGUGGGCUC |
| targ_Bloop_anti- | AGAUUCGUCUGAGACGGUCGGGUCCCGCUCUCACUC (SEQ |
| sense_A | ID NO: 248) |

TABLE 19
Sequence of the Broccoli RNA aptasensor for detection of
SARS-COV-2 in RT-LAMP amplicons tested in FIG. 26

| Name | Sequence |
|---|---|
| broc_rot_b10_ | GGGAUAACCCUGUCCUACCAUUUAAUGAUGGUGUUUAGAAACGGA |
| Ref2C_spike_targ_ | UAAUCACAAUCAUUAAAUGGUCGAGUAGAGUGUGGGCUCAGAUUC |
| Bloop_antisense | GUCUGAGACGGUCGGGUCCCAUUUAAUG (SEQ ID NO: 121) |

TABLE 20
Sequences of the Broccoli RNA aptasensors for detection of
control ACTB mRNAs in RT-LAMP amplicons tested in FIG. 27

| Name | Sequence |
|---|---|
| broc_gen2_b12_a15_ | GGGUGCCAGAUUUUCUCCAUGUCGUCCCAGUUGGGGACAGC |
| Ref17A_ctl_ACTB_ | CAACUGGGACGACAUGGUCGAGUAGAGUGUGGGCUCAGAUU |
| mRNA_targ_Floop_ | CGUCUGAGACGGUCGGGUCCCAUGUCGUCCC (SEQ ID |
| sense_A | NO: 249) |
| broc_gen2_b11_a15_ | GGGAGAUCAUGUUUGAGACCUUCAACACCCCAGCCAGCUAA |
| Ref17A_ctl_ACTB_ | GGCUGGGGUGUUGAAGGUCGAGUAGAGUGUGGGCUCAGAUU |
| mRNA_targ_Bloop_ | CGUCUGAGACGGUCGGGUCCCUUCAACACC (SEQ ID |
| antisense_A | NO: 250) |

TABLE 21
Sequence of the Red Broccoli RNA aptasensor for detection of
SARS-COV-2 in RT-LAMP amplicons tested in FIG. 28

| Name | Sequence |
|---|---|
| std_red_broc_gen3_ | GGGUCAUCACGUAGUCGCAACAGUUCAAGAAAUUCAACUCCAG |
| b15_a28_Ref2B_ | GCAGCAGUAUGAAGUUACUGCUGCCUGGAGUUGAAUCGGUCGG |
| nucleocapsid_targ_ | GUCCGGAAUAACUCGGGUUAUUCCGUUGAGUAGUGUGUGGAUU |
| Bloop_antisense_A | CAACUCCAGGCA (SEQ ID NO: 166) |

TABLE 22
Sequences of the Red Broccoli RNA aptasensors for detection of
control ACTB mRNAs in RT-LAMP amplicons tested in FIG. 29

| Name | Sequence |
|---|---|
| rot_red_broc_gen3_b11_ | GGGUGUGGUGCCAGAUUUUCUCCAUGUCGUCCCAGAUGGCC |
| a17_Ref17A_ctl_ACTB_ | CUGGGACGACAUGGAGAGUUGAGUAGUGUGUGGAGGUCCUU |
| mRNA_targ_Floop_sense_ | GUACAAGGACCUCGGUCGGGUCCUCUCCAUGUCG (SEQ |
| A | ID NO: 251) |
| std_red_broc_gen3_b15_ | GGGCGAGAAGAUGACCCAGAUCAUGUUUGAGACCUUCAAUU |
| a16_Ref17A_ctl_ACTB_ | ACCUUUGAAGGUCUCAAACAUGAUCCGGUCGGGUCCGACUU |
| mRNA_targ_Bloop_anti- | UCCUGUGGGAAAGUCGUUGAGUAGUGUGUGGGAUCAUGUUU |
| sense_A | GAGAC (SEQ ID NO: 252) |

TABLE 23
Sequences of the Orange Broccoli RNA aptasensors for detection
of RT-LAMP amplicons tested in FIG. 30

| Name | Sequence |
|---|---|
| std_orange_broc_gen3_ | GGGCAUCACGUAGUCGCAACAGUUCAAGAAAUUCAACUCCAG |
| b13_a31_Ref2B_nucleo- | GCAGCAGUAGGUUUAGUCCUACUGCUGCCUGGAGUUCGGUCG |
| capsid_targ_Bloop_ | GGUCCGGUGUAUGGUGACAUACACCGUUGAGUAGCGUGUGGA |
| antisense_A | ACUCCAGGCAGC (SEQ ID NO: 253) |
| std_orange_broc_gen3_ | GGGUGUUGUUCCUUGAGGAAGUUGUAGCACGAUUGCAUGAAA |
| b13_a15_Ref2B_nucleo- | UUGCAAUCGUGCUACAACUUCGGUCGGGUCCGCCUUAUCAGC |
| capsid_targ_Floop_ | CGAUAAGGCGUUGAGUAGCGUGUGGAAGUUGUAGCACG |
| sense_A | (SEQ ID NO: 254) |
| rot_orange_broc_gen3_ | GGGUGUGGUGCCAGAUUUUCUCCAUGUCGUCCCAGUUUGUCU |
| b12_a17_Ref17A_ctl_ | UAACUGGGACGACAUGGAGGUUGAGUAGCGUGUGGGCUAUUU |
| ACTB_mRNA_targ_Floop_ | CAGCUGAAAUAGCCGGUCGGGUCCCUCCAUGUCGUC (SEQ |
| sense_A | ID NO: 255) |
| std_orange_broc_gen3_ | GGGAGAAGAUGACCCAGAUCAUGUUUGAGACCUUCAAUUUGC |
| b14_a15_Ref17A_ctl_ | UUUGAAGGUCUCAAACAUGAUCGGUCGGGUCCGGCAUGCCUC |
| ACTB_mRNA_targ_Bloop_ | CAGGCAUGCCGUUGAGUAGCGUGUGGAUCAUGUUUGAGAC |
| antisense_A | (SEQ ID NO: 256) |

TABLE 24
Sequences of the Corn RNA aptasensors for detection of
control ACTB mRNAs in RT-LAMP amplicons tested in FIG. 31

| Name | Sequence |
|---|---|
| Corn_sta_arb_b11_ | GGGCGAGAAGAUGACCCAGAUCAUGUUUGAGACCUUCACAU |
| Ref17A_ctl_ACTB_ | AACACUGACGGUAUCAAACAUGAUCGAGGAAGGAGGUCUGA |
| mRNA_targ_Bloop_ | GGAGGUCACUGAUCAUGUUUGACA (SEQ ID NO: 143) |
| antisense | |

TABLE 25
Sequences of the Broccoli and Corn RNA aptasensors tested
in a two-channel RT-LAMP/aptasensor assay in FIG. 33

| Name | Sequence |
|---|---|
| Corn_sta_arb_b11_ | GGGCGAGAAGAUGACCCAGAUCAUGUUUGAGACCUUCACAUA |
| Ref17A_ctl_ACTB_ | ACACUGACGGUAUCAAACAUGAUCGAGGAAGGAGGUCUGAGG |
| mRNA_targ_Bloop_ | AGGUCACUGAUCAUGUUUGACA (SEQ ID NO: 143) |
| antisense | |
| broc_rot_b10_Ref2C_ | GGGAUAACCCUGUCCUACCAUUUAAUGAUGGUGUUUAGAAAC |
| spike_targ_Bloop_ | GGAUAAUCACAAUCAUUAAAUGGUCGAGUAGAGUGUGGGCUC |
| antisense | AGAUUCGUCUGAGACGGUCGGGUCCCAUUUAAUG (SEQ ID |
| | NO: 121) |

TABLE 26
Sequences of the Broccoli and Corn RNA aptasensors tested
in a one-pot RT-LAMP/aptasensor assay in FIG. 35

| Name | Sequence |
|---|---|
| Corn_sta_arb_b11_ | GGGCGAGAAGAUGACCCAGAUCAUGUUUGAGACCUUCACAUA |
| Ref17A_ctl_ACTB_ | ACACUGACGGUAUCAAACAUGAUCGAGGAAGGAGGUCUGAGG |
| mRNA_targ_Bloop_ | AGGUCACUGAUCAUGUUUGACA (SEQ ID NO: 143) |
| antisense | |
| broc_rot_b10_Ref2C_ | GGGAUAACCCUGUCCUACCAUUUAAUGAUGGUGUUUAGAAAC |
| spike_targ_Bloop_ | GGAUAAUCACAAUCAUUAAAUGGUCGAGUAGAGUGUGGGCUC |
| antisense | AGAUUCGUCUGAGACGGUCGGGUCCCAUUUAAUG (SEQ ID |
| | NO: 121) |

TABLE 27
Sequences of the Broccoli and Corn RNA aptasensors tested in
a two-channel, one-pot RT-LAMP/aptasensor assay in FIG. 36

| Name | Sequence |
|---|---|
| broc_gen2_b11_a17_ | GGGAGUUGAAUCUGAGGGUCCACCAAACGUAAUGCGGAAUCG |
| Ref2A_nucleocapsid_ | ACCGCAUUACGUUUGGUGUCGAGUAGAGUGUGGGCUCAGAUU |
| targ_Floop_sense_B | CGUCUGAGACGGUCGGGUCCACCAAACGUA (SEQ ID NO: |
| | 247) |
| broc_gen2_b12_a22_ | GGGAAUAAUACUGCGUCUUGGUUCACCGCUCUCACUCAACAU |
| Ref2A_nucleocapsid_ | GCAAUUACAUGUUGAGUGAGAGCGGUCGAGUAGAGUGUGGGC |
| targ_Bloop_anti- | UCAGAUUCGUCUGAGACGGUCGGGUCCCGCUCUCACUC |
| sense_A | (SEQ ID NO: 248) |
| Corn_sta_arb_b11_ | GGGCGAGAAGAUGACCCAGAUCAUGUUUGAGACCUUCACAUA |
| Ref17A_ctl_ACTB_ | ACACUGACGGUAUCAAACAUGAUCGAGGAAGGAGGUCUGAGG |
| mRNA_targ_Bloop_ | AGGUCACUGAUCAUGUUUGACA (SEQ ID NO: 143) |
| antisense | |

We claim:

1. An aptasensor comprising:
a) a target-binding sequence that is complementary to a SARS-CoV-2 target nucleic acid or to the complement thereof; and
b) an aptamer;
wherein, in the absence of the SARS-CoV-2 target nucleic acid, the aptasensor forms a stem-loop structure in which a first portion of the target-binding sequence forms a single-stranded toehold and a second portion of the target-binding sequence base-pairs with a portion of the aptamer to form a stem, such that the aptamer cannot fold into its active form; and
wherein binding of the target-binding sequence to the SARS-CoV-2 target nucleic acid disrupts the stem-loop structure, allowing the aptamer to fold into its active form and bind to its cognate ligand.

2. The aptasensor of claim 1, wherein binding of the aptamer to its cognate ligand produces a detectable signal.

3. The aptasensor of claim 2, wherein the detectable signal is a fluorescence signal.

4. The aptasensor of claim 3, wherein the aptamer is selected from the group consisting of:
Broccoli, Corn, Spinach, Spinach2, Carrot, Radish, Red Broccoli, Orange Broccoli, a G-quadruplex-containing aptamer, and a malachite green binding aptamer.

5. The aptasensor of any one of the preceding claims, wherein the SARS-CoV-2 target nucleic acid is a portion of a SARS-CoV-2 gene selected from the group consisting of: Orflb, RdRp, spike, E, and N.

6. The aptasensor of claim 5, wherein the aptasensor comprises a sequence selected from SEQ ID NOs:1-118, 121-136, and 151-248.

7. The aptasensor of any one of the preceding claims, wherein the aptasensor consists of RNA.

8. The aptasensor of any one of the preceding claims, wherein a stem of the stem-loop structure is 10-25 nucleotides in length.

9. The aptasenor of claim 8, wherein the stem is at least 18 nucleotides in length and comprises at least one non-base paired nucleotide.

10. The aptasensor of claim 8, wherein the stem is 12-21 nucleotides in length and each base in the stem is base-paired.

11. The aptasensor of any one of the preceding claims, wherein a loop of the stem-loop structure is 6-10 nucleotides in length, optionally the loop is 8 nucleotides in length.

12. The aptasensor of any one of the preceding claims, wherein the aptamer comprises an inner clamp that forms a stem-loop structure when the aptamer is in its active form, wherein the stem of the stem-loop structure is 6-14 nucleotides in length and the loop of the stem-loop structure is 4-10 nucleotides in length, optionally the stem is 8 nucleotides in length and the loop is 4 nucleotides in length.

13. A method of detecting SARS-CoV-2 in a sample, the method comprising:
a) amplifying the SARS-CoV-2 target nucleic acid in the sample;
b) contacting the amplified nucleic acid with the aptasensor of any one of claims 1-12 and the cognate ligand of its aptamer; and
c) detecting any signal produced by the aptamer binding to its cognate ligand, wherein detection of the signal indicates that SARS-CoV-2 is present in the sample.

14. The method of claim 13, wherein the SARS-CoV-2 target nucleic acid is detectable at a concentration as low as 0.13 aM.

15. The method of claim 13 or 14, wherein the signal, if present, is detectable in less than 1 hour.

16. The method of claim 15, wherein the signal, if present, is detectable in less than 30 minutes.

17. The method of any one of claims 13-16, wherein the amplification step is performed using an isothermal amplification method.

18. The method of claim 17, wherein the isothermal amplification method is selected from the group consisting of: nucleic acid sequence-based amplification (NASBA), reverse transcription recombinase polymerase amplification (RT-RPA), and reverse transcription loop-mediated isothermal amplification (RT-LAMP).

19. The method of any one of claims 13-18 further comprising amplifying a control nucleic acid in the sample and detecting the amplified control nucleic acid.

20. The method of claim 19, wherein the control nucleic acid is selected from the group consisting of: human RNase P mRNA, beta actin (ACTB) mRNA, and 18S rRNA.

21. The method of any one of claims 13-20 further comprising heat inactivating any SARS-CoV-2 virions in the sample prior to step (a).

22. A kit comprising an aptasensor of any one of claims 1-12.

23. The kit of claim 22, further comprising primers that can be used to specifically amplify the SARS-CoV-2 target nucleic acid.

24. An aptasensor comprising:
a) a target-binding sequence that is complementary to a target nucleic acid or to the complement thereof; and
b) an aptamer;
wherein, in the absence of the target nucleic acid, the aptasensor forms a stem-loop structure in which a first portion of the target-binding sequence forms a single-stranded toehold and a second portion of the target-binding sequence base-pairs with a portion of the aptamer to form a stem, such that the aptamer cannot fold into its active form; and
wherein binding of the target-binding sequence to the target nucleic acid disrupts the stem-loop structure, allowing the aptamer to fold into its active form and bind to its cognate ligand,
wherein the aptamer comprises an inner clamp that forms a stem-loop structure when the aptamer is in its active form, wherein the stem of the stem-loop structure is 6-14 nucleotides in length and the loop of the stem-loop structure is 4-10 nucleotides in length, optionally the stem is 8 nucleotides in length and the loop is 4 nucleotides in length.
